(19) United States
(12) Patent Application Publication
(10) Pub. No.: US 2018/0037568 A1
(43) Pub. Date: Feb. 8, 2018

(54) MODULATORS OF METHYL MODIFYING ENZYMES, COMPOSITIONS AND USES THEREOF

(71) Applicant: Constellation Pharmaceuticals, Inc., Cambridge, MA (US)

(72) Inventors: Brian K. Albrecht, Cambridge, MA (US); Victor S. Gehling, Somerville, MA (US); Jean-Christophe Harmange, Andover, MA (US); Rishi G. Vaswani, Lexington, MA (US)

(21) Appl. No.: 15/549,265

(22) PCT Filed: Feb. 4, 2016
(86) PCT No.: PCT/US2016/016546

§ 371 Date: Aug. 7, 2017

Related U.S. Application Data
(60) Provisional application No. 62/115,936, filed on Feb. 13, 2015.

(51) Int. Cl. C07D 401/14; C07D 405/14

(52) U.S. Cl. CPC C07D 401/14; C07D 405/14

(57) ABSTRACT

Provided are novel compounds of Formula (I): and pharmaceutically acceptable salts thereof, which are useful for treating a variety of diseases, disorders or conditions, associated with methyl modifying enzymes. Also provided are pharmaceutical compositions comprising the novel compounds of Formula (I), pharmaceutically acceptable salts thereof, and methods for their use in treating one or more diseases, disorders or conditions, associated with methyl modifying enzymes.

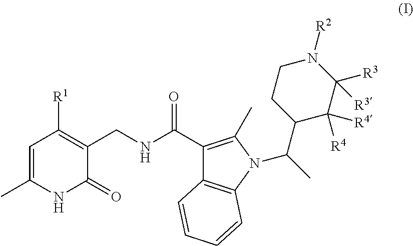

### CROSS REFERENCE TO RELATED APPLICATIONS

This application claims priority U.S. Provisional Application No. 62/115,936, filed Feb. 13, 2015, the entire contents of which are incorporated herein by reference.

### BACKGROUND OF THE INVENTION

Eukaryotic chromatin is composed of macromolecular complexes called nucleosomes. A nucleosome has 147 base pairs of DNA wrapped around a protein octamer having two subunits of each of histone protein H2A, H2B, H3, and H4. Histone proteins are subject to post-translational modifications which in turn affect chromatin structure and gene expression. One type of post-translational modification found on histones is methylation of lysine and arginine residues. Histone methylation plays a critical role in the regulation of gene expression in eukaryotes. Methylation affects chromatin structure and has been linked to both activation and repression of transcription (Zhang and Reinberg, Genes Dev. 15:2343-2360, 2001). Enzymes that catalyze attachment and removal of methyl groups from histones are implicated in gene silencing, embryonic development, cell proliferation, and other processes.

One class of histone methylases is characterized by the presence of a SET domain, comprising about 130 amino acids. EZH2 is an example of a human SET-domain containing methylase. EZH2 associates with EED (Embryonic Ectoderm Development) and SUZ12 (suppressor of zeste 12 homolog) to form a complex known as PRC2 (Polycomb Group Repressive Complex 2) having the ability to tri-methylate histone H3 at lysine 27 (Cao and Zhang, Mol. Cell 15:57-67, 2004). PRC2 complexes can also include RBAP46 and RBAP48 subunits. Another example is the related methylase EZH1.

The oncogenic activities of EZH2 have been shown by a number of studies. In cell line experiments, over-expression of EZH2 induces cell invasion, growth in soft agar, and motility while knockdown of EZH2 inhibits cell proliferation and cell invasion (Kleer et al., 2003, Proc. Nat. Acad. Sci. USA 100:11606-11611; Varambally et al., (2002), “The polycomb group protein EZH2 is involved in progression of prostate cancer,” Nature 419, 624-629). It has been shown that EZH2 represses the expression of several tumor suppressors, including E-cadherin, DAB2IP and RUNX3 among others. In xenograft models, EZH2 knockdown inhibits tumor growth and metastasis. Recently, it has been shown that down modulation of EZH2 in murine models blocks prostate cancer metastasis (Min et al., “An oncogene-tumor suppressor cascade drives metastatic prostate cancer by coordinately activating Ras and nuclear factor-kappaB,” Nat Med. 2010 March; 16(3):286-94). EZH2 overexpression is associated with aggressiveness of certain cancers such as breast cancer (Kleer et al., Proc. Nat. Acad. Sci. USA 100:11606-11611, 2003). Recent studies also suggest that prostate cancer specific oncogenic fusion gene TMPRSS2-ERG induces repressive epigenetic programs via direct activation of EZH2 (Yu et al., “An Integrated Network of Androgen Receptor, Polycomb, and TMPRSS2-ERG Gene Fusions in Prostate Cancer Progression,” Cancer Cell. 2010 May 18; 17(5):443-454).

### SUMMARY OF THE INVENTION

The present disclosure encompasses the recognition that methyl modifying enzymes, in particular EZH2 and mutant forms thereof, are an attractive target for modulation, given their role in the regulation of diverse biological processes. It has now been found that compounds described herein, and pharmaceutically acceptable compositions thereof, are effective as agents that modulate the activity of EZH2 (See e.g., Table 2). Such compounds include those of structural formula I:

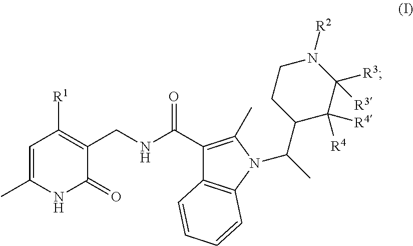

or a pharmaceutically acceptable salt thereof, wherein each of R¹, R², R³, R³′, R⁴, and R^{4′} are defined and described herein.

Compounds described herein, and pharmaceutically acceptable compositions thereof, are useful for treating a variety of diseases, disorders or conditions, associated with a methyl modifying enzyme. Such diseases, disorders, or conditions include those described herein.

Compounds described herein are also useful for the study of methyl modifying enzymes in biological and pathological phenomena; the study of intracellular signal transduction pathways mediated by methyl modifying enzymes and the comparative evaluation of new methyl modifying enzyme modulators.

### DETAILED DESCRIPTION OF CERTAIN EMBODIMENTS

### 1. General Description of Compounds of the Invention

In certain embodiments, the present disclosure provides a compound of Formula I:

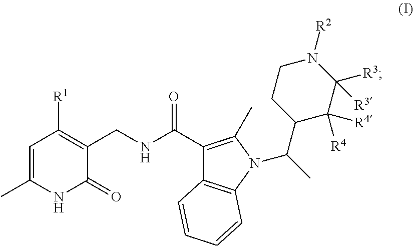

or a pharmaceutically acceptable salt thereof, wherein

R¹ is (C₁-C₄)alkyl, (C₁-C₃)alkoxy, or OCHF₂;

R² is hydrogen, methyl, ethyl, thiazolyl, —SO₂(C₁-C₃)alkyl, —COCH₃, —COOCH₃, —C(CH₃)₂NH(C₁-C₃)alkyl, —C(CH₃)₂COOH, —CH₂R⁵, —C(O)OR⁶, —C(O)R⁷, —CHR⁸R⁹, —CH₂(CO)R¹⁰, isoxazolyl substituted with (C₁-C₃)alkyl, or oxetanyl optionally substituted with hydroxycarbonyl(C₁-C₃)alkyl or hydroxy(C₁-C₃)alkyl;

R³, R³′, R⁴ and R^{4′} are each independently hydrogen, CF₃, CH₃, CH₂CH₃, CH₂F, CHF₂, CH₂CF₃, ═O, CH₂OH, or phenyl optionally substituted with (C₁-C₃)alkyl, halo(C₁-C₃)alkyl, halo, cyano, or COOH, provided that at least one of R³, R³′, R⁴ and R^{4′} is not hydrogen; or

R³ and R⁴ or R^{3′} and R^{4′} are taken together form a phenyl or cyclohexyl ring;

R⁵ is cyclopropyl, CHF₂, —C(CH₃)₂COOCH₃, —C(CH₃)₂COOH, —CF₂CH₂pyrrolidinyl, -phenyl(CO)phenyl, —CH₂OCF₃, —C(CH₃)₂CH₂NH₂, CF₃, —CH₂NHCH₂CF₃, pyridinyl substituted with (C₂-C₄)alkyl, or isoxazolyl substituted with one or more (C₁-C₃)alkyl;

R⁶ is di(C₁-C₃)alkylamino(C₁-C₃)alkyl, pyrrolidinyl substituted with (C₁-C₃)alkyl, cyclopropyl substituted with (C₁-C₃)alkyl, azetidinyl substituted with (C₁-C₃)alkyl, or piperidinyl substituted with (C₁-C₃)alkyl;

R⁷ is —CH₂-cyclopentyl, —CH₂-cyclopropyl, —CH₂-tetrahydropyranyl, —CH₂NHCH₂CF₃, pyridazinyl, —CH₂CF₃, piperidinyl substituted with (C₁-C₃)alkoxy, —CH₂-azetidinyl substituted with one or more fluoro, pyrazinyl substituted with (C₁-C₃)alkyl, imidazolyl substituted with (C₁-C₃)alkyl, pyridinyl substituted with (C₁-C₃)alkyl, or pyrrolidinyl substituted with (C₁-C₃)alkoxy or hydroxyl;

R⁸ is CF₃, CH₃, or —NOCH₃;

R⁹ is pyridinyl optionally substituted with one or two (C₁-C₃)alkyl, phenyl substituted with cyano or one or more halo, or pyrazolyl substituted with one or two groups selected from (C₁-C₃)alkyl and halo; and

R¹⁰ is NH(C₁-C₃)alkyl, (C₂-C₄)alkyl, cyclopenyl, cyclobutyl optionally substituted with CF₃, or cyclopropyl optionally substituted with (C₁-C₃)alkyl, halo, or CF₃.

### 2. Compounds and Definitions

The terms “halo” and “halogen” as used herein refer to an atom selected from fluorine (fluoro, —F), chlorine (chloro, —Cl), bromine (bromo, —Br), and iodine (iodo, —I).

The term “alkyl,” as used herein, refers to a monovalent saturated, straight- or branched-chain hydrocarbon radical, having unless otherwise specified, 1-10 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The term “alkoxy,” as used herein, refers to an alkyl group which is attached to another moiety via an oxygen atom (—O(alkyl)). Non-limiting examples include e.g., methoxy, ethoxy, propoxy, and butoxy.

The term “haloalkyl” includes mono, poly, and perhaloalkyl groups where the halogens are independently selected from fluorine, chlorine, bromine, an iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

Certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms.

“Racemate” or “racemic mixture” means a compound of equimolar quantities of two enantiomers, wherein such mixtures exhibit no optical activity, i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule.

The compounds of the invention may be prepared as individual enantiomers by either enantio-specific synthesis or resolved from an enantiomerically enriched mixture. Conventional resolution techniques include forming the salt of a free base of each isomer of an enantiomeric pair using an optically active acid (followed by fractional crystallization and regeneration of the free base), forming the salt of the acid form of each enantiomer of an enantiomeric pair using an optically active amine (followed by fractional crystallization and regeneration of the free acid), forming an ester or amide of each of the enantiomers of an enantiomeric pair using an optically pure acid, amine or alcohol (followed by chromatographic separation and removal of the chiral auxiliary), or resolving an enantiomeric mixture of either a starting material or a final product using various well known chromatographic methods.

Unless otherwise specified, when the stereochemistry of a disclosed compound is named or depicted by structure, the named or depicted stereoisomer is at least 60%, 70%, 80%, 90%, 99% or 99.9% by weight pure relative to all of the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

Unless otherwise specified, when a disclosed compound is named or depicted by structure without indicating the stereochemistry, and the compound has at least one chiral center, it is to be understood that the name or structure encompasses one enantiomer of compound free from the corresponding optical isomer, a racemic mixture of the compound and mixtures enriched in one enantiomer relative to its corresponding optical isomer.

Unless otherwise specified, when a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

As used herein, the term “inhibitor” is defined as a compound that binds to and/or inhibits a target S-adenosylmethionine (SAM) utilizing enzyme with measurable affinity. In certain embodiments, an inhibitor has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in activity of at least one SAM utilizing enzyme between a sample comprising a provided compound, or composition thereof, and at least one SAM dependent enzyme, and an equivalent sample comprising at least one SAM dependent enzyme, in the absence of said compound, or composition thereof.

### 3. Description of Exemplary Compounds

In a first embodiment, the present disclosure provides a compound of Formula I:

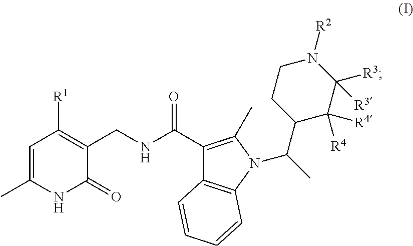

or a pharmaceutically acceptable salt thereof, wherein the variables are as described herein.

In a second embodiment, the compound of Formula I is of Formula Ia:

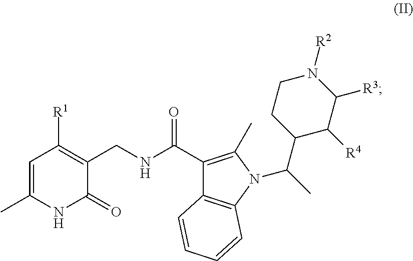

or a pharmaceutically acceptable salt thereof, wherein the variables in Formula II are as described in Formula I.

In a third embodiment, the compound of Formula I is of Formula III:

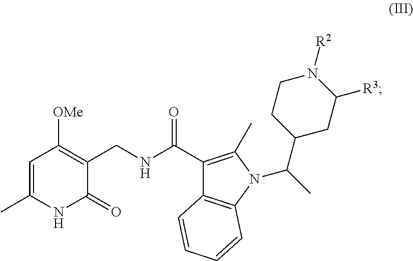

or a pharmaceutically acceptable salt thereof, wherein the variables in Formula III are as described in Formula I.

In a fourth embodiment, R³ in Formulas I, II, and III is CF₃, CH₂CH₃, CH₃, or halophenyl, wherein the remaining variables are as described in Formula I.

In a fifth embodiment, R² in Formulas I, II, and III is hydrogen, methyl, ethyl, thiazolyl, —C(CH₃)₂NH(C₁-C₃)alkyl, —C(CH₃)₂COOH, —CH₂R⁵, —C(O)OR⁶, —C(O)R⁷, —CHR⁸R⁹, —CH₂(CO)R¹⁰, or isoxazolyl substituted with (C₁-C₃)alkyl, wherein the remaining variables are as described in Formula I or the third embodiment. Alternatively, R² is hydrogen, methyl, ethyl, —CH₂R⁵, —C(O)OR⁶, —C(O)R⁷, —CHR⁸R⁹, or —CH₂(CO)R¹⁰, wherein the remaining variables are as described in Formula I or the fourth embodiment. In another alternative, R² is hydrogen, methyl, ethyl, —CH₂R⁵, or —C(O)R⁷, wherein the remaining variables are as described in Formula I or the fourth embodiment.

In a sixth embodiment, R⁵ in Formulas I, II, and III is cyclopropyl, CHF₂, -phenyl(CO)phenyl, —CH₂OCF₃, CF₃, pyridinyl substituted with (C₂-C₄)alkyl, or isoxazolyl substituted with one or more (C₁-C₃)alkyl, wherein the remaining variables are as described in Formula I or the fourth or fifth embodiment. Alternatively, R⁵ is CHF₂, CF₃, or pyridinyl substituted with (C₂-C₄)alkyl, wherein the remaining variables are as described in Formula I or the fourth or fifth embodiment. In another alternative, R⁵ is CHF₂ or CF₃, wherein the remaining variables are as described in Formula I or the fourth or fifth embodiment.

In a seventh embodiment, R⁶ in Formulas I, II, and III is cyclopropyl substituted with (C₁-C₃)alkyl or piperidinyl substituted with (C₁-C₃)alkyl, wherein the remaining variables are as described in Formula I or the fourth, fifth, or sixth embodiment.

In an eighth embodiment, R⁷ in Formulas I, II, and III is —CH₂-cyclopentyl, —CH₂-cyclopropyl, —CH₂NHCH₂CF₃, —CH₂CF₃, pyrazinyl substituted with (C₁-C₃)alkyl, imidazolyl substituted with (C₁-C₃)alkyl, or pyrrolidinyl substituted with (C₁-C₃)alkoxy, wherein the remaining variables are as described in Formula I or the fourth, fifth, sixth, or seventh embodiment.

In a ninth embodiment, R⁸ in Formulas I, II, and III is CF₃ or CH₃, wherein the remaining variables are as described in Formula I or the fourth, fifth, sixth, seventh, or eighth embodiment.

In a tenth embodiment, R⁹ in Formulas I, II, and III is pyridinyl optionally substituted with one or two (C₁-C₃)alkyl; or phenyl substituted with cyano or one or more halo, wherein the remaining variables are as described in Formula I or the fourth, fifth, sixth, seventh, eighth, or ninth embodiment.

In an eleventh embodiment, R¹⁰ in Formulas I, II, and III is NH(C₁-C₃)alkyl, (C₂-C₄)alkyl, cyclopenyl, cyclobutyl optionally substituted with CF₃, or cyclopropyl optionally substituted with (C₁-C₃)alkyl, halo, or CF₃, wherein the remaining variables are as described in Formula I or the fourth, fifth, sixth, seventh, eighth, ninth, or tenth embodiment.

In a twelfth embodiment, the compound of Formula I is of Formula IV:

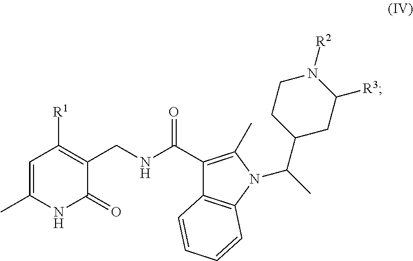

or a pharmaceutically acceptable salt thereof, wherein R¹ is (C₁-C₄)alkyl or (C₁-C₃)alkoxy; R² is hydrogen, methyl, ethyl, oxetanyl, —CH₂cyclopropyl, —CH₂CHF₂, CH₂CF₃, —COCH₃, or —SO₂Et; and R³ is methyl, ethyl, CF₃, ═O, —CH₂CH₂F, —CH₂OH, —CHF₂, —CH₂CF₃, halophenyl, or trifluoromethylphenyl.

In a thirteenth embodiment, R¹ in Formula IV is methyl or methoxy, wherein the remaining variables are as described in Formula IV.

In a fourteenth embodiment, R² in Formula IV is hydrogen, methyl, or ethyl; and R³ is CF₃ or halophenyl, wherein the remaining variables are as described in Formula IV or the thirteenth embodiment. Alternatively, R² is hydrogen or methyl; and R³ is CF₃ or fluorophenyl, wherein the remaining variables are as described in Formula IV or the thirteenth embodiment.

In a fifteenth embodiment, R³ in Formula IV is CF₃, wherein the remaining variables are as described in Formula IV or the thirteenth or fourteenth embodiment.

In a sixteenth, the compound of Formula I is selected from one of the following:

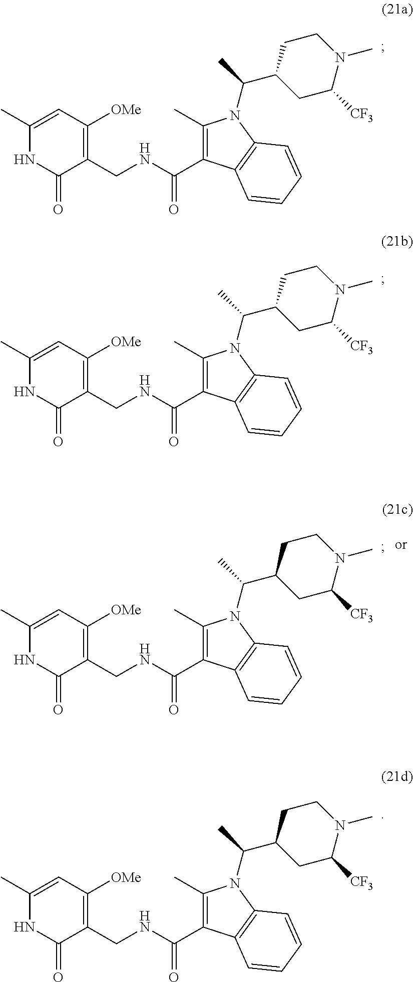

or a pharmaceutically acceptable salt thereof.

In a seventeenth embodiment, the compound of Formula I is a single stereoisomer represented by the following structure:

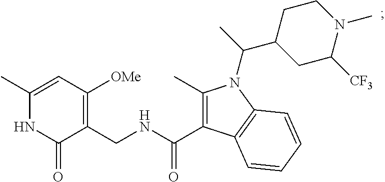

or a pharmaceutically acceptable salt thereof, wherein the stereoisomer has a retention time (RT) of 10.5 min under SFC conditions A (Column: Chiralpak AD-H Mobile phase A: Supercritical CO₂ Mobile phase B: gradient 5-40% iPrOH with 0.05% diethylamine) and 6.7 min under SFC conditions B (Column: Chiralpak OD-H Mobile phase A: Supercritical CO₂ Mobile phase B: gradient 5-40% EtOH with 0.05% diethylamine). Single stereoisomer means greater than 90%, 91%, 92%, 93%, 94%, 95%, 96%, 97%, 98%, or 99% stereomerically pure, stereomerically pure is the weight of the single stereoisomer divided by the total weight of all the stereoisomers represented by the immediately preceding formula.

Also provided is a composition comprising a single stereoisomer represented by the following structure:

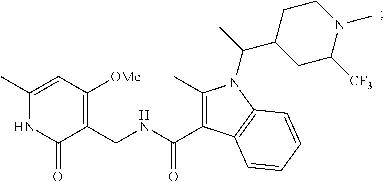

or a pharmaceutically acceptable salt thereof, wherein the stereoisomer has a retention time (RT) of 10.5 min under SFC conditions A (Column: Chiralpak AD-H Mobile phase A: Supercritical CO₂ Mobile phase B: gradient 5-40% iPrOH with 0.05% diethylamine) and 6.7 min under SFC conditions B (Column: Chiralpak OD-H Mobile phase A: Supercritical CO₂ Mobile phase B: gradient 5-40% EtOH with 0.05% diethylamine). Single stereoisomer means greater than 90%, 91%, 92%, 93%, 94%, 95%, 96%, 97%, 98%, or 99% stereomerically pure, wherein stereomerically pure is the weight of the single stereoisomer divided by the total weight of all the stereoisomers in the composition represented by the immediately preceding formula.

In an eighteenth embodiment, the compound of Formula I is selected from one of the following:

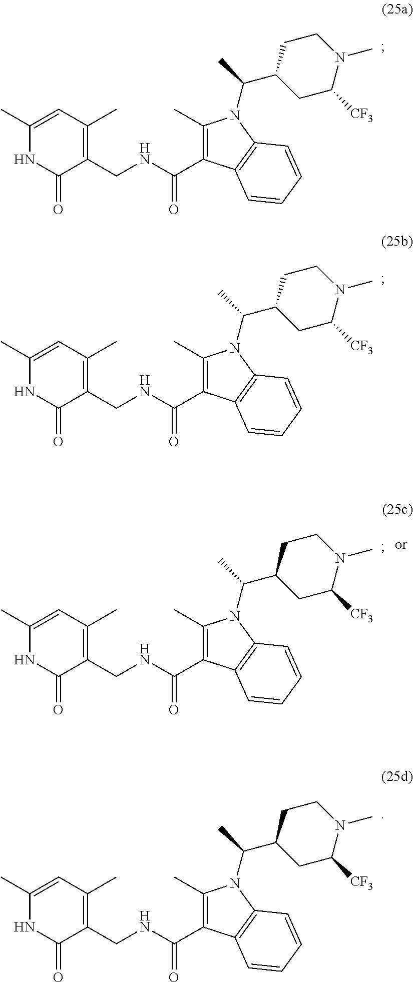

In a nineteenth embodiment, the compound of Formula I is a single stereoisomer represented by the following structure:

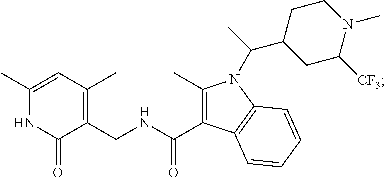

or a pharmaceutically acceptable salt thereof, wherein the stereoisomer has a retention time (RT) of 11.4 min via chiral SFC analysis (Instrument: AD-H Mobile phase A: Supercritical CO₂ Mobile phase B: gradient 5-40% iPrOH with 0.05% diethylamine). Single stereoisomer means greater than 90%, 91%, 92%, 93%, 94%, 95%, 96%, 97%, 98%, or 99% stereomerically pure, stereomerically pure is the weight of the single stereoisomer divided by the total weight of all the stereoisomers represented by the immediately preceding formula.

Also provided is a composition comprising a single stereoisomer represented by the following structure:

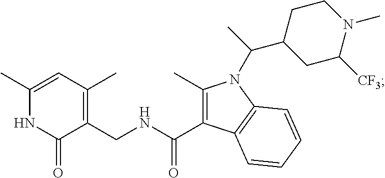

or a pharmaceutically acceptable salt thereof, wherein the stereoisomer has a retention time (RT) of 11.4 min via chiral SFC analysis (Instrument: AD-H Mobile phase A: Supercritical CO₂ Mobile phase B: gradient 5-40% iPrOH with 0.05% diethylamine). Single stereoisomer means greater than 90%, 91%, 92%, 93%, 94%, 95%, 96%, 97%, 98%, or 99% stereomerically pure, stereomerically pure is the weight of the single stereoisomer divided by the total weight of all the stereoisomers in the composition represented by the immediately preceding formula.

Specific examples of compounds are provided in the EXEMPLIFICATION section and are included as part of a twentieth embodiment herein. Pharmaceutically acceptable salts as well as the neutral forms of these compounds are also included.

### 4. Uses, Formulation and Administration

Pharmaceutically Acceptable Compositions

According to another embodiment, this disclosure provides a composition comprising a compound described herein or a pharmaceutically acceptable derivative thereof and a pharmaceutically acceptable carrier, adjuvant, or vehicle. The amount of compound in compositions is such that is effective to measurably modulate a histone methyl modifying enzyme, or a mutant thereof, in a biological sample or in a patient. In certain embodiments, the amount of compound in compositions is such that is effective to measurably modulate a histone methyl modifying enzyme, or a mutant thereof, in a biological sample or in a patient.

In certain embodiments, a composition described herein is formulated for administration to a patient in need of such composition. In some embodiments, a composition described herein is formulated for oral administration to a patient.

The term “patient,” as used herein, means an animal, such as a mammal, and such as a human. The terms “subject” and “patient” may be used interchangeably.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound described herein that, upon administration to a recipient, is capable of providing, either directly or indirectly, a compound described herein or an inhibitorily active metabolite or residue thereof.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions described herein are formulated for oral administration. Such formulations may be administered with or without food. In some embodiments, pharmaceutically acceptable compositions described herein are administered without food. In other embodiments, pharmaceutically acceptable compositions described herein are administered with food.

The amount of compounds described herein that may be combined with the carrier materials to produce a composition in a single dosage form will vary depending upon the host treated and the particular mode of administration. In some embodiments, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Uses of Compounds and Pharmaceutically Acceptable Compositions

Compounds and compositions described herein are generally useful for the modulating of activity of one or more enzymes involved in epigenetic regulation and in particular EZH1 and EZH2 and, even more specifically EZH2 and mutant forms thereof. In some embodiments, compounds described herein down-regulate or suppress the activity of EZH2. In some embodiments, compounds described herein are antagonists of EZH2 activity. In some embodiments, compounds described herein down-regulate or suppress the activity of EZH1. In some embodiments, compounds described herein are antagonists of EZH1 activity.

In some embodiments, compounds and compositions described herein are useful in treating diseases and/or disorders associated with overexpression of EZH1 or EZH2 and/or expression of a mutant form of EZH2, such as those mutant forms that alter EZH2 substrate activity. The study of EZH2 deletions, missense and frameshift mutations suggest that EZH2 functions as a tumor suppressor in blood disorders such as myelodysplastic syndromes (MDS) and myeloid malignancies (Ernst et al., Nat Genet. 2010 August; 42(8):722-6; Nikoloski et al., Nat Genet. 2010 August; 42(8):665-7). In some embodiments, compounds and compositions described herein are useful in treating diseases and/or disorders associated with the presence of EZH2 having a Y641N, Y641C, Y641F, Y641H, Y641S, A677G, or A687 mutation. In a particular aspect of this embodiment, the EZH2 has a Y641N mutation.

In some embodiments, the present disclosure provides a method of treating a subject suffering from a disease and/or disorder associated with overexpression of EZH1 or EZH2 and/or expression of a mutant form of EZH2 comprising the step of administering a compound of Formula I, or a composition comprising any of the foregoing. In some embodiments, the above method additionally comprises the preliminary step of determining if the subject is overexpressing EZH2 or expressing a mutant form of EZH2.

In some embodiments, the disease or disorder associated with the presence of a mutant form of EZH2 is a human B cell lymphoma. In some embodiments, the disease and/or disorder associated with the presence of Y641N EZH2 is follicular lymphoma or diffuse large-B-cell lymphoma. In some embodiments, compounds or compositions described herein are useful in treating blood disorders, such as myelodysplastic syndromes, leukemia, anemia and cytopenia. Sneeringer et al., “Coordinated activities of wild-type plus mutant EZH2 drive tumor-associated hypertrimethylation of lysine 27 on histone H3 (H3K27) in human B-cell lymphomas,” Proceedings of the National Academy of Sciences, PNAS Early Edition published ahead of print on Nov. 15, 2010.

In some embodiments, compounds and compositions described herein are useful in treating diseases and/or disorders associated with cellular proliferation. In some embodiments, compounds and compositions described herein are useful in treating diseases and/or disorders associated with misregulation of cell cycle or DNA repair. In some embodiments, compounds and compositions described herein are useful in treating cancer. Exemplary types of cancer include breast cancer, prostate cancer, colon cancer, renal cell carcinoma, glioblastoma multiforme cancer, bladder cancer, melanoma, bronchial cancer, lymphoma and liver cancer.

In some embodiments, the present disclosure provides a method of reducing the activity of EZH2 in a subject comprising the step of administering a compound of Formula I, or a composition comprising any of the foregoing. In some embodiments, the present disclosure provides a method of reducing the activity of wide-type EZH2 in a subject comprising the step of administering a compound of Formula I, or a composition comprising any of the foregoing. In some embodiments, the present disclosure provides a method of reducing the activity of wild-type EZH1 in a subject comprising the step of administering a compound of Formula I, or a composition comprising any of the foregoing. In some embodiments, the present disclosure provides a method of reducing the activity of a mutant form of EZH2 in a subject comprising the step of administering a compound of Formula I, or a composition comprising any of the foregoing. In some embodiments, the present disclosure provides a method of reducing the activity of a mutant form of EZH2 in a subject comprising the step of administering a compound of Formula I or a composition comprising any of the foregoing, wherein the mutant form of EZH2 is selected from Y641N, Y641C, Y641F, Y641H, Y641S, A677G, or A687V EZH2. Each of these mutations alter the EZH2 substrate activity, and thus facilitate the conversion from a di- to a tri-methylated K27 state. In a more specific aspect, the present disclosure provides a method of reducing the activity of a mutant form of EZH2 in a subject comprising the step of administering a compound of Formula I, or a composition comprising any of the foregoing, wherein the mutant form of EZH2 is Y641N EZH2.

In some embodiments, the present disclosure provides a method of treating a subject suffering from a disease and/or disorder associated with EZH2, wherein the method additionally comprises the preliminary step of determining if the subject is expressing a mutant form of EZH2, such as Y641N, Y641C, Y641F, Y641H, Y641S, A677G, or A687V EZH2. In some embodiments, the present disclosure provides a method of reducing the activity of a mutant form of EZH2, such as Y641N, Y641C, Y641F, Y641H, Y641S, A677G, or A687V EZH2, in a subject in need thereof comprising the step of administering a compound of Formula I, or a composition comprising any of the foregoing. In some embodiments, the present disclosure provides a method of treating a subject suffering from a disease and/or disorder associated with EZH2, wherein the method additionally comprises the preliminary step of determining if the subject has increased levels of histone H3 Lys-27-specific trimethylation (H3K27me3), as compared to a subject known not to express a mutant form of EZH2.

Certain exemplary provided compounds, e.g., having structural formula I are set forth in the EXEMPLIFICATION section below. In some embodiments, a provided compound is one or more compounds selected from those exemplified in the EXEMPLIFICATION section below, or a pharmaceutically acceptable salt thereof.

### EXEMPLIFICATION

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples that follow and the references to the scientific and patent literature cited herein. It should further be appreciated that the contents of those cited references are incorporated herein by reference to help illustrate the state of the art.

General Synthetic Scheme:

As depicted in the Examples below, in certain exemplary embodiments, compounds are prepared according to the following general procedures. It will be appreciated that, although the synthetic methods and Schemes depict the synthesis of certain compounds of the present invention, the following methods and other methods known to one of ordinary skill in the art can be applied to all compounds and subclasses and species of each of these compounds, as described herein.

It will be appreciated that for compound preparations described herein, when reverse phase HPLC is used to purify a compound, a compound may exist as an acid addition salt. In some embodiments, a compound may exist as a formic acid or mono-, di-, or tri-trifluoroacetic acid salt.

It will further be appreciated that the present disclosure contemplates individual compounds described herein. Where individual compounds exemplified are isolated and/or characterized as a salt, for example, as a trifluoroacetic acid salt, the present disclosure contemplates a free base of the salt, as well as other pharmaceutically acceptable salts of the free base.

Unless otherwise noted, all solvents, chemicals, and reagents were obtained commercially and used without purification. The ¹H NMR spectra were obtained in CDCl₃, d₆-DMSO, CD₃OD, or d₆-acetone at 25° C. at 300 MHz on an OXFORD (Varian) with chemical shift (δ, ppm) reported relative to TMS as an internal standard. HPLC-MS chromatograms and spectra were obtained with Shimadzu LC-MS-2020 system. Chiral analysis and purification were obtained with Yilite P270.

### Preparation of Intermediates

### Synthesis of 1-(2-(((4-methoxybenzyl)oxy)methyl)pyridin-4-yl)ethan-1-amine

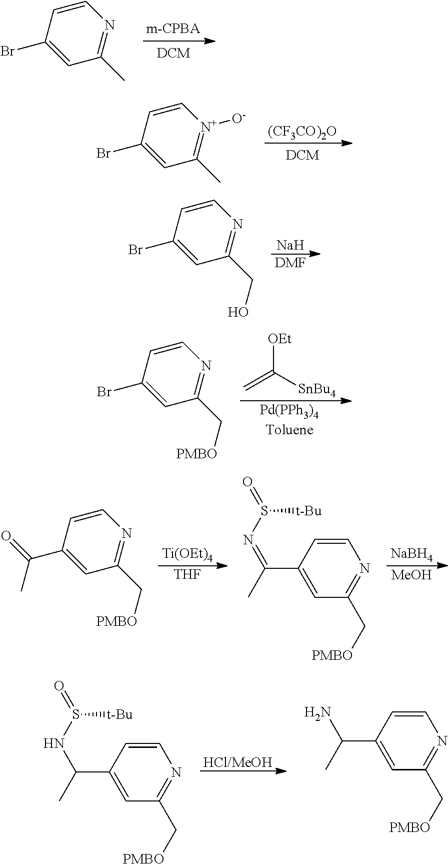

### Step 1: Synthesis of 5-methylnicotinonitrile

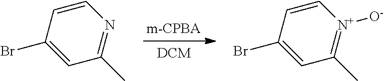

To a solution of 4-bromo-2-methylpyridine 1-oxide (10 g, 0.058 mol) in dichloromethane (150 mL) was added 3-chlorobenzoperoxoic acid (12.08 g, 0.07 mol) in portions at 0° C. The reaction mixture was stirred for 16 hr. TLC showed the starting material was consumed completely and saturated NaHCO₃ solution (100 mL*2) was added. The organic layer was separated, dried over anhydrous Na₂SO₄, filtered and concentrated to afford crude product (9.8 g, crude), which was used in the next step without further purification.

### Step 2: Synthesis of (4-bromopyridin-2-yl)methanol

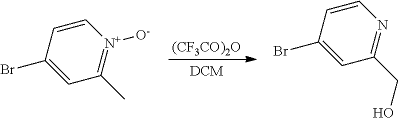

2,2,2-trifluoroacetic anhydride (34 mL, 244 mmol) was slowly added to a solution of 4-bromo-2-methylpyridine 1-oxide (9.8 g, 0.052 mol) in dry dichloromethane (100 mL) at 0° C. under N₂. The resulting solution was refluxed for 16 hr before cooling to room temperature. The volatiles were removed to afford a dark yellow oil. The pH was adjusted to 12-13 with saturated sodium hydroxide solution and the resulting solution was stirred for 16 hr. The mixture was extracted with dichloromethane (100 mL*6). The combined organics layer was washed with brine, dried over anhydrous Na₂SO₄, filtered, and concentrated. The crude residue was purified by silica gel chromatography (PE:EA=5:1 to 1:1) to afford (4-bromopyridin-2-yl)methanol (5 g, 51% yield) as a yellow solid.

### Step 3: Synthesis of 4-bromo-2-(((4-methoxybenzyl)oxy)methyl)pyridine

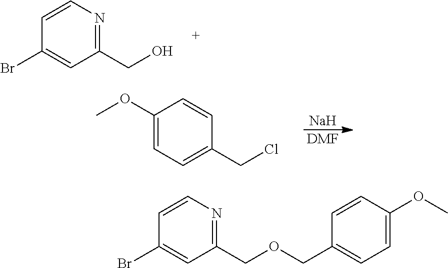

To a solution of (4-bromopyridin-2-yl)methanol (5 g, 26.6 mmol) in anhydrous DMF (100 mL) was added sodium hydride (60% in mineral oil, 1.28 g, 31.9 mmol) in portions at 0° C. After one hour, 1-(chloromethyl)-4-methoxybenzene (5 g, 31.9 mmol) was added dropwise and the mixture was stirred for 16 hr at ambient temperature. The mixture was cooled to 0° C. and quenched by saturated ammonium chloride solution. The aqueous layer was extracted with dichloromethane (100 mL*2). The combine organics layer was washed with brine, dried over anhydrous Na₂SO₄, filtered and concentrated to afford crude residue. The crude residue was purified by silica gel chromatography (PE:EA=10:1 to 5:1) to afford 4-bromo-2-(((4-methoxybenzyl)oxy)methyl)pyridine (7 g, 85.4% yield).

### Step 4: Synthesis of 1-(2-(((4-methoxybenzyl)oxy)methyl)pyridin-4-yl)ethanone

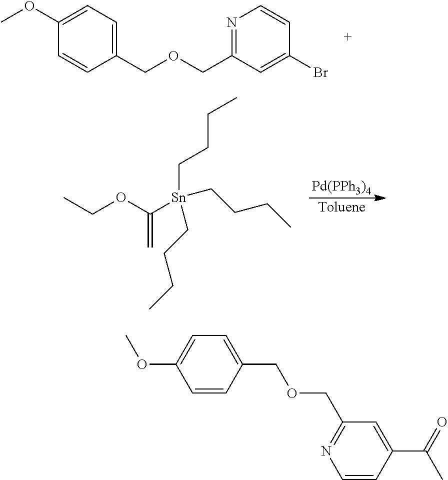

To a mixture of 4-bromo-2-[(4-methoxyphenyl)methoxymethyl]pyridine (7 g, 22.7 mmol) and tributyl(1-ethoxyvinyl)stannane (10.65 g, 29.5 mmol) in toluene (80 mL) was added tetrakis(triphenylphosphine) palladium (524 mg, 454 umol) in one portion at r.t. under N₂. The mixture was heated to 110° C. and stirred for 3 hr. TLC showed the reaction was complete. The mixture was cooled to room temperature and concentrated. The residue was treated with 10% hydrochloric acid solution and stirred for one hour. After addition of 1N sodium hydroxide solution, the mixture was stirred for 16 hr. The aqueous layer was extracted with EA (200 mL*2). The combined organics layer was washed with saturated brine (200 mL), dried over anhydrous Na₂SO₄, filtered and concentrated. The crude residue was purified by silica gel chromatography (PE/EA=5/1-2/1) to afford 1-[2-[(4-methoxyphenyl)methoxymethyl]-4-pyridyl]ethanone (5.00 g, 18.43 mmol, 81.1% yield) as a yellow oil.

### Step 5: Synthesis of (R)—N-(1-(2-(((4-methoxybenzyl)oxy)methyl)pyridin-4-yl)ethylidene)-2-methylpropane-2-sulfinamide

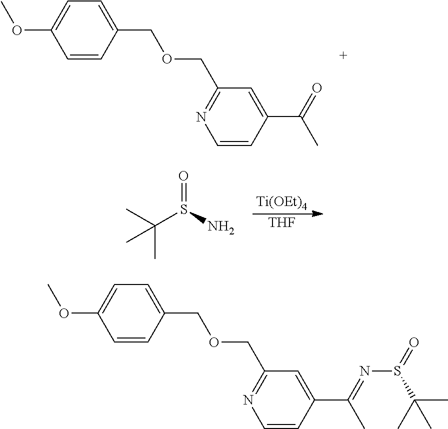

To a mixture of 1-[2-[(4-methoxyphenyl)methoxymethyl]-4-pyridyl]ethanone (5.00 g, 18.43 mmol) and (R)-2-methylpropane-2-sulfinamide (2.90 g, 23.96 mmol) in THF (100 mL) was added tetraethoxytitanium (8.41 g, 36.86 mmol) in one portion at room temperature under N₂. The mixture was heated to 70° C. and stirred for 16 hr. TLC showed the reaction was complete. The mixture was cooled to room temperature and concentrated under reduced pressure. The residue was poured into water (150 mL) and the mixture was stirred for 30 min. The mixture was filtered and the filtrate was extracted with EA (200 mL*2). The combined organics layer was washed with saturated brine (200 mL), dried over anhydrous Na₂SO₄, filtered and concentrated. The crude residue was purified by silica gel chromatography (PE/EA=3/1, 2/1) to afford (R)—N-[1-[2-[(4-methoxyphenyl)methoxymethyl]-4-pyridyl]ethylidene]-2-methyl-propane-2-sulfinamide (5.40 g, 14.42 mmol, 78.24% yield) as a yellow oil.

### Step 6: Synthesis of N-(1-(2-(((4-methoxybenzyl)oxy)methyl)pyridin-4-yl)ethyl)-2-methylpropane-2-sulfinamide

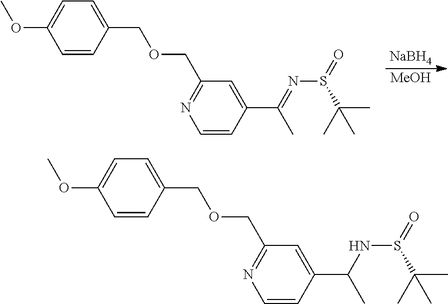

To a mixture of (R)—N-[1-[2-[(4-methoxyphenyl)methoxymethyl]-4-pyridyl]ethylidene]-2-methyl-propane-2-sulfinamide (5.40 g, 14.42 mmol) in MeOH (50 mL) was added NaBH₄ (1.64 g, 43.26 mmol) in portions at 0° C. under N₂. The mixture was stirred at room temperature for 2 hr. TLC showed the reaction was complete. The mixture was poured into ice-water (w/w=1/1) (150 mL) and stirred for 10 min. The aqueous phase was extracted with EA (200 mL*2). The combined organics phase was washed with saturated brine (100 mL*2), dried over anhydrous Na₂SO₄, filtered, and concentrated to afford N-[1-[2-[(4-methoxyphenyl)methoxymethyl]-4-pyridyl]ethyl]-2-methyl-propane-2-sulfinamide (5.43 g, 14.42 mmol, crude) which was used for the next step without purification.

### Step 7: Synthesis of 1-(2-(((4-methoxybenzyl)oxy)methyl)pyridin-4-yl)ethanamine

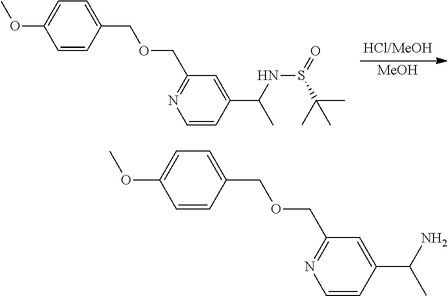

To a mixture of N-[1-[2-[(4-methoxyphenyl)methoxymethyl]-4-pyridyl]ethyl]-2-methyl-propane-2-sulfinamide (5.43 g, 14.42 mmol) in MeOH (40 mL) was added HCl (10 mL, 40.00 mmol, 4M in MeOH) in one portion at room temperature under N₂. The mixture was stirred for 1 hr. TLC showed the reaction was complete. The mixture was concentrated and the residue was poured into water (100 mL). The pH was adjusted to 9-10 with 1N sodium hydroxide solution. The aqueous phase was extracted with EA (200 mL*3). The combined organics phase was washed with saturated brine (200 mL), dried over anhydrous Na₂SO₄, filtered and concentrated to afford 1-[2-[(4-methoxyphenyl)methoxymethyl]-4-pyridyl]ethanamine (3.50 g, 12.85 mmol) was used for next step without purification.

### Synthesis of 1-(quinolin-4-yl)ethan-1-amine

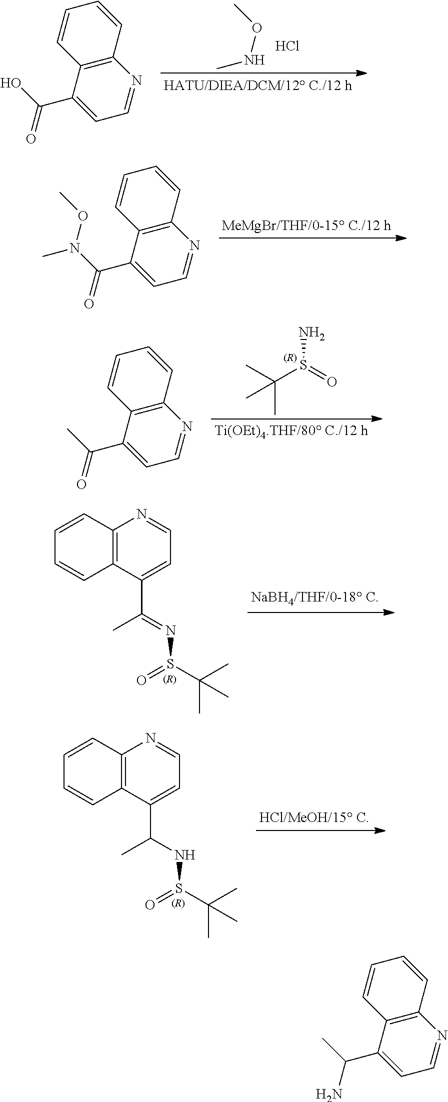

### Step 1: Synthesis of N-methoxy-N-methylquinoline-4-carboxamide

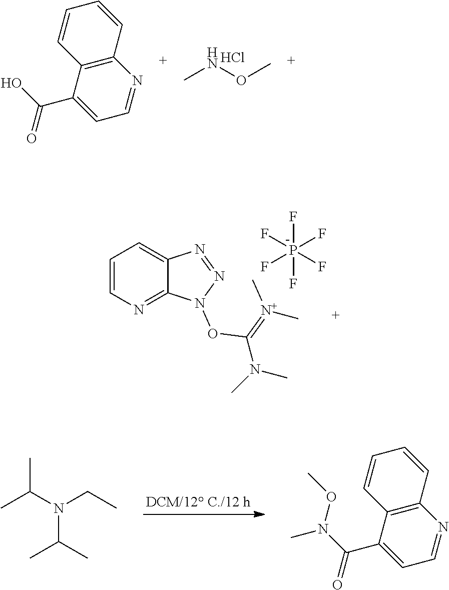

To a stirred solution of quinoline-4-carboxylic acid (12.00 g, 69.30 mmol) in anhydrous dichloromethane (50 ml) was added 2-(3H-[1,2,3]triazolo[4,5-b]pyridin-3-yl)-1,1,3,3-tetramethylisouronium hexafluorophosphate(V) (39.52 g, 103.95 mmol). The mixture solution was stirred for 15 minutes at room temperature (15° C.), and then N-ethyl-N-isopropylpropan-2-amine (26.87 g, 207.89 mmol) and N,O-dimethylhydroxylamine hydrochloride (8.1 g, 83.16 mmol) were slowly added to the reaction solution. The reaction solution was stirred for 12 hr at room temperature. The reaction solution was quenched by addition of water (50 ml) and concentrated. The aqueous solution was extracted with ethyl acetate (50 mL×3). The combined organics layer was dried over sodium sulfate, filtered and concentrated to afford the crude N-methoxy-N-methylquinoline-4-carboxamide (14.98 g). It was used in next step directly.

### Step 2: Synthesis of 1-(quinolin-4-yl)ethanone

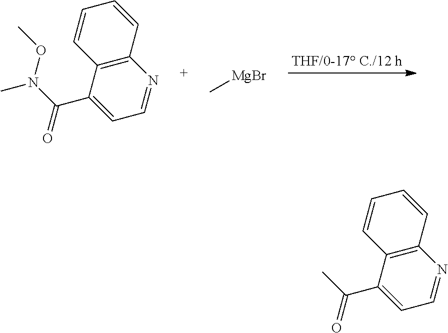

To a stirred solution of N-methoxy-N-methylquinoline-4-carboxamide (14.98 g, 69.28 mmol) in anhydrous tetrahydrofuran (50 mL) was slowly added methylmagnesium bromide (23.1 mL, 3.0 M in diethyl ether) at 0° C. under N₂. The mixture was stirred for 12 hr at 17° C. The reaction was quenched by addition of saturated ammonium chloride solution, and then extracted with ethyl acetate (50 ml×3). The combined organics layer was dried over sodium sulfate, filtered, and concentrated. The crude residue was purified by column chromatography on silica gel (petroleum ether:ethyl acetate=1:1) to afford 1-(quinolin-4-yl)ethanone (6 g, 50.59%). LCMS m/z 172 [M+H]⁺.

### Step 3: Synthesis of (R)-2-methyl-N-(1-(quinolin-4-yl)ethylidene)propane-2-sulfinamide

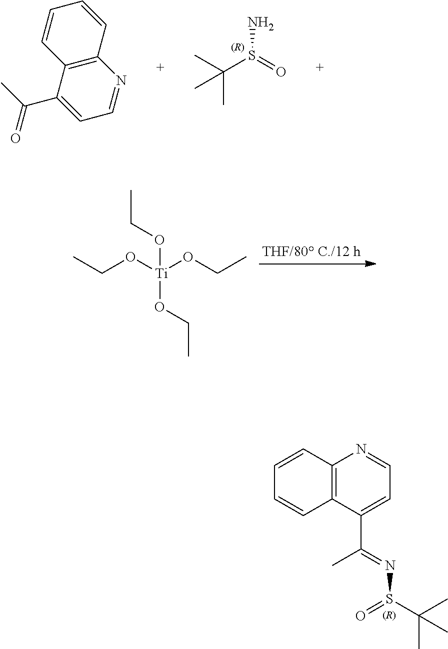

To a stirred solution of 1-(quinolin-4-yl)ethanone (6 g, 35.05 mmol) and (R)-2-methylpropane-2-sulfinamide (8.50 g, 70.1 mmol) in anhydrous tetrahydrofuran (30 mL) was added tetraethoxytitanium (31.98 g, 140.2 mmol) at room temperature (20° C.). The mixture was refluxed for 12 hr under N₂. The reaction solution was quenched by addition of water (30 mL) and then stirred for another 1 hr at room temperature. The solid was filtered out and the filtrate was extracted with ethyl acetate (30 ml×3). The combined organics layer was dried over sodium sulfate, filtered, and concentrated. The crude residue was purified by column chromatography on silica gel (petroleum ether:ethyl acetate=5:1˜1:1) to afford (R)-2-methyl-N-(1-(quinolin-4-yl)ethylidene)propane-2-sulfinamide (7.79 g, 81%). LCMS m/z 275 [M+H]⁺.

### Step 4: Synthesis of (R)-2-methyl-N-(1-(quinolin-4-yl)ethyl)propane-2-sulfinamide

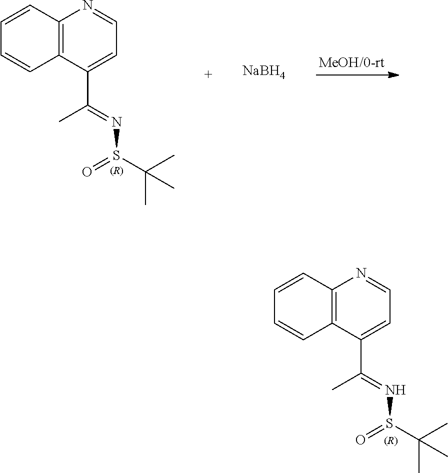

To a stirred solution of (R)-2-methyl-N-(1-(quinolin-4-yl)ethylidene)propane-2-sulfinamide (6 g, 21.87 mmol) in anhydrous methanol (20 mL) was slowly added sodium borohydride (2.48 g, 65.60 mmol) at 0° C. The mixture was warmed to room temperature and stirred for 4 hr. The reaction solution was quenched by addition of water (20 mL) and then concentrated. The aqueous solution was extracted with ethyl acetate (20 ml×3). The combined organics layer was dried over sodium sulfate, filtered, and concentrated to afford the mixture stereomers of (R)-2-methyl-N-(1-(quinolin-4-yl)ethyl)propane-2-sulfinamide (5.8 g, 95.96%). LCMS m/z 277 [M+H]⁺.

### Step 5: Synthesis of 1-(quinolin-4-yl)ethan-1-amine

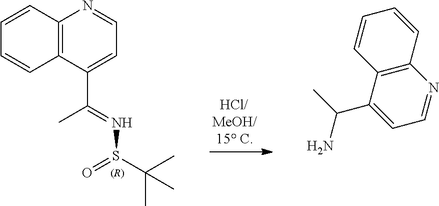

(R)-2-methyl-N-(1-(quinolin-4-yl)ethyl)propane-2-sulfinamide (5.8 g, 20.98 mmol) was dissolved hydrochloric acid solution (15 mL, 4M in MeOH). The mixture was stirred for 3 hr at room temperature. The reaction solution was concentrated and the residue triturated with methyl tert-butyl ether (10 mL×3), and ethyl acetate (10 mL×3). The solid was dissolved in 20 mL water and the aqueous solution was neutralized by addition of saturated sodium hydrogen carbonate solution. This solution was then extracted with ethyl acetate (15 mL×3). The combined organics layer was dried over sodium sulfate, filtered, and concentrated to afford 1-(quinolin-4-yl)ethan-1-amine (1.2 g). This material was used without further purification.

### Synthesis of 1-(2-(difluoromethyl)pyridin-4-yl)ethanone

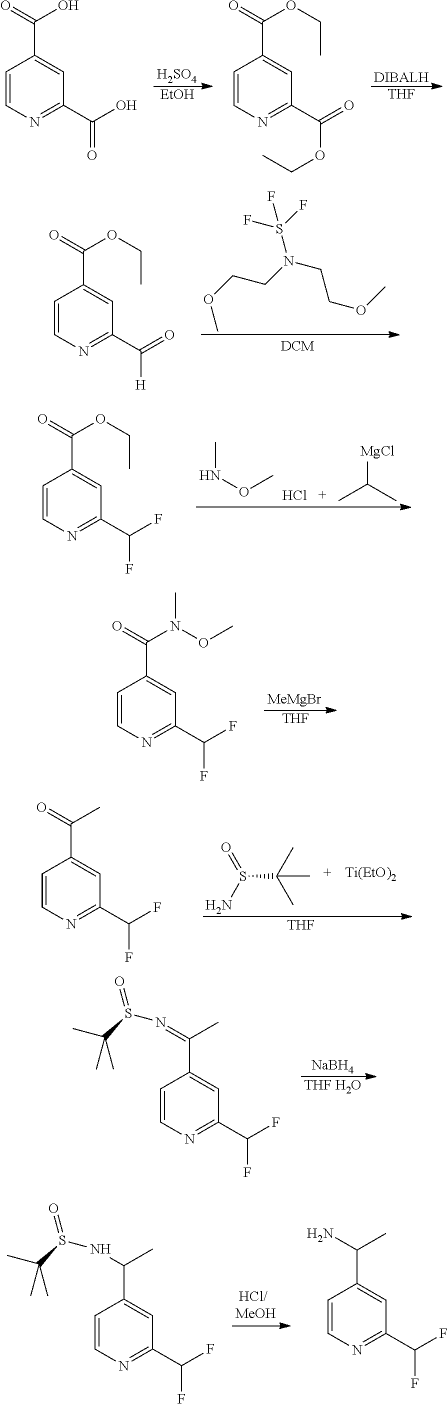

### Step 1: Synthesis of diethyl pyridine-2,4-dicarboxylate

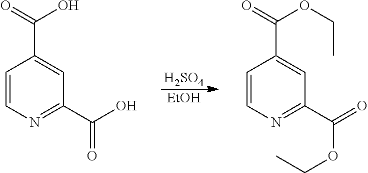 [SMILES: "EtOH" ; O=C(O)c1ccnc(C(=O)O)c1 ; "H2SO4" ; CCOC(=O)c1ccnc(C(=O)OCC)c1]

To a solution of pyridine-2,4-dicarboxylic acid (50.0 g, 270 mmol) in anhydrous ethanol (800 mL) was added sulfuric acid (40 mL) drop-wise at 0° C. After addition completed, the mixture was refluxed overnight. After the reaction was complete, the mixture was cooled to room temperature and concentrated. The residue was basified to pH=8 with sodium hydrogencarbonate solution and then extracted with ethyl acetate (500 mL×3). The combined organics layer was dried over anhydrous sodium sulfate and concentrated to afford crude diethyl pyridine-2,4-dicarboxylate which was used directly in subsequent steps.

### Step 2: Synthesis of ethyl 2-formylisonicotinate

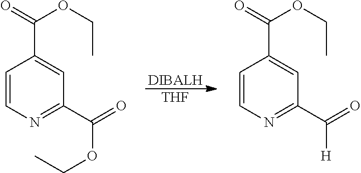

To a solution of diethyl pyridine-2,4-dicarboxylate (20.0 g, 89.6 mmol) in anhydrous tetrahydrofuran (300 mL) was added diisobutylaluminum hydride (134.4 mL, 134.4 mmol, 1 mol/L in toluene) dropwise at −78° C. After the addition was complete, the mixture was stirred at −78° C. for 3 hr and then poured slowly into an ice-cold mixture of acetic acid (50 mL) and water (250 mL). The mixture was then warmed to room temperature and stirred for 1.5 hr. The pH of the mixture was adjusted to 8 with sodium hydrogencarbonate solution at 0° C. and extracted with ethyl acetate (500 mL×5). The combined organics layer was dried over anhydrous sodium sulfate and concentrated. The crude residue was purified by column chromatography on silica gel (Petroleum ether: ethyl acetate=40:1˜10:1) to afford ethyl 2-formylisonicotinate (13.0 g, yield 81%). LCMS m/z 180 [M+H]⁺. ¹H NMR (400 MHz, CDCl3) δ 1.45-1.42 (t, 3H), 4.48-4.43 (m, 2H), 8.10-8.09 (d, J=4.4 Hz, 1H), 8.48 (s, 1H), 8.95-8.94 (d, J=4.8 Hz, 1H), 10.15 (s, 1H).

### Step 3: Synthesis of ethyl 2-(difluoromethyl)isonicotinate

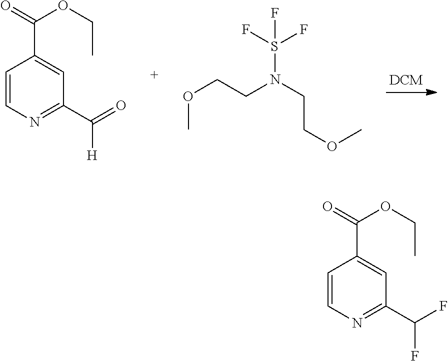

To a solution of ethyl 2-formylisonicotinate (7.0 g, 39 mmol) in anhydrous dichloromethane (220 mL) was added bis(2-methoxyethyl)amino sulfur trifluoride (17.1 g, 78 mmol) dropwise at 0° C. After addition the mixture was stirred at room temperature for 5 hr. The mixture was then neutralized by sodium hydrogencarbonate solution at 0° C. and extracted with dichloromethane (200 mL×3). The combined organics layer was dried over anhydrous sodium sulfate and concentrated. The crude residue was purified by column chromatography on silica gel (Petroleum ether:ethyl acetate=30:1˜15:1) to afford ethyl 2-(difluoromethyl)isonicotinate (5.8 g, yield 73.8%). LCMS m/z 202 [M+H]⁺.

¹H NMR (400 MHz, CDCl3) δ 1.44-1.40 (t, 3H), 4.47-4.41 (m, 2H), 6.83-6.55 (t, 1H), 7.97-7.61 (d, J=4.8 Hz, 1H), 8.18 (s, 1H), 10.15 (s, 1H), 8.81-8.80 (d, J=5.2 Hz, 1H).

### Step 4: Synthesis of 2-(difluoromethyl)-N-methoxy-N-methylisonicotinamide

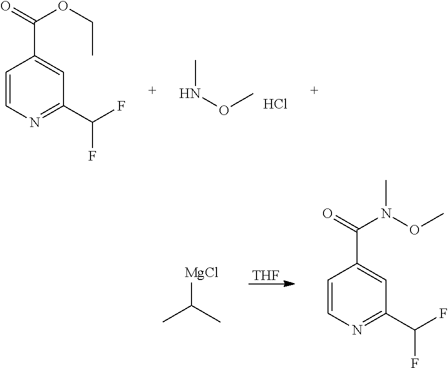

To a solution of ethyl 2-(difluoromethyl)isonicotinate (5.8 g, 29 mmol) and N,O-dimethylhydroxylamine hydrochloride (7.0 g, 72.2 mmol) in anhydrous tetrahydrofuran (70 mL) was added isopropylmagnesium chloride (73 mL, 146 mmol, 2 mol/L) dropwise at −15° C. The mixture was stirred at 0° C. for 1.5 hr before the reaction was quenched with ammonium chloride solution at 0° C. and extracted with ethyl acetate (200 mL×3). The combined organics layer was dried over anhydrous sodium sulfate and concentrated. The crude residue was purified by column chromatography on silica gel (Petroleum ether: ethyl acetate=10:1˜4:1) to afford 2-(difluoromethyl)-N-methoxy-N-methylisonicotinamide (2.7 g, yield 43.2%). LCMS m/z 217 [M+H]⁺.

### Step 5: Synthesis of 1-(2-(difluoromethyl)pyridin-4-yl)ethanone

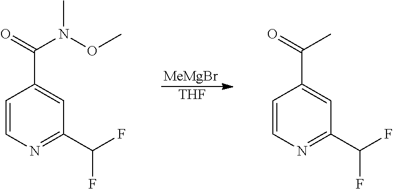

To a solution of 2-(difluoromethyl)-N-methoxy-N-methylisonicotinamide (2.7 g, 12.5 mmol) in anhydrous tetrahydrofuran (40 mL) was added methylmagnesium bromide (13 ml, 39 mmol, 3 mol/L) dropwise at 0° C. The mixture was stirred at 0° C. for 3 hr before the reaction was quenched with water at 0° C. and extracted with ethyl acetate (100 mL×3). The combined organics layer was dried over anhydrous sodium sulfate and concentrated to afford crude 1-(2-(difluoromethyl)pyridin-4-yl)ethanone.

¹H NMR (400 MHz, CDCl3) δ 2.67 (s, 3H), 6.86-6.58 (m, 1H), 7.86-7.85 (d, J=4.4 Hz, 1H), 8.06 (s, 1H), 8.86-8.45 (d, J=5.2 Hz, 1H).

### Step 6: Synthesis of (R)—N-(1-(2-(difluoromethyl)pyridin-4-yl)ethylidene)-2-methylpropane-2-sulfinamide

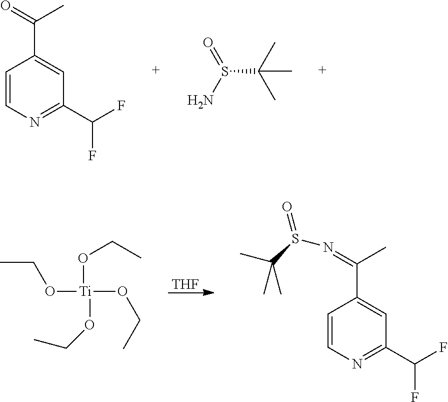

To a solution of 1-(2-(difluoromethyl)pyridin-4-yl)ethanone (1.8 g, 10.5 mmol) in anhydrous tetrahydrofuran (300 mL) was added (R)-2-methylpropane-2-sulfinamide (1.9 g, 15.75 mmol) and tetraethoxytitanium (4.78 g, 21 mmol) at room temperature. The mixture was stirred at 80° C. overnight. The mixture was quenched with water and extracted with ethyl acetate (100 mL×3). The combined organics layer was dried over anhydrous sodium sulfate and concentrated. The crude residue was purified by column chromatography on silica gel (Petroleum ether:ethyl acetate=30:1˜4:1) to afford (R)—N-(1-(2-(difluoromethyl)pyridin-4-yl)ethylidene)-2-methylpropane-2-sulfinamide (2.5 g, 9.11 mmol, yield 86.8%) as a brown oil.

### Step 7: Synthesis of (R)—N-(1-(2-(difluoromethyl)pyridin-4-yl)ethyl)-2-methylpropane-2-sulfinamide

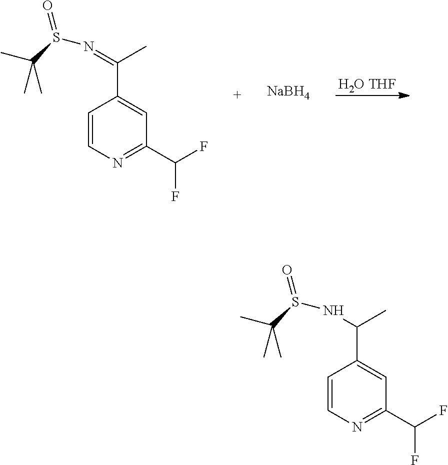 [SMILES: C/C(=N/[S@](=O)C(C)(C)C)c1ccnc(C(F)F)c1 ; "NaBH4" ; CC(N[S@](=O)C(C)(C)C)c1ccnc(C(F)F)c1 ; "H2O THF"]

To a solution of (R)—N-(1-(2-(difluoromethyl)pyridin-4-yl)ethylidene)-2-methylpropane-2-sulfinamide (2.5 g, 9.1 mmol) in tetrahydrofuran (38 mL) and water (1 mL) was added sodium borohydride (1.03 g, 27.4 mmol) at −78° C. The mixture was warmed to 0° C. stirred for 4 hr. The reaction was quenched with water at 0° C. and extracted with ethyl acetate (150 mL×3). The combined organics layer was dried over anhydrous sodium sulfate and concentrated to afford crude (R)—N-(1-(2-(difluoromethyl)pyridin-4-yl)ethyl)-2-methylpropane-2-sulfinamide which was used in subsequent steps without further purification.

### Step 8: Synthesis of 1-(2-(difluoromethyl)pyridin-4-yl)ethanamine

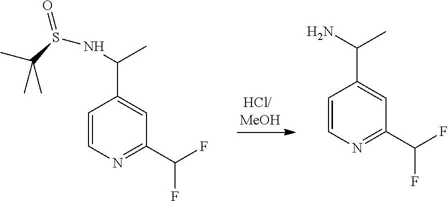 [SMILES: CC(N[S@](=O)C(C)(C)C)c1ccnc(C(F)F)c1 ; CC(N)c1ccnc(C(F)F)c1]

To a solution of (R)—N-(1-(2-(difluoromethyl)pyridin-4-yl)ethyl)-2-methylpropane-2-sulfinamide (2.0 g, 7.3 mmol) in methanol (20 mL) was added hydrogen chloride (6 mL, 4M in MeOH). The mixture was stirred at room temperature for 4 hr. The reaction was concentrated the residue dissolved in water and washed with ethyl acetate (50 mL×2). The water layer was basified to pH=8 with sodium carbonate solution and extracted with dichloromethane (200 mL×4). The combined DCM layer was dried over anhydrous sodium sulfate and concentrated to afford crude 1-(2-(difluoromethyl)pyridin-4-yl)ethanamine.

### Synthesis of 1-(3-(trifluoromethyl)pyridin-4-yl)ethanamine

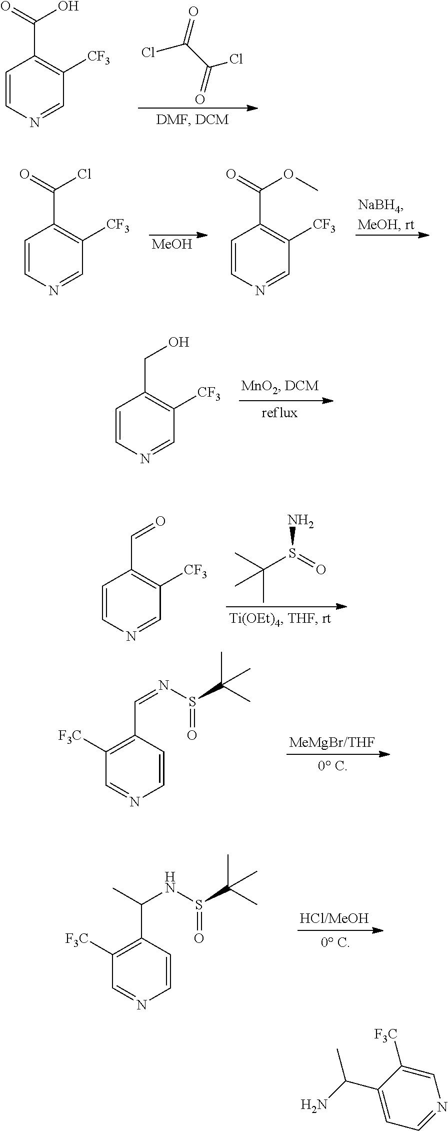

### Step 1: Synthesis of 3-(trifluoromethyl)isonicotinoyl chloride

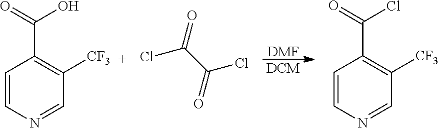

To a solution of 3-(trifluoromethyl)isonicotinic acid (1 g, 5.23 mmol) in dichloromethane (20 mL) was added oxalyl chloride (996 mg, 7.85 mmol) at 0° C. DMF (0.1 mL) was added. The resulting mixture was stirred for 1 hours at room temperature (25-30° C.). The reaction mixture was concentrated to afford crude 3-(trifluoromethyl)isonicotinoyl chloride (1.2 g)

### Step 2: Synthesis of methyl 3-(trifluoromethyl)isonicotinate

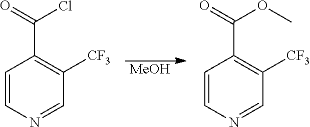

A solution of 3-(trifluoromethyl)isonicotinoyl chloride (1.1 g, 5.25 mmol) in methanol (10 mL) was stirred for 1 hours at room temperature (25-30° C.). The reaction mixture was concentrated and the residue was dissolved in DCM and then washed with water. The organic layer was dried over sodium sulfate and then evaporated to afford crude methyl 3-(trifluoromethyl)isonicotinate (600 mg, yield 55.9%).

### Step 3: Synthesis of (3-(trifluoromethyl)pyridin-4-yl)methanol

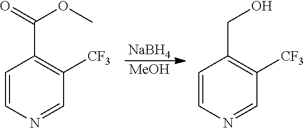

To a solution of methyl 3-(trifluoromethyl)isonicotinate (600 mg, 2.92 mmol) in methanol (15 mL) was added sodium borohydride (664 mg, 17.55 mmol) at 0° C. The resulting mixture was then warmed to room temperature and stirred for 16 hr. The reaction mixture was then concentrated and the residue was dissolved in DCM and washed with water. The organic layer was dried over sodium sulfate and concentrated to afford crude (3-(trifluoromethyl)pyridin-4-yl)methanol (520 mg, yield 100%).

### Step 4: Synthesis of 3-(trifluoromethyl)isonicotinaldehyde

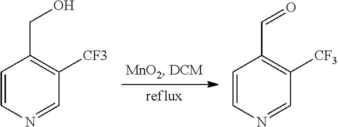 [SMILES: OCc1ccncc1C(F)(F)F ; O=Cc1ccncc1C(F)(F)F]

To a solution of (3-(trifluoromethyl)pyridin-4-yl)methanol (450 mg, 2.54 mmol) in DCM (20 mL) was added manganese(IV) oxide (2.21 g, 25.4 mmol) at 45° C. The resulting mixture was stirred for 8 hr at 45° C. The reaction mixture was filtered and the filtrate concentrated to afford crude 3-(trifluoromethyl)isonicotinaldehyde (400 mg, yield 89.9%).

### Step 5: Synthesis of 2-methyl-N-((3-(trifluoromethyl)pyridin-4-yl)methylene)propane-2-sulfinamide

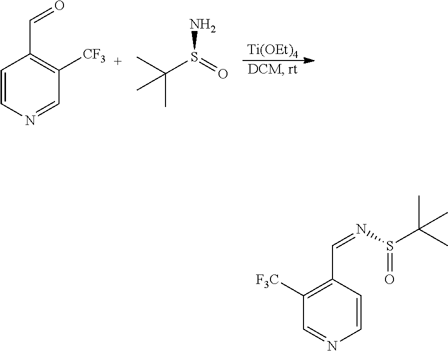

To a solution of 3-(trifluoromethyl)isonicotinaldehyde (300 mg, 1.71 mmol) in DCM (10 mL) was added (S)-2-methylpropane-2-sulfinamide (249 mg, 2.06 mmol) and tetraethoxytitanium (430 mg, 1.88 mmol) at 25° C. The resulting mixture was stirred for 4 hours at 25° C. Water (1.2 mL) was added and then the solution was dried over sodium sulfate. The reaction mixture was filtered and the filtrate was evaporated to afford crude (S)-2-methyl-N-((3-(trifluoromethyl)pyridin-4-yl)methylene)propane-2-sulfinamide (450 mg, yield 94.3%).

### Step 6: Synthesis of 2-methyl-N-(1-(3-(trifluoromethyl)pyridin-4-yl)ethyl)propane-2-sulfinamide

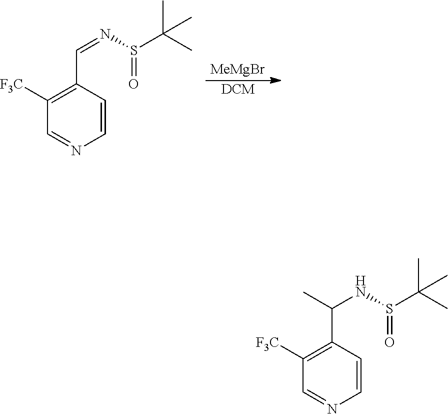

To a solution of (S)-2-methyl-N-((3-(trifluoromethyl)pyridin-4-yl)methylene)propane-2-sulfinamide (550 mg, 1.98 mmol) in DCM (10 mL) was added MeMgBr (2.58 mL, 7.71 mmol) at 0° C. The resulting mixture was stirred for 16 hr at room temperature. Ammonium chloride solution (10 mL) was added and the mixture extracted with DCM. The organics layer was dried over sodium sulfate and concentrated to afford crude (S)-2-methyl-N-(1-(3-(trifluoromethyl)pyridin-4-yl)ethyl)propane-2-sulfinamide (580 mg, yield 99.5%).

### Step 7: Synthesis of 1-(3-(trifluoromethyl)pyridin-4-yl)ethanamine

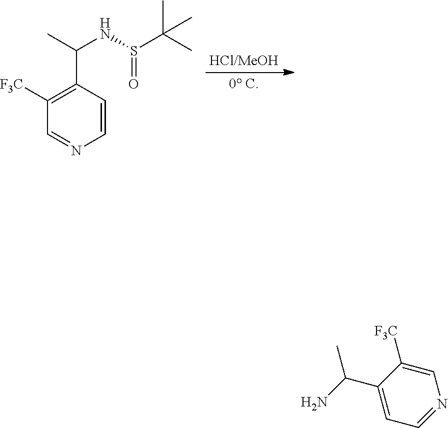 [SMILES: CC(N[S@@](=O)C(C)(C)C)c1ccncc1C(F)(F)F ; CC(N)c1ccncc1C(F)(F)F]

To a solution of (S)-2-methyl-N-(1-(3-(trifluoromethyl)pyridin-4-yl)ethyl)propane-2-sulfinamide (500 mg, 1.7 mmol) in MeOH (5 mL) was added HCl (5 mL, 4 M in MeOH) at 0° C. The resulting mixture was stirred for 3 hr at 25° C. The reaction mixture was concentrated to afford crude 1-(3-(trifluoromethyl)pyridin-4-yl)ethanamine (350 mg, yield 100%).

### Synthesis of 1-(2-(4-fluorophenyl)pyridin-4-yl)ethanamine

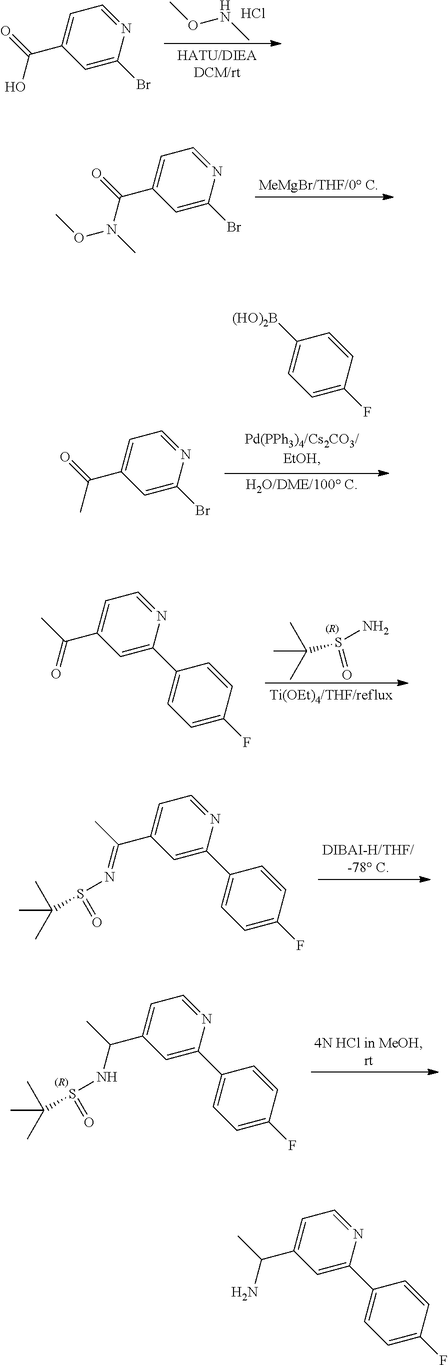

### Step 1: Synthesis of 2-bromo-N-methoxy-N-methylisonicotinamide

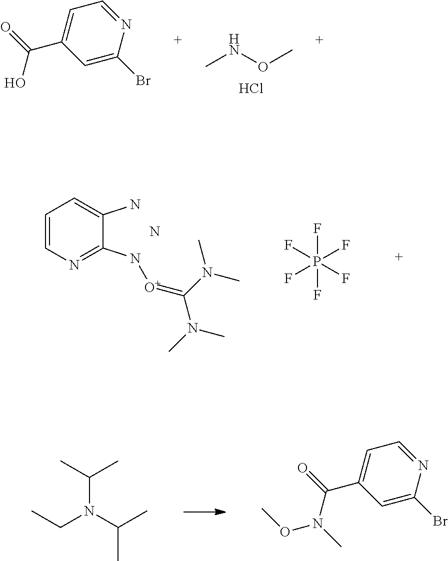

To a solution of 2-bromoisonicotinic acid (20 g, 100 mmol) in dichloromethane (200 mL) was added N-ethyl-N-isopropylpropan-2-amine (38.7 g, 300 mmol) and 2-(3H-[1,2,3]triazolo[4,5-b]pyridin-3-yl)-1,1,3,3-tetramethyluronium hexafluorophosphate(V) (76 g, 200 mmol). The reaction was stirred for 30 min at room temperature before addition of N,O-dimethylhydroxylamine hydrochloride (19 g, 200 mmol). The mixture was stirred at room temperature for 18 hr. The solution was then partitioned between dichloromethane and water, the organic layer was separated, dried over magnesium sulfate, and concentrated. The crude residue was purified by silica gel chromatography (petroleum ether:ethyl acetate=3:1) to give 2-bromo-N-methoxy-N-methylisonicotinamide as a brown oil (21.6 g, 88.5%).

### Step 2: Synthesis of 1-(2-bromopyridin-4-yl)ethanone

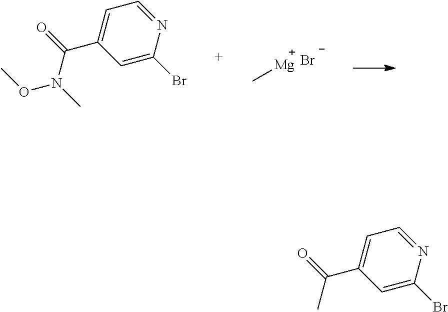

To a solution of 2-bromo-N-methoxy-N-methylisonicotinamide (24.4 g, 100 mmol) in tetrahydrofuran (250 mL) was added methylmagnesium bromide (100 mL, 300 mmol, 3 M in tetrahydrofuran) at 0° C. The reaction mixture was stirred for 2 h. Water was added and the mixture was concentrated. The remaining aqueous solution was extracted with ethyl acetate. The organics layer was washed with brine, dried over sodium sulfate, and concentrated. The crude residue was purified by silica gel chromatography (petroleum ether: ethyl acetate=7:1) to afford 1-(2-bromopyridin-4-yl)ethanone as a yellow solid (8 g, 40.2%).

### Step 3: Synthesis of 1-(2-(4-fluorophenyl)pyridin-4-yl)ethanone

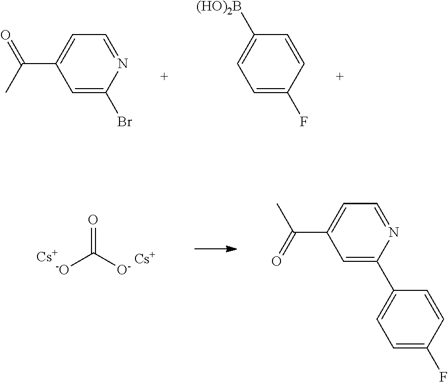

To a solution of 1-(2-bromopyridin-4-yl)ethanone (8 g, 40 mmol) and (4-fluorophenyl)boronic acid (8.4 g, 60 mmol) in ethanol (30 mL), water (30 mL) and 1,2-dimethoxyethane (30 mL) were added cesium carbonate (39 g, 120 mmol) and tetrakis[triphenylphosphine]palladium(0) (2.3 g, 2 mmol) under nitrogen. The reaction mixture was stirred at 90° C. for 16 hr. The solution was cooled to room temperature and filtered. The filtrate was extracted with ethyl acetate and the organic layer was concentrated. The crude product was purified by silica gel chromatography (petroleum ether:ethyl acetate=8:1) to give 1-(2-(4-fluorophenyl)pyridin-4-yl)ethanone as a yellow solid (8 g, 93%).

### Step 4: Synthesis of (R)—N-(1-(2-(4-fluorophenyl)pyridin-4-yl)ethylidene)-2-methylpropane-2-sulfinamide

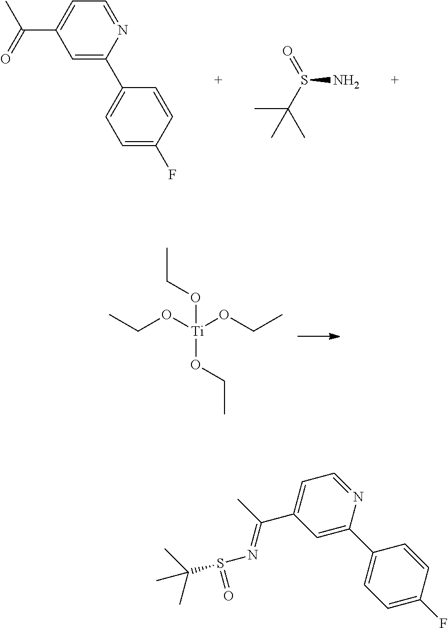

To a solution of 1-(2-(4-fluorophenyl)pyridin-4-yl)ethanone (5 g, 25 mmol) and (R)-2-methylpropane-2-sulfinamide (6 g, 50 mmol) in tetrahydrofuran (100 mL) was added titanium(IV) ethoxide (22.8 g, 100 mmol). The reaction mixture was stirred at 80° C. for 18 hr under nitrogen. The solution was cooled to room temperature and the reaction quenched with water (50 mL). The reaction mixture was extracted with ethyl acetate and the combined organics layer was dried over sodium sulfate and concentrated. The crude residue was purified by silica gel chromatography (petroleum ether:ethyl acetate=6:1) to afford (R)—N-(1-(2-(4-fluorophenyl)pyridin-4-yl)ethylidene)-2-methylpropane-2-sulfinamide as a yellow oil (5.3 g, 66.7%).

### Step 5: Synthesis of (R)—N-(1-(2-(4-fluorophenyl)pyridin-4-yl)ethyl)-2-methylpropane-2-sulfinamide

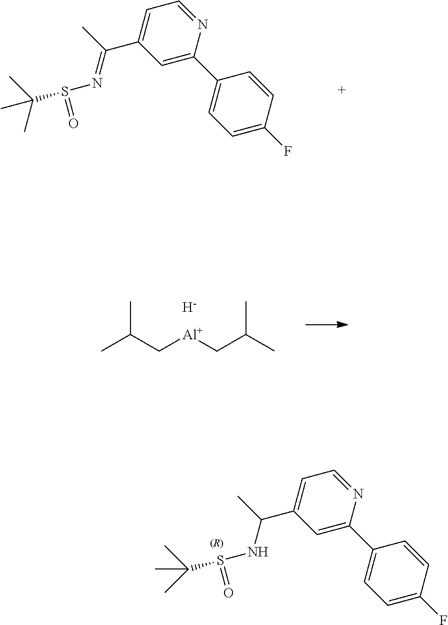

To a solution of (R)—N-(1-(2-(4-fluorophenyl)pyridin-4-yl)ethylidene)-2-methylpropane-2-sulfinamide (4.9 g, 15.4 mmol) in tetrahydrofuran (50 mL) was added diisobutylaluminum hydride (31 mL, 30.8 mmol, 1 M in toluene) at −70° C. The reaction mixture was stirred at −70° C. for 2 hr and then the mixture was quenched by addition of methanol (1 mL). Sodium hydroxide (2 M) was added and the reaction mixture was extracted with ethyl acetate. The combined organics layer was dried over sodium sulfate and concentrated to afford crude (R)—N-(1-(2-(4-fluorophenyl)pyridin-4-yl)ethyl)-2-methylpropane-2-sulfinamide as a yellow oil (4.5 g, 91.3%).

### Step 6: Synthesis of 1-(2-(4-fluorophenyl)pyridin-4-yl)ethanamine

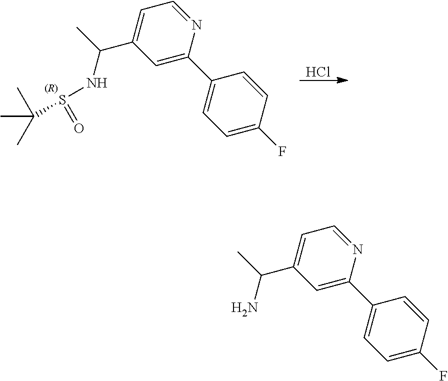

The mixture of (R)—N-(1-(2-(4-fluorophenyl)pyridin-4-yl)ethyl)-2-methylpropane-2-sulfinamide (5 g, 15.6 mmol) and hydrochloric acid (60 mL, 4 M in MeOH) was stirred at room temperature for 3 hr. The solvent was removed and the residue was basified by aqueous sodium hydrogen carbonate. The resulting basic solution was extracted with dichloromethane and the organic layer was concentrated to afford crude 1-(2-(4-fluorophenyl)pyridin-4-yl)ethanamine (3.4 g, crude) as a brown oil.

### Synthesis of 4-(1-aminoethyl)-1-methylpiperidin-2-one

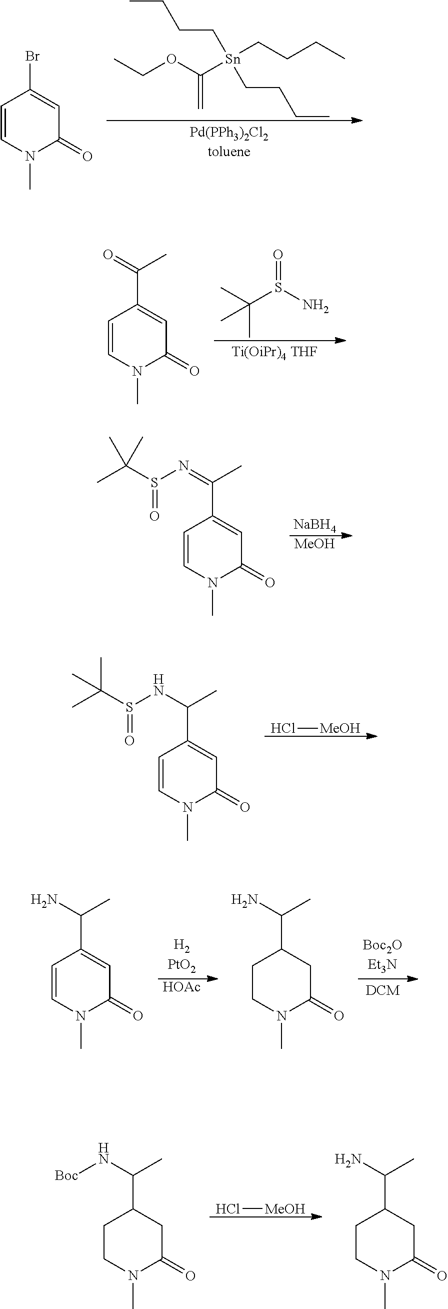

### Step 1: Synthesis of 4-acetyl-1-methylpyridin-2(1H)-one

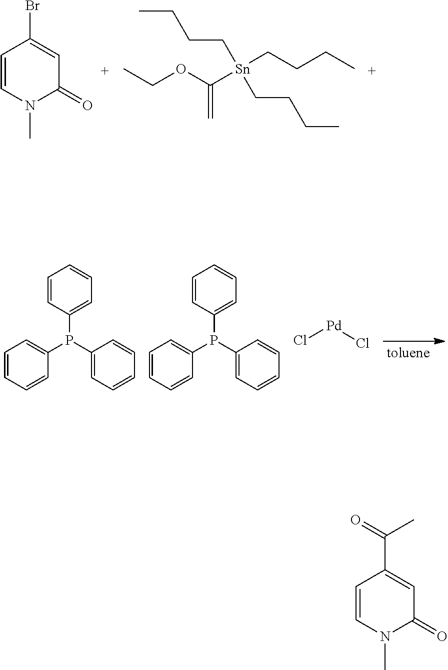

A mixture of 4-bromo-1-methylpyridin-2(1H)-one (4.8 g, 25.5 mmol), tributyl(1-ethoxyvinyl)stannane (10.0 g, 28.05 mmol), and bis(triphenylphosphine) palladium(II) dichloride (180 mg, 0.26 mmol) in toluene (100 ml) was refluxed for 20 hr. The solution was cooled to room temperature and treated with hydrochloric acid (5%) for 30 min before extracting with ethyl acetate (100 ml*8). The organic layers were dried over sodium sulfate and concentrated to afford 4-acetyl-1-methylpyridin-2(1H)-one (2.0 g, 54.5% yield). LCMS m/z 152 [M+H]⁺.

### Step 2: Synthesis of 2-methyl-N-(1-(1-methyl-2-oxo-1,2-dihydropyridin-4-yl)ethylidene)propane-2-sulfinamide

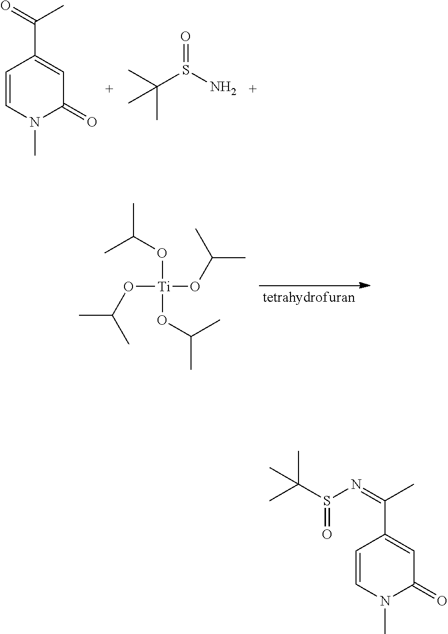

To a solution of 4-acetyl-1-methylpyridin-2(1H)-one (2.0 g, 13.24 mmol) and 2-methylpropane-2-sulfinamide (2.4 g, 19.87 mmol) in tetrahydrofuran (100 ml) was added tetraethoxytitanium (4.58 g, 15.89 mmol) at 0° C. The mixture was heated to 60° C. and stirred for 20 hr. The solution was cooled to room temperature and water (3 ml) was added followed by magnesium sulfate. The slurry was stirred for 1 h at room temperature before being filtered. The filtrate was concentrated under reduced pressure to afford 2-methyl-N-(1-(1-methyl-2-oxo-1,2-dihydropyridin-4-yl)ethylidene)propane-2-sulfinamide (2.5 g, 74.4% yield). LCMS m/z 255 [M+H]+.

### Step 3: Synthesis of 2-methyl-N-(1-(1-methyl-2-oxo-1,2-dihydropyridin-4-yl)ethyl)propane-2-sulfinamide

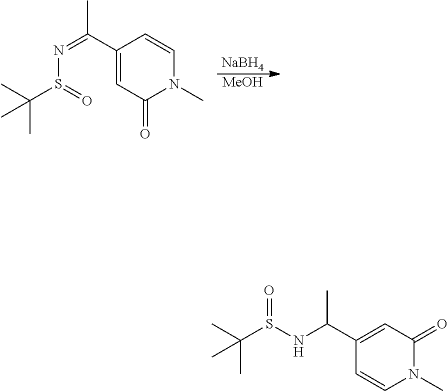 [SMILES: "MeOH" ; C/C(=N/S(=O)C(C)(C)C)c1ccn(C)c(=O)c1 ; "NaBH4" ; CC(NS(=O)C(C)(C)C)c1ccn(C)c(=O)c1]

To a solution of 2-methyl-N-(1-(1-methyl-2-oxo-1,2-dihydropyridin-4-yl)ethylidene) propane-2-sulfinmide (2.5 g, 9.84 mmol) dissolved in methanol (100 mL) was added sodium borohydride (3.73 g, 98.4 mmol) at 0° C. in portions. The mixture was stirred at room temperature for 3 hr. The mixture was quenched with saturated ammonium chloride solution (50 ml) and extracted with dichloromethane (100 ml*4). The organics layer was dried over sodium sulfate and concentrated to afford N-(1-(1-methyl-2-oxo-1,2-dihydropyridin-4-yl) ethyl) propane-2-sulfinamide (2.4 g, 95.2% yield). LCMS m/z 257 [M+H]⁺.

### Step 4: Synthesis of 4-(1-aminoethyl)-1-methylpyridin-2(1H)-one

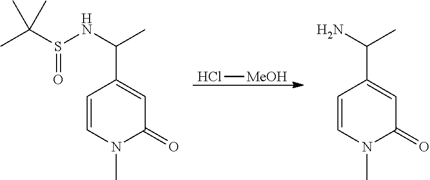

A solution of N-(1-(1-methyl-2-oxo-1,2-dihydropyridin-4-yl) ethyl)propane-2-sulfinamide (2.4 g, 9.37 mmol) dissolved in methanolic hydrogen chloride solution (100 ml, 4M) was stirred at room temperature for 1.5 hr. The mixture was concentrated to afford 4-(1-aminoethyl)-1-methylpyridin-2(1H)-one (2.0 g, 100% yield)

### Step 5: Synthesis of 4-(1-aminoethyl)-1-methylpiperidin-2-one

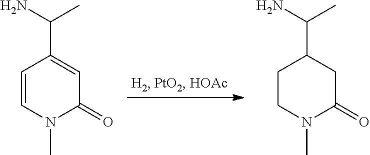

A mixture of 4-(1-aminoethyl)-1-methylpyridin-2(1H)-one (1.8 g, 11.84 mmol) and platinum dioxide (700 mg, 3.09 mmol) dissolved in acetic acid (40 mL) was stirred at room temperature under a hydrogen atmosphere (45 Psi) for 15 hr. The mixture was filtered and the filtrate was concentrated to afford 4-(1-aminoethyl)-1-methylpiperidin-2-one (1.4 g, 76.1% yield)

### Step 6: Synthesis of tert-butyl (1-(1-methyl-2-oxopiperidin-4-yl)ethyl)carbamate

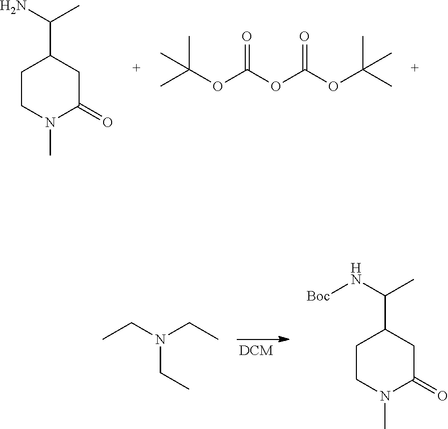

A mixture of 4-(1-aminoethyl)-1-methylpiperidin-2-one (1.4 g, 8.97 mmol), di-tert-butyl dicarbonate (3.91 g, 17.92 mmol) and triethylamine (2.72 g, 26.88 mmol) dissolved in dichloromethane (30 mL) was stirred at room temperature for 3 hr. The mixture was washed with water and purified by column chromatography to afford tert-butyl (1-(1-methyl-2-oxopiperidin-4-yl) ethyl) carbamate (800 mg, 34.8% yield)

### Step 7: Synthesis of 4-(1-aminoethyl)-1-methylpiperidin-2-one

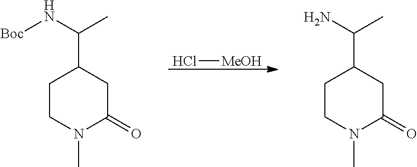

A solution of tert-butyl (1-(1-methyl-2-oxopiperidin-4-yl)ethyl)carbamate (0.8 g, 3.13 mmol) dissolved in methanolic hydrogen chloride solution (20 ml, 4 M) was stirred at room temperature for 1.5 hr. The mixture was concentrated to afford 4-(1-aminoethyl)-1-methylpiperidin-2-one (0.55 g, 91.2% yield). 11-1 NMR (400 MHz, METHANOL-d4) δ 3.52-3.44 (m, 2H), 3.28 (br. s., 1H), 3.01 (d, J=3.0 Hz, 3H), 2.63-2.49 (m, 1H), 2.34-2.21 (m, 1H), 2.14 (br. s., 1H), 2.05 (t, J=11.7 Hz, 1H), 1.74-1.58 (m, 1H), 1.34 (t, J=6.1 Hz, 3H)

### General Procedure for Indole Formation

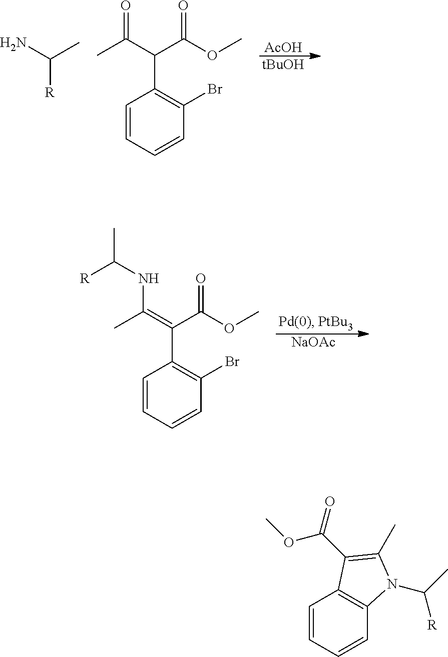

Step 1: Enamine Formation

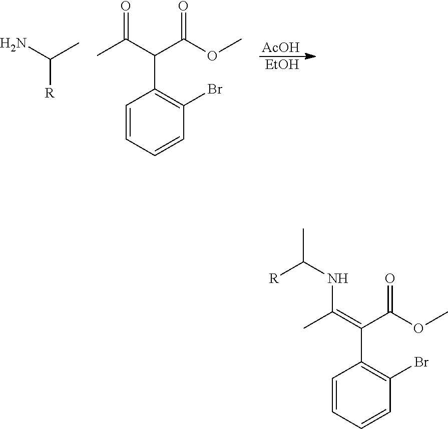

To a round bottom flask containing a magnetic stir bar was charged a solution of methyl 2-(2-bromophenyl)-3-oxobutanoate (1.0 eq) in EtOH (0.3-0.5 M), amine (1.2 equiv), and AcOH (1.2 equiv). The reaction vessel was fitted with a reflux condenser and subsequently heated to reflux for 18 h. After 18 h, the reaction mixture was cooled room temperature and the EtOH was removed in vacuo. The resultant oil material was partitioned between saturated aqueous NaHCO₃ and EtOAc. The layers were separated and the aqueous phase was extracted with EtOAc (2×). The combined organic phases were washed with water, dried with MgSO₄, and concentrated in vacuo to afford an oil. This resultant oil was purified via silica gel chromatography using ethyl acetate/hexanes mixtures as eluent to afford the enamine (typically 85%-97% yield).

Step 2: Palladium-Catalyzed Indole Cyclization

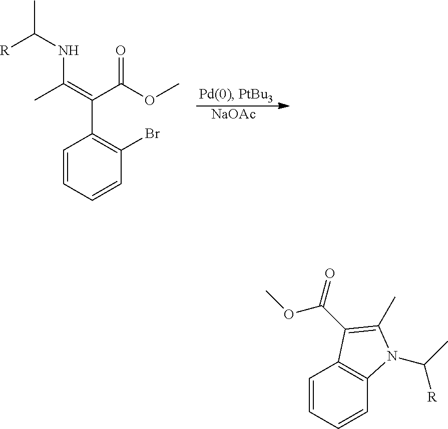

To a mixture of enamine (1.0 eq) in 1,4-dioxane (0.1-0.3 M) were added Pd₂(dba)₃ (0.1 eq), AcONa (3.0 eq), and tri-tert-butylphosphonium tetrafluoroborate (0.2 eq) in one portion under N₂. The mixture was stirred at 110° C. until complete (typically 3-16 hr). The mixture was filtered and the he filter cake was washed with EtOAc (2×) and the filtrate was concentrated. The crude residue was purified via silica gel chromatography typically using hexanes/ethyl acetate mixtures as eluent to afford the title compound (typically 80%-95% yield).

The following indole intermediates were synthesized following the general procedure outlined above using the appropriate amine starting material and methyl 2-(2-bromophenyl)-3-oxobutanoate. For a synthetic procedure for methyl 2-(2-bromophenyl)-3-oxobutanoate, see: Vaswani, R. G., et al. A Practical Synthesis of Indoles via a Pd-Catalyzed C—N Ring Formation. Org. Lett. 2014, 16, 4114-4117.

| | | LCMS m/z |
|---|---|---|
| Name | Structure | [M + H]⁺ |
| methyl 1-(1-(2-(((4- methoxybenzyl)oxy)methyl)pyri din-4-yl)ethyl)-2-methyl-1H- indole-3-carboxylate | 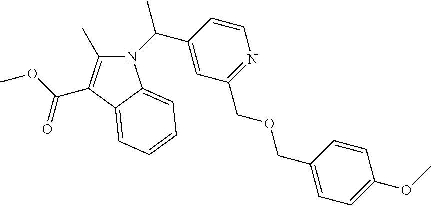 | |
| methyl 2-methyl-1-(1-(quinolin- 4-yl)ethyl)-1H-indole-3- carboxylate | 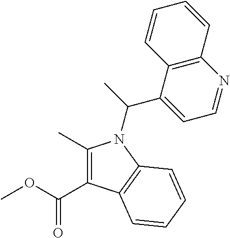 [SMILES: COC(=O)c1c(C)n(C(C)c2ccnc3ccccc23)c2ccccc12] | 345 |
| methyl 1-(1-(2- (difluoromethyl)pyridin-4- yl)ethyl)-2-methyl-1H-indole-3- carboxylate | 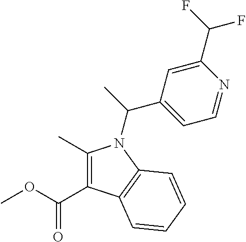 [SMILES: COC(=O)c1c(C)n(C(C)c2ccnc(C(F)F)c2)c2ccccc12] | |
| methyl 2-methyl-1-(1-(3- (trifluoromethyl)pyridin-4- yl)ethyl)-1H-indole-3- carboxylate | 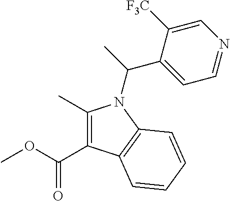 [SMILES: COC(=O)c1c(C)n(C(C)c2ccncc2C(F)(F)F)c2ccccc12] | 363 |
| methyl 1-(1-(2-(4- fluorophenyl)pyridin-4-yl)ethyl)- 2-methyl-1H-indole-3- carboxylate | 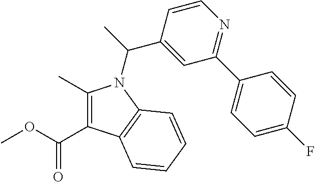 [SMILES: COC(=O)c1c(C)n(C(C)c2ccnc(-c3ccc(F)cc3)c2)c2ccccc12] | |
| methyl 2-methyl-1-(1-(1-methyl- 2-oxopiperidin-4-yl)ethyl)-1H- indole-3-carboxylate | 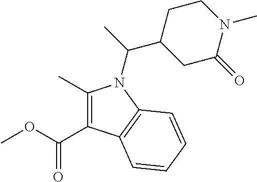 | 351 [N + Na]⁺ |

### Preparation of Compounds of Formula I

### Example 1: Synthesis of N-((4-methoxy-6-methyl-2-oxo-1,2-dihydropyridin-3-yl)methyl)-2-methyl-1-(1-(1-methyl-2-oxopiperidin-4-yl)ethyl)−1H-indole-3-carboxamide (11)

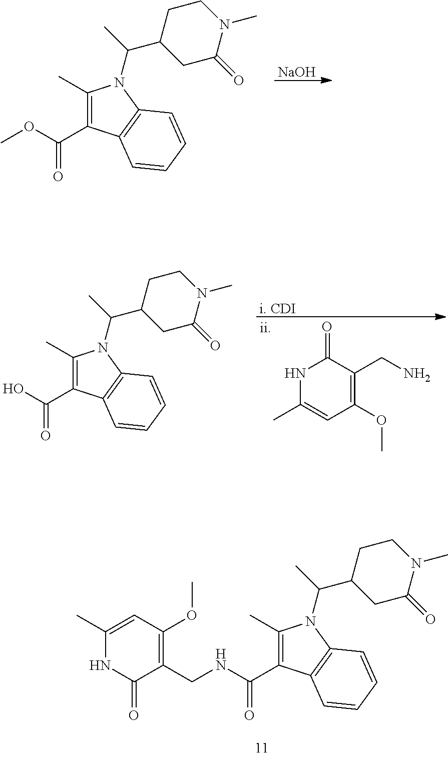

### Step 1: Synthesis of 2-methyl-1-(1-(1-methyl-2-oxopiperidin-4-yl)ethyl)-1H-indole-3-carboxylic Acid

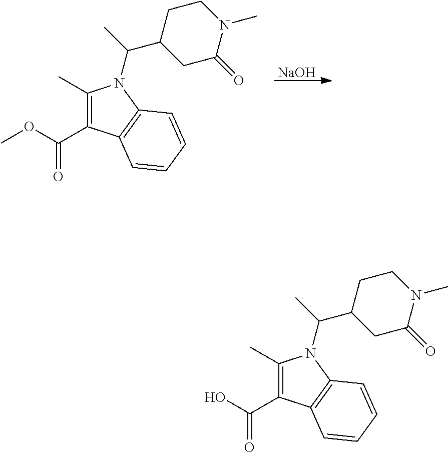

To a resealable vial was added methyl 2-methyl-1-(1-(1-methyl-2-oxopiperidin-4-yl)ethyl)-1H-indole-3-carboxylate (447 mg, 1.37 mmol), EtOH (6 mL), and sodium hydroxide (1.14 mL, 6.85 mmol). The vial was sealed and heated to reflux overnight. The solution was then cooled to room temperature and diluted with water. The pH was adjusted to 4-5 with 1N HCl and extracted with EtOAc. The combined organics layer was washed with water, brine, dried over Na₂SO₄, filtered, and concentrated to afford crude 2-methyl-1-(1-(1-methyl-2-oxopiperidin-4-yl)ethyl)-1H-indole-3-carboxylic acid (0.42 g, 1.34 mmol).

### Step 2: Synthesis of N-((4-methoxy-6-methyl-2-oxo-1,2-dihydropyridin-3-yl)methyl)-2-methyl-1-(1-(1-methyl-2-oxopiperidin-4-yl)ethyl)-1H-indole-3-carboxamide

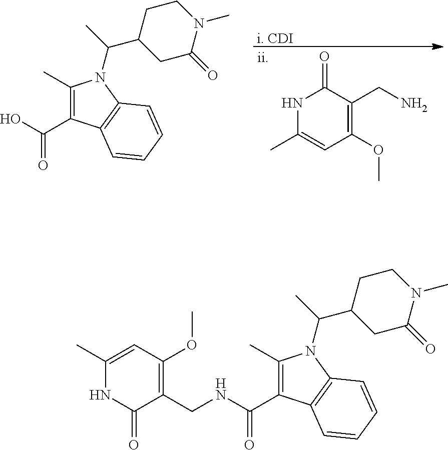

To a round bottomed flask was added 2-methyl-1-(1-(1-methyl-2-oxopiperidin-4-yl)ethyl)-1H-indole-3-carboxylic acid (425 mg, 1.37 mmol), THF, and CDI (0.33 g, 2.06 mmol). The solution was heated to reflux for 1 h before cooling to room temperature and addition of 3-(aminomethyl)-4-methoxy-6-methylpyridin-2(1H)-one (0.46 g, 2.74 mmol). The solution was heated to 65° C. overnight before cooling to room temperature and diluting with water and EtOAc. The layers were separated and the aqueous was extracted with EtOAc. The combined organics were washed with water, brine, dried over Na₂SO₄, filtered, and concentrated. The crude residue was purified on silica gel to afford N-((4-methoxy-6-methyl-2-oxo-1,2-dihydropyridin-3-yl)methyl)-2-methyl-1-(1-(1-methyl-2-oxopiperidin-4-yl)ethyl)-1H-indole-3-carboxamide (0.12 g, 0.26 mmol). LCMS m/z 465 [M+H]⁺.

### Example 2: Synthesis of 1-(1-(2-(hydroxymethyl)-1-methylpiperidin-4-yl)ethyl)-N-((4-methoxy-6-methyl-2-oxo-1,2-dihydropyridin-3-yl)methyl)-2-methyl-1H-indole-3-carboxamide Hydrochloride (30)

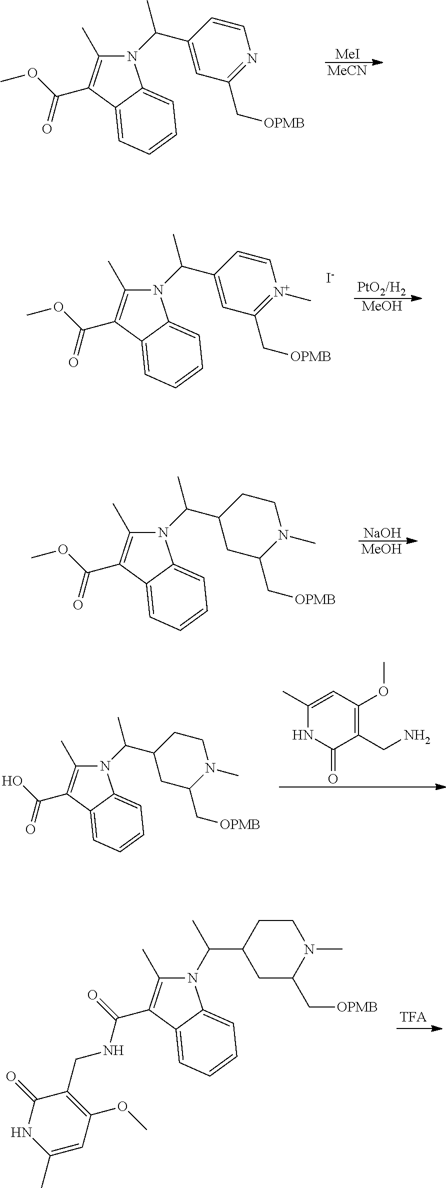

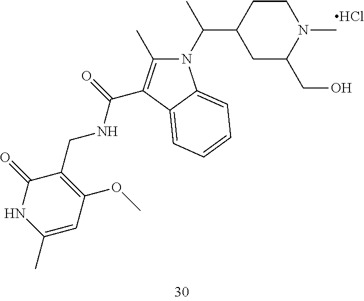 [SMILES: COc1cc(C)[nH]c(=O)c1CNC(=O)c1c(C)n(C(C)C2CCN(C)C(CO)C2)c2ccccc12 ; Cl]

### Step 1: Synthesis of 2-(((4-methoxybenzyl)oxy)methyl)-4-(1-(3-(methoxycarbonyl)-2-methyl-1H-indol-1-yl)ethyl)-1-methylpyridin-1-ium Iodide

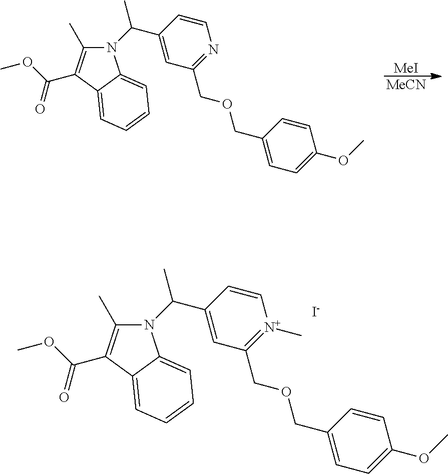

To a mixture of methyl 1-(1-(2-(((4-methoxybenzyl)oxy)methyl)pyridin-4-yl)ethyl)-2-methyl-1H-indole-3-carboxylate (751.24 mg, 1.69 mmol) in MeCN (10 mL) was added methyl iodide (24 g, 16.9 mmol) in one portion under N₂. The mixture was stirred at 80° C. for 3 hr. LCMS showed the reaction was complete. The mixture was cooled to room temperature and concentrated under reduced pressure to afford 2-(((4-methoxybenzyl)oxy)methyl)-4-(1-(3-(methoxycarbonyl)-2-methyl-1H-indol-1-yl)ethyl)-1-methylpyridin-1-ium iodide (800.00 mg, crude) which was used for next step without purification.

### Step 2: Synthesis of methyl 1-(1-(2-(((4-methoxybenzyl)oxy)methyl)-1-methylpiperidin-4-yl)ethyl)-2-methyl-1H-indole-3-carboxylate

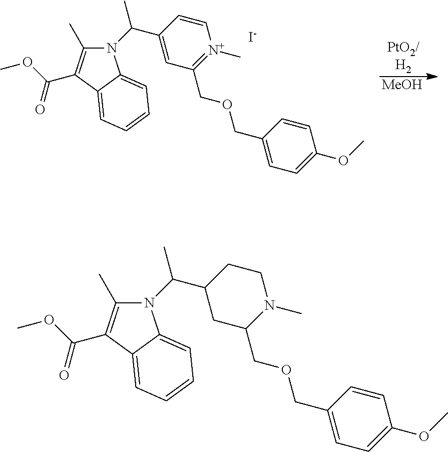

To a solution of 2-(((4-methoxybenzyl)oxy)methyl)-4-(1-(3-(methoxycarbonyl)-2-methyl-1H-indol-1-yl)ethyl)-1-methylpyridin-1-ium iodide (400.00 mg, 680.89 umol) in MeOH (6 mL) and HOAc (1 mL) was added PtO₂ (30.92 mg, 136.18 umol) under N₂. The suspension was degassed under vacuum and purged with H₂ several times. The mixture was stirred under H₂ (50 PSI) for 16 hr. LCMS showed the starting material was completely consumed. The reaction mixture was filtered and the filtrate was concentrated to afford methyl 1-(1-(2-(((4-methoxybenzyl)oxy)methyl)-1-methylpiperidin-4-yl)ethyl)-2-methyl-1H-indole-3-carboxylate (320.00 mg) which was used for next step without purification.

### Step 3: Synthesis of 1-(1-(2-(((4-methoxybenzyl)oxy)methyl)-1-methylpiperidin-4-yl)ethyl)-2-methyl-1H-indole-3-carboxylic Acid

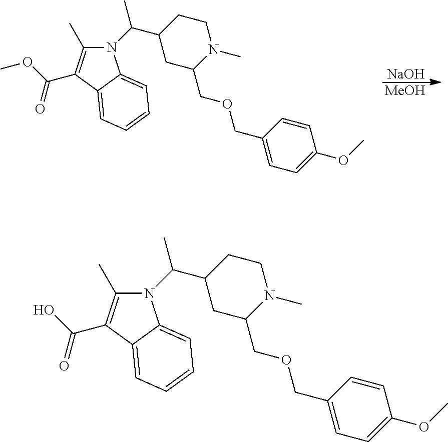

To a mixture of methyl 1-[1-[2-[(4-methoxyphenyl)methoxymethyl]-1-methyl-4-piperidyl]ethyl]-2-methyl-indole-3-carboxylate (320.00 mg, 688.77 umol) in H₂O (4 mL) and MeOH (16 mL) was added NaOH (551.02 mg, 13.78 mmol) in one portion under N₂. The mixture was stirred at 80° C. for 16 hr. TLC showed the reaction was complete. The mixture was cooled to room temperature and concentrated under reduced pressure. The residue was poured into water (50 mL) and the pH was adjusted to 5-6 with 1N hydrochloric acid solution. The aqueous phase was extracted with DCM (100 mL*2). The combined organics phase was washed with saturated brine (100 mL), dried over anhydrous Na₂SO₄, filtered and concentrated to afford 1-[1-[2-[(4-methoxyphenyl)methoxymethyl]-1-methyl-4-piperidyl]ethyl]-2-methyl-indole-3-carboxylic acid (310.00 mg, crude) as a yellow solid.

### Step 4: Synthesis of N-((4-methoxy-6-methyl-2-oxo-1,2-dihydropyridin-3-yl)methyl)-1-((2-(((4-methoxybenzyl)oxy)methyl)-1-methylpiperidin-4-yl)methyl)-2-methyl-1H-indole-3-carboxamide

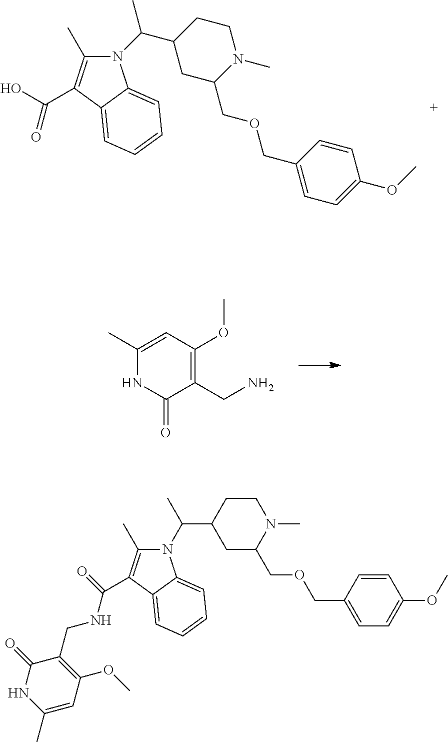

To a mixture of 1-[-1-[2-[(4-methoxyphenyl)methoxymethyl]-1-methyl-4-piperidyl]ethyl]-2-methyl-indole-3-carboxylic acid (310.00 mg, 0.688 mmol) and 3-(aminomethyl)-4-methoxy-6-methyl-1H-pyridin-2-one (173.58 mg, 1.03 mmol) in dichloromethane (10 mL) was added 2-(3H-[1,2,3]triazolo[4,5-b]pyridine-3-yl)-1,1,3,3-tetramethylisouronium hexafluorophosphate (523.21 mg, 1.38 mmol) and N-ethyl-N-isopropylpropan-2-amine (444.60 mg, 3.44 mmol) in one portion under N₂. The mixture was stirred for 4 hr. LCMS showed the reaction was complete. The mixture was poured into water (50 mL). The aqueous phase was extracted with DCM (50 mL*2). The combined organics phase was washed with saturated brine (100 mL), dried over anhydrous Na₂SO₄, filtered and concentrated. The residue was purified by silica gel chromatography (DCM/MeOH=40/1-10/1) to afford N-((4-methoxy-6-methyl-2-oxo-1,2-dihydropyridin-3-yl)methyl)-1-(1-(2-(((4-methoxybenzyl)oxy)methyl)-1-methylpiperidin-4-yl)ethyl)-2-methyl-1H-indole-3-carboxamide (220.00 mg, 366.21 umol, 53.23% yield) as a pale yellow solid.

### Step 5: Synthesis of 1-(1-(2-(hydroxymethyl)-1-methylpiperidin-4-yl)ethyl)-N-((4-methoxy-6-methyl-2-oxo-1,2-dihydropyridin-3-yl)methyl)-2-methyl-1H-indole-3-carboxamide

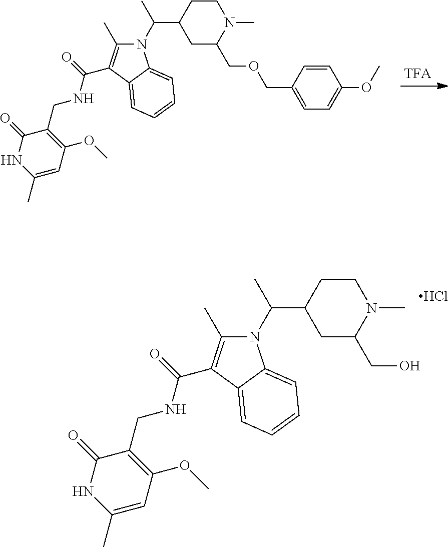 [SMILES: COc1ccc(COCC2CC(C(C)n3c(C)c(C(=O)NCc4c(OC)cc(C)[nH]c4=O)c4ccccc43)CCN2C)cc1 ; COc1cc(C)[nH]c(=O)c1CNC(=O)c1c(C)n(C(C)C2CCN(C)C(CO)C2)c2ccccc12 ; Cl]

To a mixture of N-((4-methoxy-6-methyl-2-oxo-1,2-dihydropyridin-3-yl)methyl)-1-(1-(2-(((4-methoxybenzyl)oxy)methyl)-1-methylpiperidin-4-yl)ethyl)-2-methyl-1H-indole-3-carboxamide (220.00 mg, 366.21 umol) in dichloromethane (2 mL) was added trifluoroacetic acid (835.11 mg, 7.32 mmol) in one portion under N₂. The mixture was stirred for 16 hr. LCMS showed the reaction was complete. The mixture was concentrated under reduced pressure. The residue was purified by silica gel chromatography to afford 1-(1-(2-(hydroxymethyl)-1-methylpiperidin-4-yl)ethyl)-N-((4-methoxy-6-methyl-2-oxo-1,2-dihydropyridin-3-yl)methyl)-2-methyl-1H-indole-3-carboxamide (100.00 mg, 0.21 mmol, 34.1% yield, 60% purity) as a yellow solid. This solid was purified by prep-HPLC (Instrument: Gilson 281, Column: Gemini C18 150*25 mm*10 um. Mobile phase A: water with 0.375% (v/v) HCl. Mobile phase B: MeCN, Column temperature: 40° C. Gradient: 25-55%-16 min. Flow rate: 22 ml/min) to afford 1-(1-(2-(hydroxymethyl)-1-methylpiperidin-4-yl)ethyl)-N-((4-methoxy-6-methyl-2-oxo-1,2-dihydropyridin-3-yl)methyl)-2-methyl-1H-indole-3-carboxamide hydrochloride (9.5 mg, 1.84 umol, 19% yield) as a white solid. LCMS m/z 481 [M+H]⁺. ¹H NMR (400 MHz, CD₃OD) δ 7.76 (d, J=8.4 Hz, 1H), 7.68 (d, J=7.2 Hz, 1H), 7.21-7.16 (m, 2H), 6.94 (s, 1H), 4.59 (s, 2H), 4.35-4.30 (m, 1H), 4.12 (s, 3H), 3.69-3.63 (m, 2H), 3.25-3.20 (m, 2H), 2.86-2.80 (m, 4H), 2.66 (s, 3H), 2.54 (s, 3H), 2.27-2.23 (m, 1H), 1.87-1.77 (m, 1H), 1.69-1.64 (m, 3H), 1.38-1.34 (m, 2H), 1.05-1.02 (m, 1H).

### Example 3: Synthesis of 1-(1-(2-(fluoromethyl)-1-methylpiperidin-4-yl)ethyl)-N-((4-methoxy-6-methyl-2-oxo-1,2-dihydropyridin-3-yl)methyl)-2-methyl-1H-indole-3-carboxamide Hydrochloride (29)

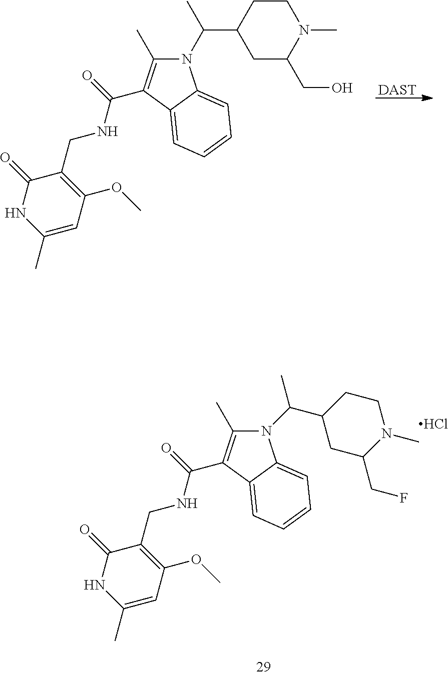

To a mixture of 1-[1-[2-(hydroxymethyl)-1-methyl-4-piperidyl]ethyl]-N-[(4-methoxy-6-methyl-2-oxo-1H-pyridin-3-yl)methyl]-2-methyl-indole-3-carboxamide (50.00 mg, 104 umol) in dichloromethane (3 mL) was added DAST (167.5 mg, 1.04 mmol) in portions at −78° C. under N₂. The mixture was stirred at −78° C. for 1 hr and 20° C. for 2 hr. LCMS showed the reaction was complete. The mixture was quenched by addition of saturated NaHCO₃ solution. The aqueous phase was extracted with DCM (40 mL*2). The combined organics phase was washed with saturated brine (50 mL), dried over anhydrous Na₂SO₄, filtered and concentrated. The crude residue was purified by prep-HPLC (Instrument: Gilson 281, Column: Gemini C18 150*25 mm*5 um. Mobile phase A: water with 0.375% (v/v) HCl. Mobile phase B: MeCN, Column temperature: 40° C. Gradient: 25-55%-16 min. Flow rate: 22 ml/min) to afford 1-(1-(2-(fluoromethyl)-1-methylpiperidin-4-yl)ethyl)-N-((4-methoxy-6-methyl-2-oxo-1,2-dihydropyridin-3-yl)methyl)-2-methyl-1H-indole-3-carboxamide hydrochloride (2.00 mg, 3.85 umol, 3.7% yield) as a white solid. LCMS m/z 483 [M+H]⁺. ¹H NMR (400 MHz, CD₃OD) δ 7.76-7.63 (m, 2H), 7.16 (d, J=8.8 Hz, 2H), 6.42 (s, 1H), 5.38-5.35 (m, 2H), 4.56 (s, 2H), 4.32 (s, 1H), 4.00 (s, 3H), 3.66 (s, 1H), 2.96 (s, 2H), 2.65 (s, 3H), 2.39 (s, 3H), 2.23-2.19 (m, 2H), 2.05-2.04 (m, 3H), 1.72-1.62 (m, 4H), 0.92-0.90 (m, 2H).

### Example 4: Synthesis of N-((4-methoxy-6-methyl-2-oxo-1,2-dihydropyridin-3-yl)methyl)-2-methyl-1-((1R)-1-(1,2,3,4-tetrahydroquinolin-4-yl)ethyl)-1H-indole-3-carboxamide Isomer A (1) and Isomer B (2)

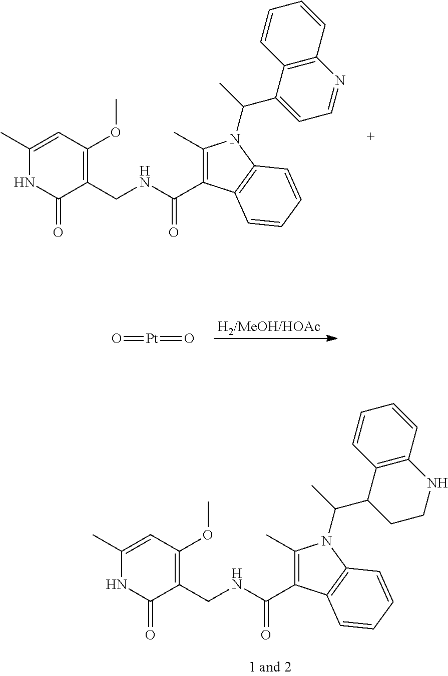

To a solution of N-((4-methoxy-6-methyl-2-oxo-1,2-dihydropyridin-3-yl)methyl)-2-methyl-1-(1-(quinolin-4-yl)ethyl)-1H-indole-3-carboxamide (200 mg, 416.18 umol) dissolved in acetic acid (1 mL) and methanol (8 mL) was added platinum(IV) oxide (220 mg, 968.82 umol). The suspension was degassed under vacuum and purged with hydrogen several times. The mixture was stirred under an atmosphere of hydrogen gas (1 atm) at room temperature for 10 hours. The reaction mixture was filtered and the filtrate was concentrated. The crude residue was purified by preparative HPLC (Mobile phase A: water with 0.05% aqueous ammonia solution; Mobile phase B: MeCN; column temperature: 30° C., Gradient: 41-42% B 12 min) to afford two products.

Isomer A: N-((4-methoxy-6-methyl-2-oxo-1,2-dihydropyridin-3-yl)methyl)-2-methyl-1-(1-(1,2,3,4-tetrahydroquinolin-4-yl)ethyl)-1H-indole-3-carboxamide (5.79 mg) as white solid. LCMS m/z 485 [M+H]⁺. ¹H NMR (400 MHz, METHANOL-d₄) δ 8.38 (d, J=5.6 Hz, 1H), 7.78-7.66 (m, 2H), 7.57 (d, J=5.2 Hz, 1H), 7.09-7.04 (m, 2H), 6.98 (t, J=8.0 Hz, 1H), 6.27 (s, 1H), 5.96-5.91 (m, 1H), 4.53 (d, J=4.0 Hz, 2H), 3.94 (s, 3H), 2.86-2.83 (m, 2H), 2.64-2.59 (m, 1H), 2.55 (s, 3H), 2.32 (s, 3H), 1.88 (d, J=6.8 Hz, 3H), 1.81-1.79 (m, 1H) 1.60-1.52 (m, 2H).

Isomer B: N-((4-methoxy-6-methyl-2-oxo-1,2-dihydropyridin-3-yl)methyl)-2-methyl-1-(1-(1,2,3,4-tetrahydroquinolin-4-yl)ethyl)-1H-indole-3-carboxamide (4.61 mg) as white solid. LCMS m/z 485 [M+H]⁺. ¹H NMR (400 MHz, METHANOL-d₄) δ 7.83-7.70 (m, 2H), 7.20-7.12 (m, 3H), 7.02-6.98 (m, 1H), 6.58-6.55 (m, 2H), 6.29 (s, 1H), 4.60-4.46 (m, 3H), 3.95 (s, 3H), 3.85 (d, J=11.6 Hz, 1H), 3.17-3.10 (m, 2H), 2.88 (s, 1H), 2.62 (s, 2H), 2.33 (s, 3H), 1.58 (d, J=7.2 Hz, 3H), 1.57-1.41 (m, 1H), 1.28-1.24 (m, 1H).

### Example 5: Synthesis of N-((4-methoxy-6-methyl-2-oxo-1,2-dihydropyridin-3-yl)methyl)-2-methyl-1-(1-(1-methyl-1,2,3,4-tetrahydroquinolin-4-yl)ethyl)-1H-indole-3-carboxamide (3)

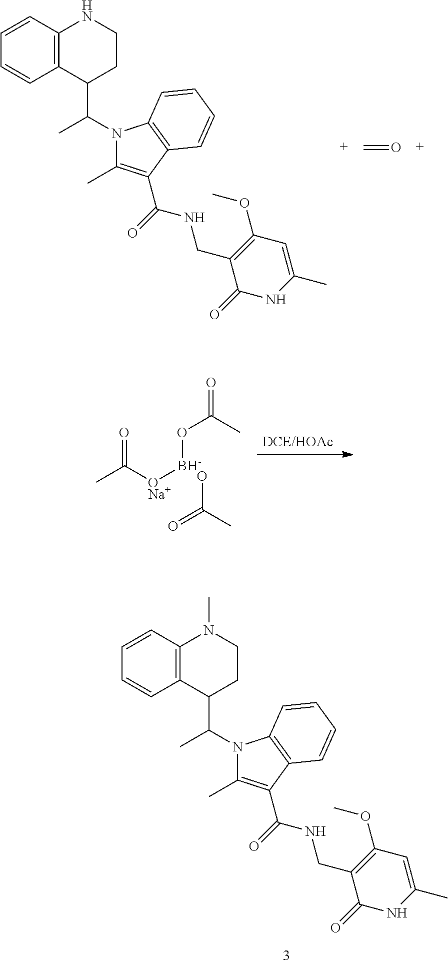

To a stirred solution of N-((4-methoxy-6-methyl-2-oxo-1,2-dihydropyridin-3-yl)methyl)-2-methyl-1-(1-(1,2,3,4-tetrahydroquinolin-4-yl)ethyl)-1H-indole-3-carboxamide (100 mg, 206 umol) in anhydrous dichloroethane (3 mL) and acetic acid (0.2 mL) was added an aqueous solution of formaldehyde (1 mL, 37% wt %, 4.85 mmol). The mixture was stirred for 2 hr at 50° C. Then the reaction solution was cooled to room temperature and sodium triacetoxyborohydride (411.12 mg, 1.94 mmol) was added. The mixture was then stirred for 16 hr at 50° C. The reaction was cooled to room temperature and the solution was quenched by addition of water (5 mL) and then concentrated. The aqueous solution was adjusted to pH=8 by addition of saturated sodium bicarbonate solution and then extracted with dichloromethane (10 mL×3). The combined organics layer was dried over anhydrous sodium sulfate, filtered, and concentrated. The crude residue was purified by preparative HPLC (Mobile phase A: water with 0.05% aqueous ammonia solution; Mobile phase B: MeCN; column temperature: 30° C., Gradient: 43-63% B 12 min) to afford N-((4-methoxy-6-methyl-2-oxo-1,2-dihydropyridin-3-yl)methyl)-2-methyl-1-(1-(1-methyl-1,2,3,4-tetrahydroquinolin-4-yl)ethyl)-1H-indole-3-carboxamide (3.99 mg, 8.00 umol, 8.25% yield) as white solid. LCMS m/z 499 [M+H]⁺.

¹H NMR (400 MHz, METHANOL-d₄) δ 7.80-7.71 (m, 2H), 7.22-7.11 (m, 4H), 6.71-6.66 (m, 2H), 6.29 (s, 1H), 4.60-4.50 (m, 3H), 3.95 (s, 3H), 3.83 (d, J=10.0 Hz, 1H), 3.17-3.12 (m, 1H), 2.97-2.92 (m, 1H), 2.90-2.86 (m, 4H), 2.62 (s, 2H), 2.33 (s, 3H), 1.63-1.48 (m, 4H), 1.35-1.32 (m, 1H).

### Example 6: Synthesis of 1-(1-(decahydroquinolin-4-yl)ethyl)-N-((4-methoxy-6-methyl-2-oxo-1,2-dihydropyridin-3-yl)methyl)-2-methyl-1H-indole-3-carboxamide (4)

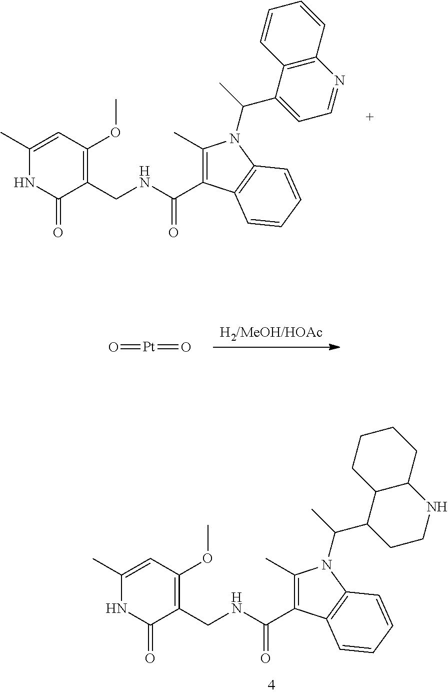

To a solution of N-((4-methoxy-6-methyl-2-oxo-1,2-dihydropyridin-3-yl)methyl)-2-methyl-1-(1-(1,2,3,4-tetrahydroquinolin-4-yl)ethyl)-1H-indole-3-carboxamide (100.00 mg, 208.09 umol) in acetic acid (0.5 mL) and methanol (4 mL) was added platinum(IV) oxide (236.27 mg, 1.04 mmol). The suspension was degassed under vacuum and purged with hydrogen three times. The mixture was stirred under hydrogen (15 psi) at room temperature for 12 hr. The reaction mixture was filtered and the filtrate was concentrated. The crude product was purified by preparative HPLC (Mobile phase A: water with 0.05% aqueous ammonia solution; Mobile phase B: MeCN; column temperature: 30° C., Gradient: 35-45% B 12 min) to give 1-(1-(decahydroquinolin-4-yl)ethyl)-N-((4-methoxy-6-methyl-2-oxo-1,2-dihydropyridin-3-yl)methyl)-2-methyl-1H-indole-3-carboxamide (2.83 mg, 2.77% yield) as white solid. LCMS m/z 491 [M+H]⁺. ¹H NMR (400 MHz, METHANOL-d₄) δ 7.71 (d, J=7.2 Hz, 1H), 7.61 (d, J=7.6 Hz, 1H), 7.14-7.07 (m, 2H), 6.28 (s, 1H), 4.53 (s, 2H), 4.42-4.34 (m, 1H), 3.95 (s, 3H), 2.98 (d, J=13.2 Hz, 2H), 2.59 (s, 3H), 2.45-2.44 (m, 1H), 2.33 (s, 3H), 2.05 (m, 1H), 1.94-1.74 (m, 4H), 1.64-1.59 (m, 5H), 1.31-1.26 (m, 2H), 0.52 (d, J=13.2 Hz, 1H).

### Example 7: Synthesis of 1-((1R)-1-(4-1-(2,2-difluoroethyl)-2-methylpiperidin-4-yl)ethyl)-N-((4-methoxy-6-methyl-2-oxo-1,2-dihydropyridin-3-yl)methyl)-2-methyl-1H-indole-3-carboxamide Isomer A (40) and Isomer B (41)

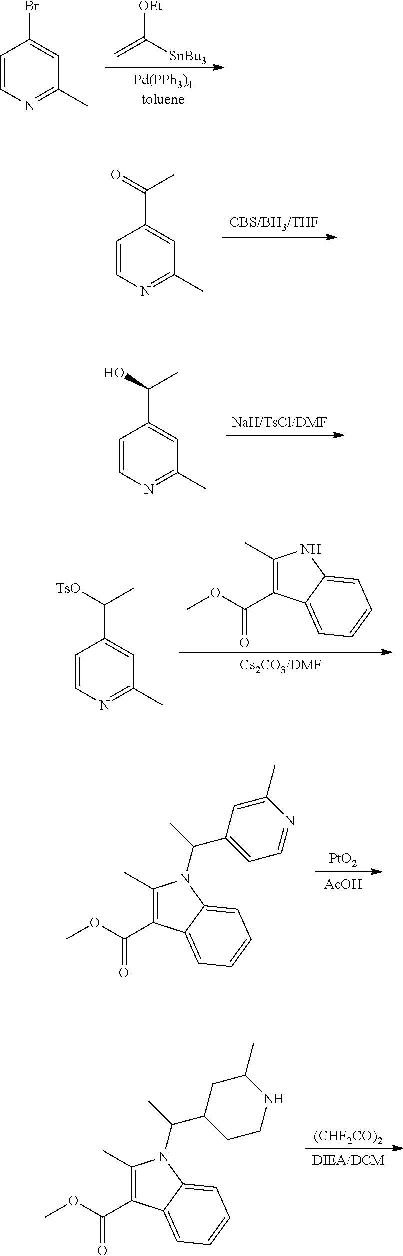

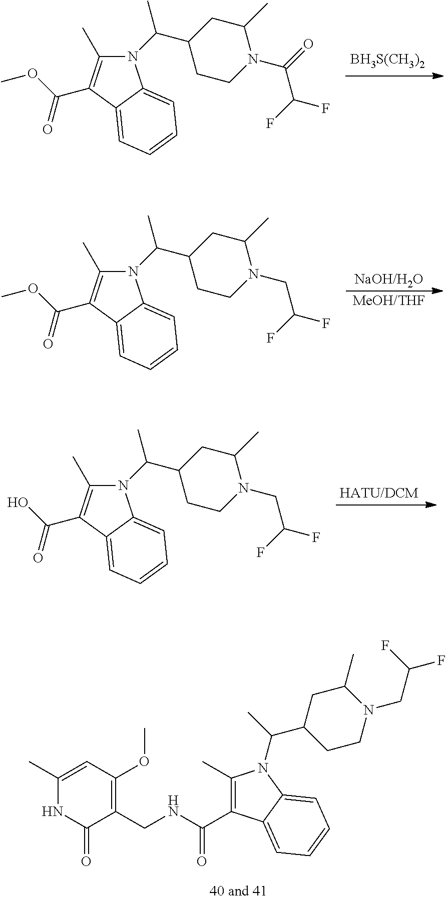

### Step 1: Synthesis of 1-(2-methylpyridin-4-yl)ethanone

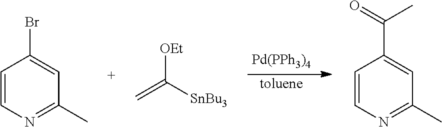

To a solution of 4-bromo-2-methylpyridine (10.0 g, 58 mmol) in toluene (100 mL) was added tributyl(1-ethoxyvinyl)stannane (39.7 g, 110 mmol) and Pd(PPh₃)₄ (5.8 g, 5 mmol). The mixture was stirred at 80° C. under a nitrogen atmosphere overnight. 1M HCl was added at 0° C. until the pH of the mixture was adjusted to 1-2. The mixture was stirred for 0.5 h. The pH was adjusted to 6-7 by adding 1M NaOH. The mixture was extracted with ethyl acetate (150 mL*3). The organics layer was washed with brine and dried over sodium sulfate. Concentration and purification by column chromatography on silica gel (petroleum ether/ethyl acetate=30/1-4/1) afforded 1-(2-methylpyridin-4-yl)ethanone as light yellow solid (3.0 g, 44%).

### Step 2: Synthesis of 1-(2-methylpyridin-4-yl)ethanol

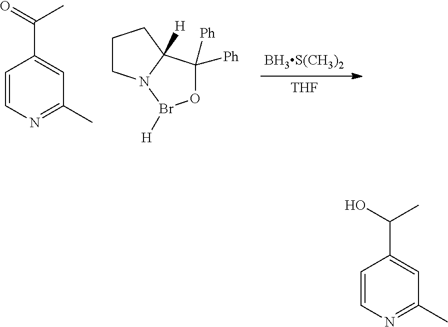

To a solution of (R)-(+)-2-Methyl-CBS-oxazaborolidine (609 mg, 2.2 mmol) in THF (30 mL) was added BH₃.S(CH₃)₂ (3.3 mL, 33 mmol), The mixture was stirred at room temperature for 1 h. 1-(2-methylpyridin-4-yl)ethanone (3.0 g, 22 mmol) was added at room temperature over 3h. The mixture was stirred at room temperature for 1 h before addition of MeOH (5 mL). The mixture was stirred at 80° C. overnight before the solution was concentrated under reduced pressure. Water (50 mL) was added and the mixture was extracted by ethyl acetate (100 mL*3). The organics layer was washed with brine, dried over sodium sulfate, and concentrated. The crude residue was purified by column chromatography on silica gel (petroleum ether/ethyl acetate=30/1-3/1) to afford 1-(2-methylpyridin-4-yl)ethanol as gray solid (2.4 g, 80%).

### Step 3: Synthesis of 1-(2-methylpyridin-4-yl)ethyl 4-methylbenzenesulfonate

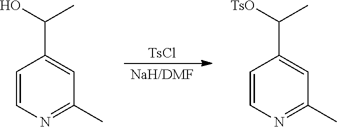

To a solution of 1-(2-methylpyridin-4-yl)ethanol (1.0 g, 7.1 mmol) in DMF (10 mL) was added sodium hydride (440 mg, 11 mmol, 60% in purity). The mixture was stirred at 0° C. for 0.5h. 4-methylbenzene-1-sulfonyl chloride (1.8 g, 9.3 mmol) was added and the mixture was stirred at room temperature overnight. Water (20 mL) was added and the solution was extracted with ethyl acetate (80 mL*3). The organics layer was washed with brine, dried over sodium sulfate, and concentrated. The crude residue was purified by column chromatography on silica gel (petroleum ether/ethyl acetate=30/1-2/1) to afford 1-(2-methylpyridin-4-yl)ethyl 4-methylbenzenesulfonate as brown oil (300 mg, 14%).

### Step 4: Synthesis of methyl 2-methyl-1-(1-(2-methylpyridin-4-yl)ethyl)-1H-indole-3-carboxylate

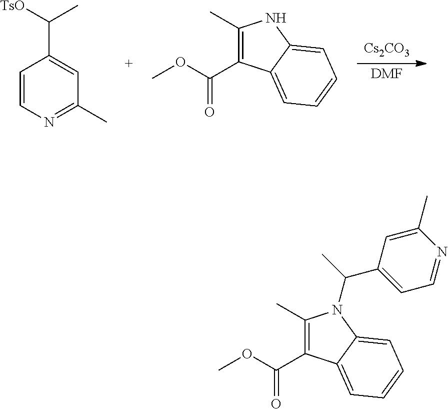

To a solution of 1-(2-methylpyridin-4-yl)ethyl 4-methylbenzenesulfonate (186 mg, 0.64 mmol) in DMF (5 mL) was added Cs₂CO₃ (417 mg, 1.28 mmol) and methyl 2-methyl-1H-indole-3-carboxylate (120 mg, 0.64 mmol). The mixture was stirred at 80° C. overnight. Water (10 mL) was added and the mixture was extracted with ethyl acetate (80 mL*3). The organics layer was washed with brine, dried over sodium sulfate, and concentrated. The crude residue was purified by column chromatography on silica gel (petroleum ether/ethyl acetate=30/1-2/1) to afford methyl 2-methyl-1-(1-(2-methylpyridin-4-yl)ethyl)-1H-indole-3-carboxylate as yellow oil (100 mg, 50%).

### Step 5: Synthesis of methyl 2-methyl-1-(1-(2-methylpiperidin-4-yl)ethyl)-1H-indole-3-carboxylate

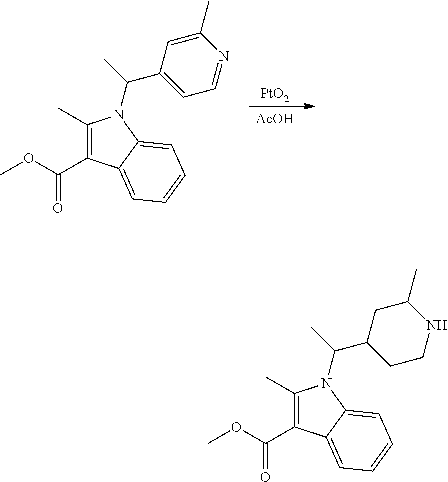

To a solution of methyl 2-methyl-1-(1-(2-methylpyridin-4-yl)ethyl)-1H-indole-3-carboxylate (100 mg, 0.33 mmol) in acetic acid (5 mL) was added palladium (IV) oxide (50 mg, 0.15 mmol). The mixture was stirred at 40° C. under an atmosphere of hydrogen (50 psi) overnight. The mixture was filtered and the filter cake washed with methanol (10 mL*3). The organics layer was concentrated to afford methyl 2-methyl-1-(1-(2-methylpiperidin-4-yl)ethyl)-1H-indole-3-carboxylate (100 mg, crude) which was used for subsequent steps without further purification.

### Step 6: Synthesis of methyl 1-(1-(1-(2,2-difluoroacetyl)-2-methylpiperidin-4-yl)ethyl)-2-methyl-1H-indole-3-carboxylate

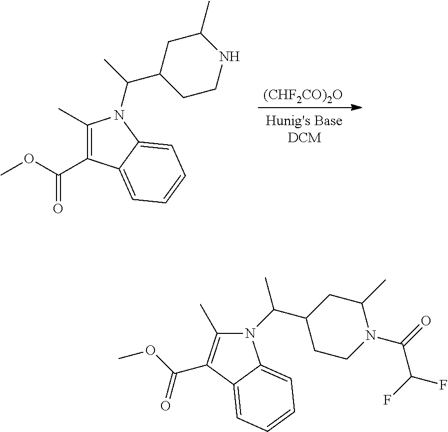

To a solution of methyl 2-methyl-1-(1-(2-methylpiperidin-4-yl)ethyl)-1H-indole-3-carboxylate (94 mg, 0.30 mmol) in DCM (5 mL) was added Hunig's base (78 mg, 0.60 mmol) and 2,2-difluoroacetic anhydride (87 mg, 0.50 mmol) at 0° C. The mixture was stirred at room temperature overnight. Water (10 mL) was added and the mixture was extracted with DCM (50 mL*3). The organics layer was washed with brine, dried over sodium sulfate, and concentrated. The crude residue was and purified by column chromatography on silica gel (petroleum ether/ethyl acetate=30/1-2/1) to afford methyl 1-(1-(1-(2,2-difluoroacetyl)-2-methylpiperidin-4-yl)ethyl)-2-methyl-1H-indole-3-carboxylate as yellow oil (100 mg, 85%).

### Step 7: Synthesis of methyl 1-(1-(1-(2,2-difluoroethyl)-2-methylpiperidin-4-yl)ethyl)-2-methyl-1H-indole-3-carboxylate

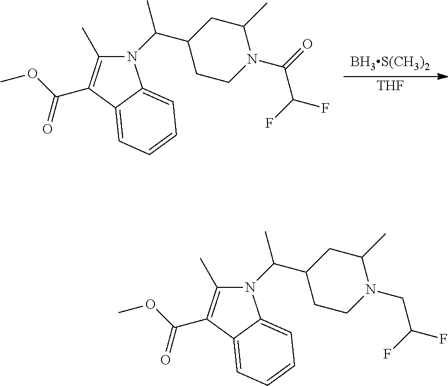 [SMILES: COC(=O)c1c(C)n(C(C)C2CCN(C(=O)C(F)F)C(C)C2)c2ccccc12 ; "BH3·S(CH3)2" ; COC(=O)c1c(C)n(C(C)C2CCN(CC(F)F)C(C)C2)c2ccccc12 ; "THF"]

To a solution of methyl 1-(1-(1-(2,2-difluoroethyl)-2-methylpiperidin-4-yl)ethyl)-2-methyl-1H-indole-3-carboxylate (100 mg, 0.25 mmol) in THF (5 mL) was added BH₃S(CH₃)₂ (0.1 mL, 1.0 mmol). The mixture was stirred at 80° C. for 3 hr before cooling to room temperature. Water (3 mL) was added and the mixture was concentrated to afford crude methyl 1-(1-(1-(2,2-difluoroethyl)-2-methylpiperidin-4-yl)ethyl)-2-methyl-1H-indole-3-carboxylate (60 mg, crude) for the next step.

### Step 8: Synthesis of 1-(1-(1-(2,2-difluoroethyl)-2-methylpiperidin-4-yl)ethyl)-2-methyl-1H-indole-3-carboxylic Acid

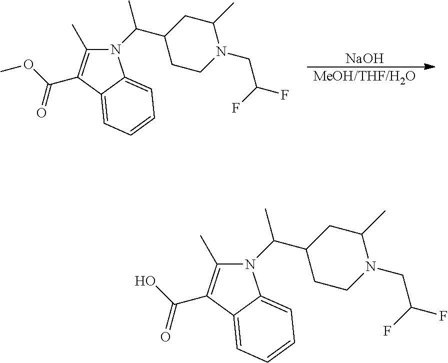 [SMILES: COC(=O)c1c(C)n(C(C)C2CCN(CC(F)F)C(C)C2)c2ccccc12 ; "NaOH" ; "MeOH/THF/H2O" ; Cc1c(C(=O)O)c2ccccc2n1C(C)C1CCN(CC(F)F)C(C)C1]

To a solution of methyl 1-(1-(1-(2,2-difluoroethyl)-2-methylpiperidin-4-yl)ethyl)-2-methyl-1H-indole-3-carboxylate (60 mg, 0.16 mmol) in MeOH/THF/H20(1:1:1, 6 mL) was added sodium hydroxide (630 mg, 1.6 mmol). The mixture was stirred at 80° C. for 40 hr. The solvent was removed under reduced pressure and the pH of the thick slurry was adjusted to 2-3 by addition of HCl (1M). The solution was concentrated to afford crude 1-(1-(1-(2,2-difluoroethyl)-2-methylpiperidin-4-yl)ethyl)-2-methyl-1H-indole-3-carboxylic acid (60 mg, crude) for the next step.

### Step 9: Synthesis of 1-(1-(1-(2,2-difluoroethyl)-2-methylpiperidin-4-yl)ethyl)-N-((4-methoxy-6-methyl-2-oxo-1,2-dihydropyridin-3-yl)methyl)-2-methyl-1H-indole-3-carboxamide

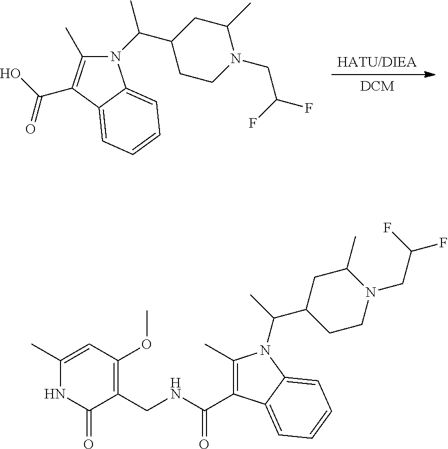 [SMILES: Cc1c(C(=O)O)c2ccccc2n1C(C)C1CCN(CC(F)F)C(C)C1 ; COc1cc(C)[nH]c(=O)c1CNC(=O)c1c(C)n(C(C)C2CCN(CC(F)F)C(C)C2)c2ccccc12]

To a solution of 1-(1-(1-(2,2-difluoroethyl)-2-methylpiperidin-4-yl)ethyl)-2-methyl-1H-indole-3-carboxylic acid (60 mg, 0.16 mmol) in dichloromethane (5 mL) was added HATU (91 mg, 0.24 mmol) and N-ethyl-N-isopropylpropan-2-amine (41 mg, 0.32 mmol). The mixture was stirred at room temperature for 0.5h. 3-(aminomethyl)-4-methoxy-6-methylpyridin-2(1H)-one hydrochloride (49 mg, 0.24 mmol) was added and the mixture was stirred at room temperature overnight. Water (10 mL) was added and the mixture was extracted with dichloromethane (30 mL*3). The organics layer was washed with brine, dried over sodium sulfate, and concentrated. The crude residue was purified by preparative HPLC (Condition: Column: ASB C18 150*25 mm; A: Water+0.1 mol/L NH₄HCO₃; B: CH₃CN; column temperature: 30° C.; Gradient: B in A 40-75%) to afford two isomers:

Isomer A: 1-(1-(1-(2,2-difluoroethyl)-2-methylpiperidin-4-yl)ethyl)-N-((4-methoxy-6-methyl-2-oxo-1,2-dihydropyridin-3-yl)methyl)-2-methyl-1H-indole-3-carboxamide (8 mg, 9%). LCMS m/z 515 [M+H]⁺. ¹H NMR (CD₃Cl₃, 400 MHz) δ 12.99 (s, 1H), 7.88 (d, J=7.6 Hz, 1H), 7.56 (s, 1H), 7.44 (d, J=8.0 Hz, 1H), 7.06 (m, 2H), 5.96-5.66 (m, 1H), 5.92 (s, 1H), 4.69 (dq, J=4.0 Hz, J=48.0 Hz, 2H), 4.13-4.04 (m, 1H), 3.91 (s, 3H), 3.12 (d, J=12.0 Hz, 1H), 2.09-3.94 (m, 1H), 2.71 (s, 3H), 2.67-2.55 (m, 1H), 2.44 (t, J=11.2 Hz, 1H), 2.37-2.25 (m, 1H), 2.19-2.09 (m, 4H), 1.94 (d, J=12.0 Hz, 1H), 1.58 (d, J=7.2 Hz, 3H), 1.44-1.31 (m, 1H), 0.98-0.91 (m, 1H), 0.88 (d, J=6.0 Hz, 3H). 0.85-0.76 (m, 1H).

Isomer B: 1-(1-(1-(2,2-difluoroethyl)-2-methylpiperidin-4-yl)ethyl)-N-((4-methoxy-6-methyl-2-oxo-1,2-dihydropyridin-3-yl)methyl)-2-methyl-1H-indole-3-carboxamide (15 mg, 16%) LCMS m/z 515 [M+H]⁺. ¹H NMR (CD₃Cl₃, 400 MHz) δ 12.84 (s., 1H), 7.87 (d, J=7.6 Hz, 1H), 7.54 (s, 1H), 7.44 (d, J=8.0 Hz, 1H), 7.11-7.00 (m, 2H), 5.92 (s, 1H), 5.92-5.64 (m, 1H), 4.67 (dq, J=8.0 Hz, J=32.0 Hz, 2H), 4.12-4.02 (m, 1H), 3.90 (s, 3H), 3.09-2.96 (m, 1H), 2.84-2.77 (m, 1H), 2.7 (s, 3H), 2.65-2.55 (m, 1H), 2.41 (m, 1H), 2.35-2.24 (m, 1H), 2.20-2.10 (m, 4H), 1.93 (d, J=12.0 Hz, 1H), 1.59 (d, J=7.2 Hz, 3H), 1.18 (d, J=6.0 Hz, 3H), 1.15-1.03 (m, 2H), 0.90 (d, J=12.4 Hz, 1H).

The following compounds were made following the procedures in Example 7, except using trifluoroacetic anhydride (TFAA) as the acylating reagent in step 6.

| Cmp. No. | Name | Structure | LCMS, ¹H NMR |
|---|---|---|---|
| 42 and 43 | N-((4-methoxy-6- methyl-2-oxo-1,2- dihydropyridin-3- yl)methyl)-2-methyl-1- (1-(2-methyl-1-(2,2,2- trifluoroethyl)piperidin- 4-yl)ethyl)-1H-indole-3- carboxamide Isomer A and Isomer B | 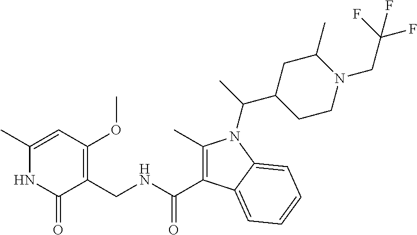 [SMILES: COc1cc(C)[nH]c(=O)c1CNC(=O)c1c(C)n(C(C)C2CCN(CC(F)(F)F)C(C)C2)c2ccccc12] | Isomer A: LCMS m/z 533 [M + H]⁺; ¹H NMR (CD₃Cl₃, 400 MHz) δ 12.56 (s, 1H), 7.88 (d, J = 7.6 Hz, 1H), 7.54 (s, 1H), 7.44 (d, J = 8.0 Hz, 1H), 7.13-7.00 (m, 2H), 5.92 (s, 1H), 4.68 (dq, J = 52.0 Hz, J = 6.0 Hz, 2H), 4.13- 4.03 (m, 1H), 3.91 (s, 3H), 3.26- 3.12 (m, 2H), 3.02-2.88 (m, 1H), 2.71 (s, 3H), 2.58 (t, J = 11.2 Hz, 1H), 2.38-2.23 (m, 2H), 2.19 (s, 3H), 1.94 (d, J = 12.4 Hz, 1H), 1.58 (d, J = 7.2 Hz, 3H), 1.43-1.33 (m, 1H), 1.00-0.73(m, 5H). |
| | | | Isomer B: LCMS m/z 533 |
| | | | [M + H]⁺; ¹H NMR (CD₃Cl₃, 400 |
| | | | MHz) δ 12.86 (s, 1H), 7.87 (d, |
| | | | J = 8.0 Hz, 1H), 7.58-7.51 (m, 1H), |
| | | | 7.44 (d, J = 8.0 Hz, 1H), 7.11-7.00 |
| | | | (m, 2H), 5.92 (s, 1H), 4.75-4.60 |
| | | | (m, 2H), 4.07 (dq, J = 5.6 Hz, |
| | | | J = 34.8 Hz, 1H), 3.90 (s, 3H), |
| | | | 3.25-3.12 (m, 1H), 3.02-2.89 (m, |
| | | | 1H), 2.83 (d, J = 7.6 Hz, 1H), 2.70 |
| | | | (s, 3H), 2.55 (s, 1H), 2.35-2.24 |
| | | | (m, 2H), 2.17 (s, 3H), 1.93 (d, |
| | | | J = 12.0 Hz, 1H), 1.59 (d, J = 7.2 |
| | | | Hz, 3H), 1.19 (d, J = 6.0 Hz, 3H), |
| | | | 1.13-1.02 (m, 2H), 0.86-0.93 (m, |
| | | | 1H). |

### Example 8: Synthesis of N-((4-methoxy-6-methyl-2-oxo-1,2-dihydropyridin-3-yl)methyl)-2-methyl-1-(1-(3-(trifluoromethyl)piperidin-4-yl)ethyl)-1H-indole-3-carboxamide (19)

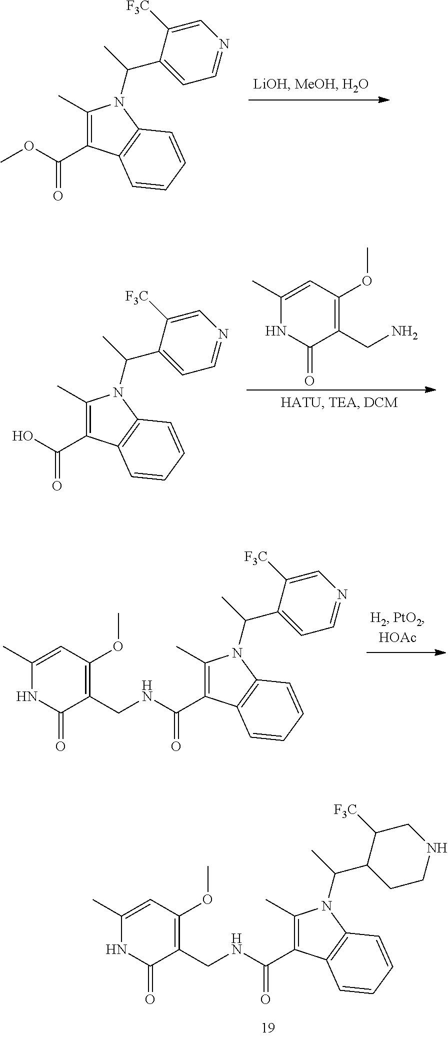 [SMILES: COC(=O)c1c(C)n(C(C)c2ccncc2C(F)(F)F)c2ccccc12 ; Cc1c(C(=O)O)c2ccccc2n1C(C)c1ccncc1C(F)(F)F ; COc1cc(C)[nH]c(=O)c1CN ; COc1cc(C)[nH]c(=O)c1CNC(=O)c1c(C)n(C(C)c2ccncc2C(F)(F)F)c2ccccc12 ; COc1cc(C)[nH]c(=O)c1CNC(=O)c1c(C)n(C(C)C2CCNCC2C(F)(F)F)c2ccccc12]

### Step 1: Synthesis of 2-methyl-1-(1-(3-(trifluoromethyl)pyridin-4-yl)ethyl)-1H-indole-3-carboxylic Acid

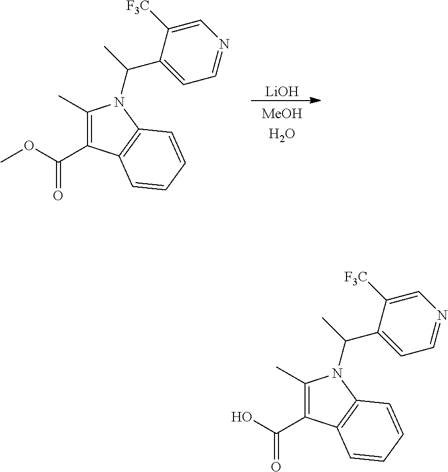 [SMILES: COC(=O)c1c(C)n(C(C)c2ccncc2C(F)(F)F)c2ccccc12 ; "LiOH" ; Cc1c(C(=O)O)c2ccccc2n1C(C)c1ccncc1C(F)(F)F]

To a solution of methyl 2-methyl-1-(1-(3-(trifluoromethyl)pyridin-4-yl)ethyl)-1H-indole-3-carboxylate (150 mg, 0.414 mmol) in methanol (3.0 mL), tetrahydrofuran (1.0 mL), and water (1.0 mL) was added lithium hydroxide hydrate (174 mg, 4.14 mmol) and stirred at 60° C. for 17 hr. The reaction solution was concentrated and adjusted pH to 5-6 with HCl solution (1 M) and then extracted with dichloromethane. The organic phase was washed with brine, dried over sodium sulfate and concentrated to afford 2-methyl-1-(1-(3-(trifluoromethyl)pyridin-4-yl)ethyl)-1H-indole-3-carboxylic acid (80 mg, 55.5% yield). LCMS m/z: 349 [M+H]⁺.

### Step 2: Synthesis of N-((4-methoxy-6-methyl-2-oxo-1,2-dihydropyridin-3-yl)methyl)-2-methyl-1-(1-(3-(trifluoromethyl)pyridin-4-yl)ethyl)-1H-indole-3-carboxamide

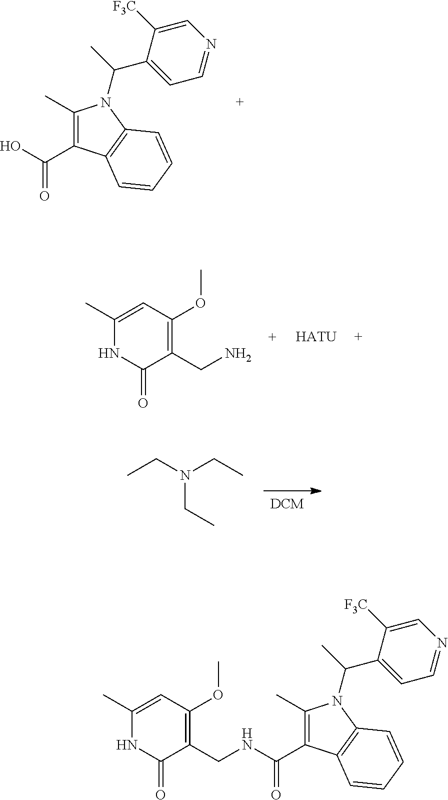

To a solution of 2-methyl-1-(1-(3-(trifluoromethyl)pyridin-4-yl)ethyl)-1H-indole-3-carboxylic acid (80 mg, 0.23 mmol) in dichloromethane (2 mL) was added 3-(aminomethyl)-4-methoxy-6-methylpyridin-2(1H)-one (46.4 mg, 0.27 mmol), 2-(3H-[1,2,3]triazolo[4,5-b]pyridin-3-yl)-1,1,3,3-tetramethylisouroniumhexafluorophosphate(V) (102.6 mg, 0.27 mmol), triethylamine (69.7 mg, 0.69 mmol) and the reaction was stirred at room temperature for 17 hr. The reaction was diluted with water and extracted with dichloromethane. The organic phase was dried over sodium sulfate and purified by prep-TLC (dichloromethane:methanol=10:1) to afford N-((4-methoxy-6-methyl-2-oxo-1,2-dihydropyridin-3-yl)methyl)-2-methyl-1-(1-(3-(trifluoromethyl)pyridin-4-yl)ethyl)-1H-indole-3-carboxamide (50 mg, 43.7% yield) as a yellow oil. LCMS m/z 499.1 [M+H]⁺.

### Step 3: Synthesis of N-((4-methoxy-6-methyl-2-oxo-1,2-dihydropyridin-3-yl)methyl)-2-methyl-1-(1-(3-(trifluoromethyl)piperidin-4-yl)ethyl)-1H-indole-3-carboxamide

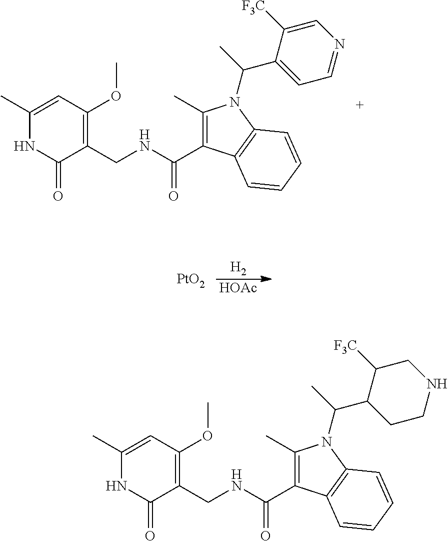 [SMILES: COc1cc(C)[nH]c(=O)c1CNC(=O)c1c(C)n(C(C)c2ccncc2C(F)(F)F)c2ccccc12 ; "H2" ; "PtO2" ; COc1cc(C)[nH]c(=O)c1CNC(=O)c1c(C)n(C(C)C2CCNCC2C(F)(F)F)c2ccccc12 ; "HOAc"]

To a solution of N-((4-methoxy-6-methyl-2-oxo-1,2-dihydropyridin-3-yl)methyl)-2-methyl-1-(1-(3-(trifluoromethyl)pyridin-4-yl)ethyl)-1H-indole-3-carboxamide (50 mg, 0.1 mmol) in acetic acid (2 mL) was added PtO₂ (11.4 mg, 0.05 mmol) and the reaction stirred at room temperature under a hydrogen balloon for 17 hr. The solution was then heated to 50° C. and stirred for 4.5 hours. The mixture was filtered and the filtrate was diluted with between water and extracted with ethyl acetate. The organic phase was dried over sodium sulfate, concentrated and purified by preparative-HPLC (Condition: Column: ASB C18 150*25 mm; Mobile phase A: MeCN; Mobile phase B: water with 0.1% hydrochloride solution; column temperature: 30° C.; Gradient: B in A, 20-50%; Flow rate: 25 ml/min) to afford N-((4-methoxy-6-methyl-2-oxo-1,2-dihydropyridin-3-yl)methyl)-2-methyl-1-(1-(3-(trifluoromethyl)piperidin-4-yl)ethyl)-1H-indole-3-carboxamide (1.6 mg, 3.16% yield). LCMS m/z 505 [M+H]⁺. ¹H NMR (400 MHz, METHANOL-d₄) δ 7.80-7.70 (m, 2H), 7.24-7.15 (m, 2H), 6.97 (br. s., 1H), 4.59 (br. s., 2H), 4.13 (s, 3H), 3.84 (br. s., 2H), 3.64 (br. s., 1H), 3.45 (d, J=12.8 Hz, 2H), 3.19 (d, J=10.4 Hz, 1H), 2.81 (br. s., 1H), 2.68 (br. s., 1H), 2.66-2.61 (m, 2H), 2.55 (s, 3H), 1.88 (d, J=7.2 Hz, 1H), 1.78 (d, J=6.8 Hz, 2H), 1.57 (d, J=7.6 Hz, 1H), 1.28 (br. s., 1H)

### Example 9: Synthesis of N-((4-methoxy-6-methyl-2-oxo-1,2-dihydropyridin-3-yl)methyl)-2-methyl-1-(1-(1-(oxetan-3-yl)-3-(trifluoromethyl)piperidin-4-yl)ethyl)-1H-indole-3-carboxamide (8)

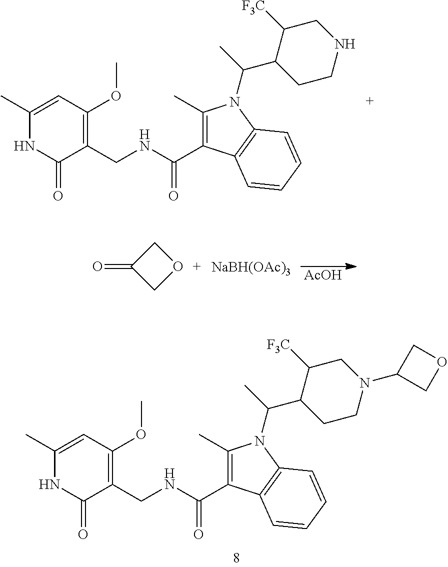 [SMILES: "AcOH" ; COc1cc(C)[nH]c(=O)c1CNC(=O)c1c(C)n(C(C)C2CCNCC2C(F)(F)F)c2ccccc12 ; O=C1COC1 ; COc1cc(C)[nH]c(=O)c1CNC(=O)c1c(C)n(C(C)C2CCN(C3COC3)CC2C(F)(F)F)c2ccccc12 ; "NaBH(OAc)3"]

To a solution of N-[(4-methoxy-6-methyl-2-oxo-1H-pyridin-3-yl)methyl]-2-methyl-1-[(1R)-1-[3-(trifluoromethyl)-4-piperidyl]ethyl]indole-3-carboxamide (40.00 mg, 79.28 umol, 1.00 Eq) and oxetan-3-one (28.56 mg, 396.40 umol, 5.00 Eq) in 1,2-dichloroethane (1.5 mL) was added AcOH (5.71 mg, 95.14 umol, 1.20 Eq) and the reaction was stirred at room temperature for 2 hrs. To this mixture was added NaBH(AcO)₃ (50.41 mg, 237.84 umol, 3.00 Eq) and reaction was stirred for an additional 20 hr. The reaction was diluted with water and extracted with DCM. The combined organics phase was washed with brine, dried over Na₂SO₄ and concentrated. The crude residue was purified by preparative-HPLC(A: Water+0.1% HCl; B: CH₃CN; Gradient: B in A 20-50%) to afford N-((4-methoxy-6-methyl-2-oxo-1,2-dihydropyridin-3-yl)methyl)-2-methyl-1-((1R)-1-(1-(oxetan-3-yl)-3-(trifluoromethyl)piperidin-4-yl)ethyl)-1H-indole-3-carboxamide (1.6 mg, 3.60%) as yellow solid. LCMS m/z 561 [M+H]⁺. ¹H NMR (400 MHz, METHANOL-d₄) δ 7.79-7.69 (m, 2H), 7.24-7.19 (m, 2H), 6.89 (br. s., 1H), 4.59 (s, 2H), 4.44-4.28 (m, 2H), 4.11 (s, 3H), 4.07-3.89 (m, 3H), 3.87-3.73 (m, 2H), 3.70-3.62 (m, 1H), 3.52-2.47 (m, 2H), 2.64 (s, 3H), 2.53 (s, 3H), 1.79 (d, J=6.4 Hz, 3H), 1.28 (br. s., 2H), 0.88 (d, J=9.2 Hz, 2H).

### Example 10: Synthesis of N-((4-methoxy-6-methyl-2-oxo-1,2-dihydropyridin-3-yl)methyl)-2-methyl-1-(1-(1-methyl-3-(trifluoromethyl)piperidin-4-yl)ethyl)-1H-indole-3-carboxamide Isomer A (27) and Isomer B (28)

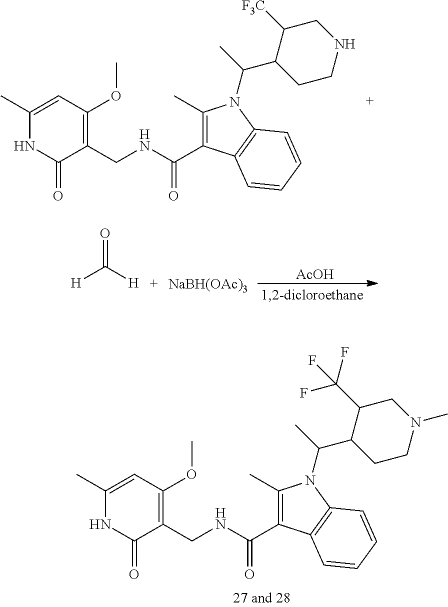

To a solution of N-[(4-methoxy-6-methyl-2-oxo-1H-pyridin-3-yl)methyl]-2-methyl-1-[(1R)-1-[3-(trifluoromethyl)-4-piperidyl]ethyl]indole-3-carboxamide (180.00 mg, 356.76 umol, 1.00 Eq) in 1,2-dichloroethane (3 mL) was added a solution of formaldehyde in water (289.75 mg, 3.57 mmol, 10.01 Eq) and AcOH (226.84 mg, 1.07 mmol, 3.00 Eq) and the reaction was stirred at 40° C. for 2 hr. To this solution was added NaBH(AcO)₃ (226.84 mg, 1.07 mmol, 3.00 Eq) and the mixture was stirred at 40° C. for 12 hr. The reaction was cooled to room temperature and diluted with water, extracted with DCM. The organic phase was washed with brine, dried over Na₂SO₄, and concentrated. The crude residue was purified by SFC to afford two isomers.

Isomer A: N-((4-methoxy-6-methyl-2-oxo-1,2-dihydropyridin-3-yl)methyl)-2-methyl-1-(1-(1-methyl-3-(trifluoromethyl)piperidin-4-yl)ethyl)-1H-indole-3-carboxamide (1.5 mg, Yield=0.81%) as solid. LCMS m/z 519 [M+H]⁺. ¹H NMR (400 MHz, CHLOROFORM-d) δ 12.01 (br. s., 1H), 7.81 (d, J=8.4 Hz, 1H), 7.43 (t, J=5.6 Hz, 1H), 7.33 (d, J=8.8 Hz, 1H), 7.05-6.97 (m, 2H), 5.85 (s, 1H), 4.68-4.47 (m, 3H), 3.83 (s, 3H), 3.17 (d, J=12.0 Hz, 1H), 2.71-2.52 (m, 5H), 2.49-2.37 (m, 1H), 2.12 (d, J=2.8 Hz, 6H), 1.97 (d, J=8.0 Hz, 1H), 1.64 (d, J=6.8 Hz, 3H), 0.87-0.73 (m, 3H).

Isomer B: N-((4-methoxy-6-methyl-2-oxo-1,2-dihydropyridin-3-yl)methyl)-2-methyl-1-(1-(1-methyl-3-(trifluoromethyl)piperidin-4-yl)ethyl)-1H-indole-3-carboxamide (1.4 mg, Yield=0.76%) as solid. LCMS m/z 519 [M+H]⁺. ¹H NMR (400 MHz, CHLOROFORM-d) δ 7.86 (d, J=6.4 Hz, 1H), 7.39 (d, J=8.0 Hz, 2H), 7.33 (d, J=7.6 Hz, 1H), 7.13-7.04 (m, 2H), 5.93 (s, 1H), 4.70-4.55 (m, 3H), 3.90 (s, 3H), 3.23 (d, J=12.4 Hz, 1H), 2.69 (s, 5H), 2.56-2.45 (m, 1H), 2.19 (d, J=9.2 Hz, 6H), 2.07-1.98 (m, 1H), 1.70 (d, J=6.8 Hz, 3H), 1.02-0.80 (m, 3H).

### Example 11: Synthesis of 1-(1-(2-(difluoromethyl)piperidin-4-yl)ethyl)-N-((4-methoxy-6-methyl-2-oxo-1,2-dihydropyridin-3-yl)methyl)-2-methyl-1H-indole-3-carboxamide Isomer A (36) and Isomer B (37)

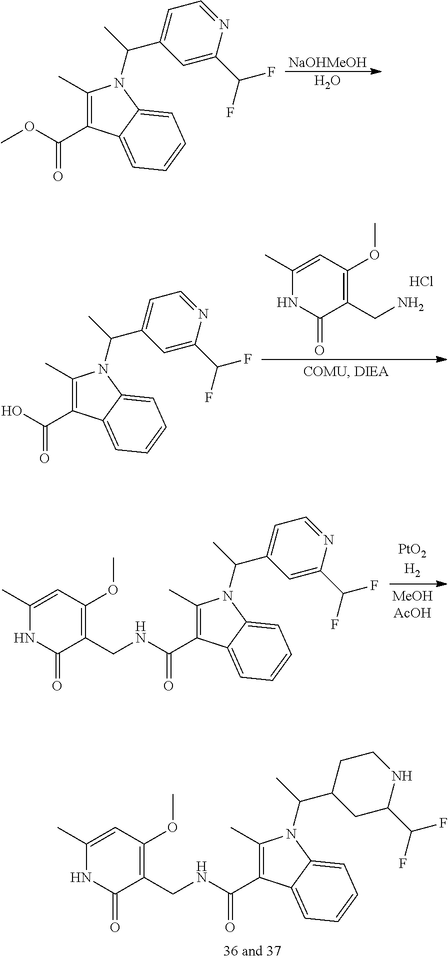

### Step 1: Synthesis of 1-(1-(2-(difluoromethyl)pyridin-4-yl)ethyl)-2-methyl-1H-indole-3-carboxylic Acid

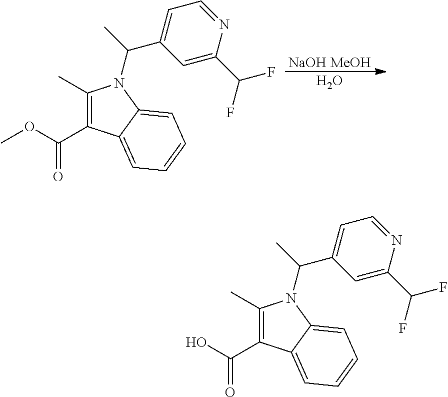

To a solution of methyl 1-(1-(2-(difluoromethyl)pyridin-4-yl)ethyl)-2-methyl-1H-indole-3-carboxylate (770.00 mg, 2.24 mmol) in methanol (45 mL) and water (15 mL) was added sodium hydroxide (358.40 mg, 8.96 mmol) in one portion at room temperature. The mixture was stirred at 120° C. overnight. The mixture was concentrated and the residue was acidified to pH=6 with hydrogen chloride solution (2M) at 0° C. The acidic solution was extracted with ethyl acetate (100 mL×3). The combined organics phase was dried over anhydrous Na₂SO₄, filtered, and concentrated to afford crude 1-(1-(2-(difluoromethyl)pyridin-4-yl)ethyl)-2-methyl-1H-indole-3-carboxylic acid.

### Step 2: Synthesis of 1-(1-(2-(difluoromethyl)pyridin-4-yl)ethyl)-N-((4-methoxy-6-methyl-2-oxo-1,2-dihydropyridin-3-yl)methyl)-2-methyl-1H-indole-3-carboxamide

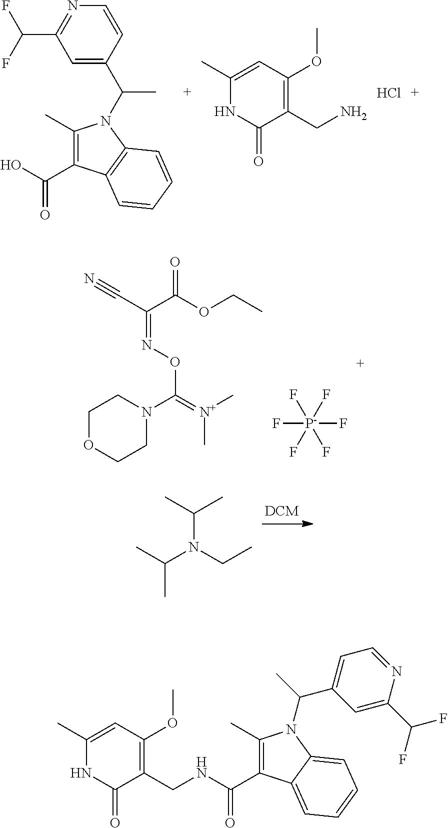

To a mixture of 1-(1-(2-(difluoromethyl)pyridin-4-yl)ethyl)-2-methyl-1H-indole-3-carboxylic acid (600.00 mg, 1.45 mmol) and 3-(aminomethyl)-4-methoxy-6-methylpyridin-2(1H)-one (370.93 mg, 1.81 mmol) in dichloromethane (30 mL) was added 1-cyano-2-ethoxy-2-oxoethylidenaminooxy)dimethylamino-morpholino-carbenium hexafluorophosphate (931.5 mg, 2.17 mmol) and N-ethyl-N-isopropylpropan-2-amine (562.2 mg, 4.35 mmol). The mixture was stirred at room temperature overnight. The reaction was quenched with water and extracted with ethyl acetate (200 mL×3). The combined organic phase was dried over anhydrous Na₂SO₄, filtered and concentrated. The crude residue was purified by perparative TLC (ethyl acetate:methanol=5:1) to afford 1-(1-(2-(difluoromethyl)pyridin-4-yl)ethyl)-N-((4-methoxy-6-methyl-2-oxo-1,2-dihydropyridin-3-yl)methyl)-2-methyl-1H-indole-3-carboxamide (400.00 mg, 0.83 mmol, yield 57.41%) as a yellow solid.

### Step 3: Synthesis of 1-(1-(2-(difluoromethyl)piperidin-4-yl)ethyl)-N-((4-methoxy-6-methyl-2-oxo-1,2-dihydropyridin-3-yl)methyl)-2-methyl-1H-indole-3-carboxamide

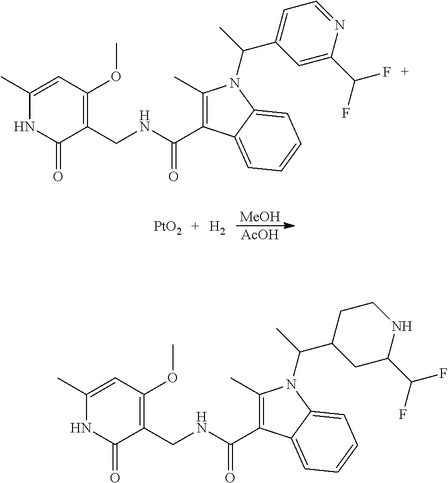 [SMILES: "MeOH" ; "AcOH" ; COc1cc(C)[nH]c(=O)c1CNC(=O)c1c(C)n(C(C)c2ccnc(C(F)F)c2)c2ccccc12 ; "H2" ; "PtO2" ; COc1cc(C)[nH]c(=O)c1CNC(=O)c1c(C)n(C(C)C2CCNC(C(F)F)C2)c2ccccc12]

To a mixture of 1-(1-(2-(difluoromethyl)pyridin-4-yl)ethyl)-N-((4-methoxy-6-methyl-2-oxo-1,2-dihydropyridin-3-yl)methyl)-2-methyl-1H-indole-3-carboxamide (100.00 mg, 0.2 mmol) in methanol (8 mL) and acetic acid (1 mL) was added platinum oxide (94.52 mg, 0.41 mmol). The mixture was stirred at room temperature for 3.5h under an atmosphere of H₂ (1 atm). The reaction was filtrated and the filtrate concentrated. The residue was basified to pH=8 with sodium hydrogencarbonate solution and extracted with dichloromethane (40 mL×6). The combined organics phase was dried over anhydrous Na₂SO₄, filtered and concentrated. The crude residue was purified by HPLC (Mobile phase A: water with 0.05% ammonia solution; Mobile phase B: MeCN; column temperature: 40° C., Gradient: 45-75% B 10 min) to afford two isomers.

Isomer A: 1-(1-(2-(difluoromethyl)piperidin-4-yl)ethyl)-N-((4-methoxy-6-methyl-2-oxo-1,2-dihydropyridin-3-yl)methyl)-2-methyl-1H-indole-3-carboxamide (2.70 mg, 5.55 umol, yield 2.67%). LCMS m/z 487 [M+H]⁺. ¹H NMR (400 MHz, METHANOL-d₄) δ 7.72 (d, J=7.03 Hz, 1H), 7.59 (d, J=8.03 Hz, 1H), 7.16-7.07 (m, 2H), 6.28 (s, 1H), 5.46 (d, J=3.51 Hz, 1H), 4.54 (s, 2H), 4.27-4.16 (m, 1H), 3.95 (s, 3H), 3.20 (d, J=12.05 Hz, 1H), 2.75-2.65 (m, 2H), 2.61 (s, 3H), 2.54-2.43 (m, 1H), 2.32 (s, 3H), 2.04 (d, J=13.05 Hz, 1H), 1.63 (d, J=6.53 Hz, 3H), 1.33-1.22 (m, 1H), 0.99 (d, J=12.05 Hz, 1H), 0.85 (q, J=12.05 Hz, 1H).

Isomer B: 1-(1-(2-(difluoromethyl)piperidin-4-yl)ethyl)-N-((4-methoxy-6-methyl-2-oxo-1,2-dihydropyridin-3-yl)methyl)-2-methyl-1H-indole-3-carboxamide (2.80 mg, 5.75 umol, yield 2.76%) as white solid. LCMS m/z 487 [M+H]⁺. ¹H NMR (400 MHz, METHANOL-d₄) δ 7.72 (d, J=7.53 Hz, 1H), 7.59 (d, J=7.53 Hz, 1H), 7.15-7.07 (m, 2H), 6.29 (s, 1H), 5.73 (d, J=4.02 Hz, 1H), 4.54 (s, 2H), 4.23 (dd, J=10.54, 7.03 Hz, 1H), 3.95 (s, 3H), 2.91 (d, J=12.55 Hz, 1H), 2.73 (d, J=4.52 Hz, 1H), 2.66-2.60 (m, 3H), 2.48 (d, J=11.04 Hz, 1H), 2.43-2.35 (m, 1H), 2.33 (s, 3H), 2.13 (d, J=12.55 Hz, 1H), 1.67-1.61 (m, 3H), 1.20-1.09 (m, 1H), 1.02-0.93 (m, 1H), 0.90-0.80 (m, 1H).

### Example 12: Synthesis of 1-(1-(2-(4-fluorophenyl)piperidin-4-yl)ethyl)-N-((4-methoxy-6-methyl-2-oxo-1,2-dihydropyridin-3-yl)methyl)-2-methyl-1H-indole-3-carboxamide Isomer A (31) and Isomer B (32)

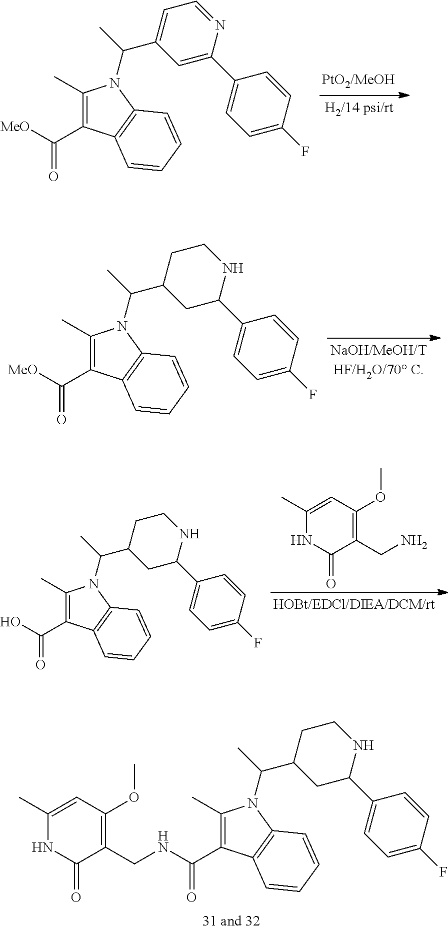 [SMILES: COC(=O)c1c(C)n(C(C)c2ccnc(-c3ccc(F)cc3)c2)c2ccccc12 ; COC(=O)c1c(C)n(C(C)C2CCNC(c3ccc(F)cc3)C2)c2ccccc12 ; Cc1c(C(=O)O)c2ccccc2n1C(C)C1CCNC(c2ccc(F)cc2)C1 ; COc1cc(C)[nH]c(=O)c1CN ; COc1cc(C)[nH]c(=O)c1CNC(=O)c1c(C)n(C(C)C2CCNC(c3ccc(F)cc3)C2)c2ccccc12]

### Step 1: Synthesis of methyl 1-(1-(2-(4-fluorophenyl)piperidin-4-yl)ethyl)-2-methyl-1H-indole-3-carboxylate

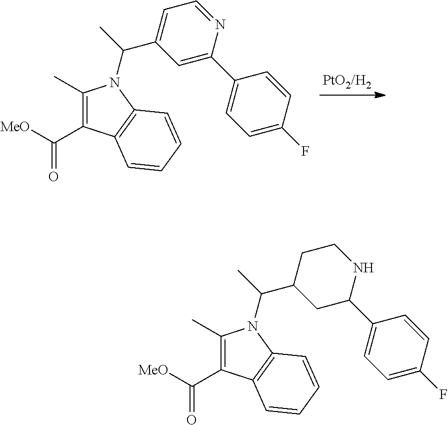 [SMILES: COC(=O)c1c(C)n(C(C)c2ccnc(-c3ccc(F)cc3)c2)c2ccccc12 ; COC(=O)c1c(C)n(C(C)C2CCNC(c3ccc(F)cc3)C2)c2ccccc12]

To a solution of methyl 1-(1-(2-(4-fluorophenyl)pyridin-4-yl)ethyl)-2-methyl-1H-indole-3-carboxylate (1 g, 2.58 mmol) in methanol (16 mL) and acetic acid (2 mL) was added platinum(IV) oxide (750 mg, 3.3 mmol) at room temperature. The solution was stirred for 22 hours under an H₂ (1 atm) atmosphere. The solution was filtered and the filtrate was concentrated to afford crude methyl 1-(1-(2-(4-fluorophenyl)piperidin-4-yl)ethyl)-2-methyl-1H-indole-3-carboxylate (700 mg, 68.78% yield) as yellow oil.

### Step 2: Synthesis of 1-(1-(2-(4-fluorophenyl)piperidin-4-yl)ethyl)-2-methyl-1H-indole-3-carboxylic Acid

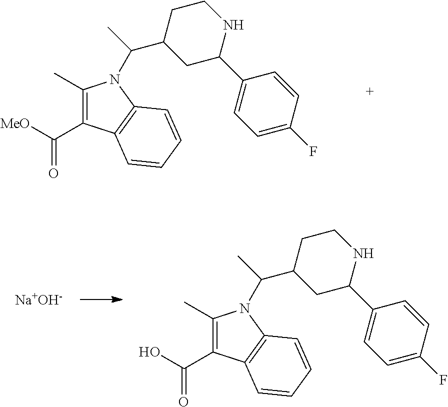 [SMILES: "Na+OH-" ; COC(=O)c1c(C)n(C(C)C2CCNC(c3ccc(F)cc3)C2)c2ccccc12 ; Cc1c(C(=O)O)c2ccccc2n1C(C)C1CCNC(c2ccc(F)cc2)C1]

To a solution of methyl 1-(1-(2-(4-fluorophenyl)piperidin-4-yl)ethyl)-2-methyl-1H-indole-3-carboxylate (700.00 mg, 1.77 mmol) in methanol (4 mL), water (2 mL), and tetrahydrofuran (4 mL) was added sodium hydroxide (354.00 mg, 8.85 mmol). The solution was stirred for 10 hr at 80° C. The solution was concentrated and the slurry was diluted with water. The resulting solution was acidified by addition of HCl (2M) to pH=6. The solution was extracted with ethyl acetate and the organics layer was concentrated to afford crude 1-(1-(2-(4-fluorophenyl)piperidin-4-yl)ethyl)-2-methyl-1H-indole-3-carboxylic acid (350.00 mg, 51.97% yield) as a brown solid.

### Step 3: Synthesis of 1-(1-(2-(4-fluorophenyl)piperidin-4-yl)ethyl)-N-((4-methoxy-6-methyl-2-oxo-1,2-dihydropyridin-3-yl)methyl)-2-methyl-1H-indole-3-carboxamide

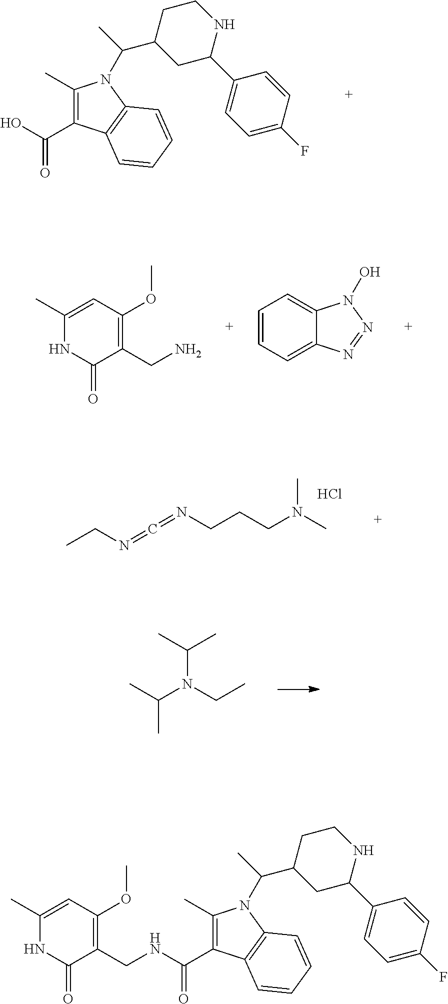

To a solution of 1-(1-(2-(4-fluorophenyl)piperidin-4-yl)ethyl)-2-methyl-1H-indole-3-carboxylic acid (120.00 mg, 315.42 umol) in dichloromethane (3 mL) was added 1H-benzo[d][1,2,3]triazol-1-ol (63.93 mg, 473.12 umol), N1-((ethylimino)methylene)-N3,N3-dimethylpropane-1,3-diamine hydrochloride (120.93 mg, 630.83 umol), N-ethyl-N-isopropylpropan-2-amine (122.29 mg, 946.25 umol) and 3-(aminomethyl)-4-methoxy-6-methylpyridin-2(1H)-one (106.10 mg, 630.83 umol). The reaction mixture was stirred at room temperature for 18 hr. The mixture was diluted with water and extracted with dichloromethane. The organic layer was concentrated and the crude residue was purified by preparative-HPLC (Instrument: Gilson GX281 Column: Grace C18 150*25 mm*5 um Mobile phase A: water with 0.1% hydrogen chloride solution Mobile phase B: acetonitrile column temperature: 40° C. Gradient 12-42% B 22 min) to afford two isomers:

Isomer A: 1-(1-(2-(4-fluorophenyl)piperidin-4-yl)ethyl)-N-((4-methoxy-6-methyl-2-oxo-1,2-dihydropyridin-3-yl)methyl)-2-methyl-1H-indole-3-carboxamide (6.00 mg, 11.31 umol, 3.58% yield). LCMS m/z 531 [M+H]⁺. ¹H NMR (400 MHz, Methanol-d₄) δ 7.83-7.70 (m, 2H), 7.66 (br. s., 2H), 7.29-7.13 (m, 4H), 6.91 (br. s., 1H), 4.61 (br. s., 1H), 4.44 (br. s., 2H), 4.13 (br. s., 3H), 3.69 (s, 1H), 3.07-2.85 (m, 3H), 2.71 (br. s., 3H), 2.54 (br. s., 3H), 2.43-2.40 (d, J=12.4 Hz, 1H), 2.01 (br. s., 1H), 1.77-1.59 (m, 3H), 1.54 (br. s., 1H), 1.10-1.07 (d, J=11.91 Hz, 1H).

Isomer B: 1-(1-(2-(4-fluorophenyl)piperidin-4-yl)ethyl)-N-((4-methoxy-6-methyl-2-oxo-1,2-dihydropyridin-3-yl)methyl)-2-methyl-1H-indole-3-carboxamide (7.10 mg, 13.38 umol, 4.24% yield). LCMS m/z 531 [M+H]⁺. ¹H NMR (400 MHz, Methanol-d₄) δ 7.72-7.70 (d, J=7.94 Hz, 2H), 7.42-7.32 (m, 2H), 7.24-7.13 (m, 2H), 7.10-7.06 (t, J=8.49 Hz, 2H), 6.86 (br. s., 1H), 4.54 (br. s., 2H), 4.47-4.38 (m, 1H), 4.08 (s, 3H), 3.63-3.60 (d, J=11.25 Hz, 1H), 2.97-2.88 (m, 2H), 2.82 (s, 1H), 2.67 (s, 3H), 2.51 (s, 3H), 2.38-2.35 (d, J=13.01 Hz, 1H), 1.87-1.80 (m, 1H), 1.77-1.59 (m, 4H), 1.14-1.10 (d, J=13.9 Hz, 1H).

### Example 13: Synthesis of 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide (33)

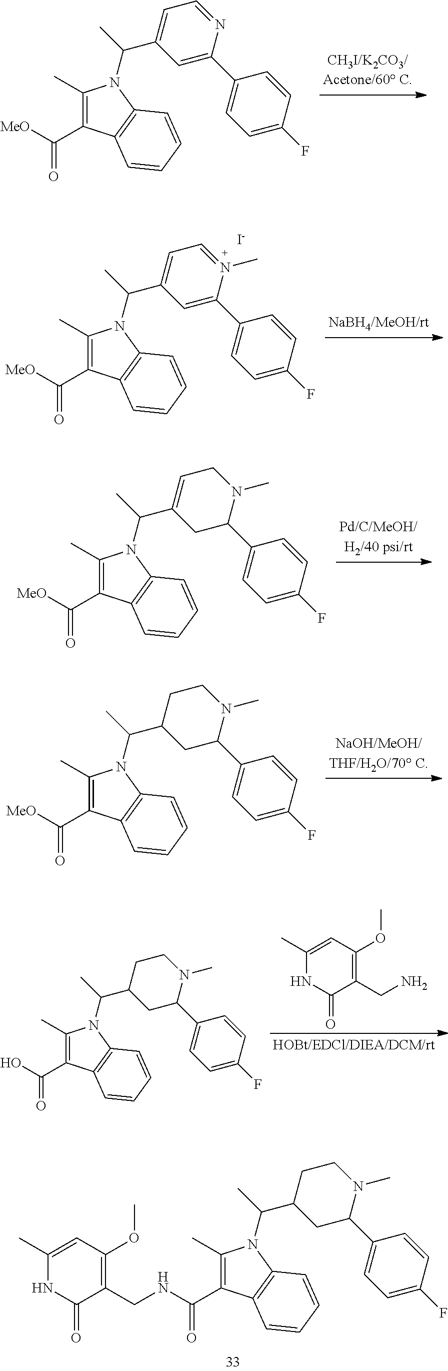

### Step 1: Synthesis of 2-(4-fluorophenyl)-4-(1-(3-(methoxycarbonyl)-2-methyl-1H-indol-1-yl)ethyl)-1-methylpyridin-1-ium iodide

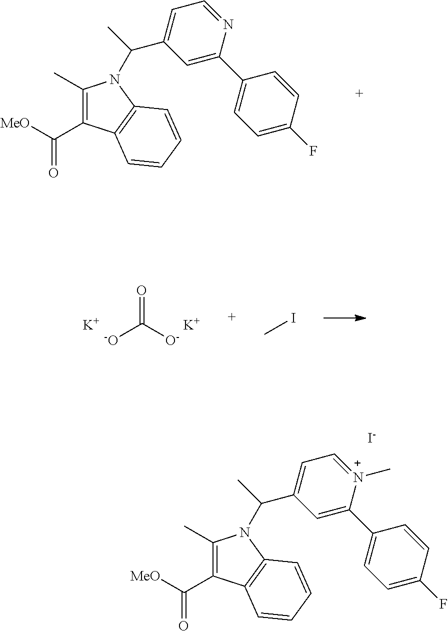

To a solution of methyl 1-(1-(2-(4-fluorophenyl)pyridin-4-yl)ethyl)-2-methyl-1H-indole-3-carboxylate (800.00 mg, 2.06 mmol) in acetone (10 mL) was added potassium carbonate (1.42 g, 10.30 mmol) and iodomethane (2.92 g, 20.60 mmol). The reaction mixture was stirred at 60° C. for 18 hr in a sealed tube. The mixture was diluted with water and extracted with ethyl acetate. The organics layer was concentrated and purified by preparative-TLC (petroleum ether:ethyl acetate=0:1) to afford 2-(4-fluorophenyl)-4-(1-(3-(methoxycarbonyl)-2-methyl-1H-indol-1-yl)ethyl)-1-methylpyridin-1-ium iodide (250.00 mg, 30.08% yield) as a brown solid.

### Step 2: Synthesis of methyl 1-(1-(2-(4-fluorophenyl)-1-methyl-1,2,3,6-tetrahydropyridin-4-yl)ethyl)-2-methyl-1H-indole-3-carboxylate

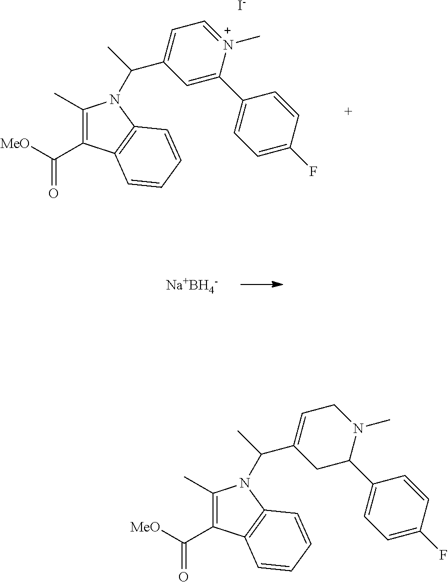 [SMILES: "Na+BH4-" ; [I-] ; COC(=O)c1c(C)n(C(C)c2cc[n+](C)c(-c3ccc(F)cc3)c2)c2ccccc12 ; COC(=O)c1c(C)n(C(C)C2=CCN(C)C(c3ccc(F)cc3)C2)c2ccccc12]

To a solution of 2-(4-fluorophenyl)-4-(1-(3-(methoxycarbonyl)-2-methyl-1H-indol-1-yl)ethyl)-1-methylpyridin-1-ium iodide (230.00 mg, 0.57 mmol) in methanol (5 mL) was added sodium borohydride (172.52 mg, 4.56 mmol). The reaction mixture was stirred at 25° C. for 2 hr. The solvent was removed and the residue was diluted with water and extracted with ethyl acetate. The organics layer was concentrated to afford crude methyl 1-(1-(2-(4-fluorophenyl)-1-methyl-1,2,3,6-tetrahydropyridin-4-yl)ethyl)-2-methyl-1H-indole-3-carboxylate (170.00 mg, 73.36% yield) as a yellow solid.

### Step 3: Synthesis of methyl 1-(1-(2-(4-fluorophenyl)-1-methylpiperidin-4-yl)ethyl)-2-methyl-1H-indole-3-carboxylate

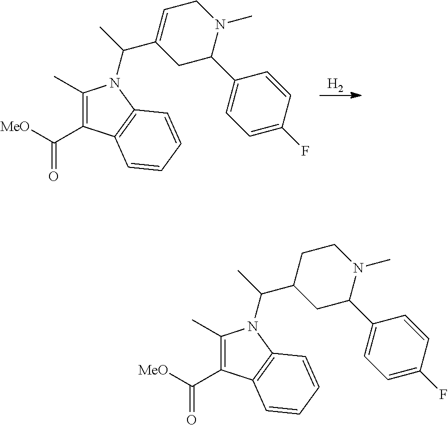

To a solution of methyl 1-(1-(2-(4-fluorophenyl)-1-methyl-1,2,3,6-tetrahydropyridin-4-yl)ethyl)-2-methyl-1H-indole-3-carboxylate (200.00 mg, 0.49 mmol) in methanol (4 mL) was added Pd/C (200 mg). The reaction mixture was stirred at room temperature under an atmosphere of H₂ (40 psi) for 24 hr. The mixture was filtered and the filtrate was concentrated. The crude residue was purified by preparative-TLC (petroleum ether:ethyl acetate=2:1) to afford methyl 1-(1-(2-(4-fluorophenyl)-1-methylpiperidin-4-yl)ethyl)-2-methyl-1H-indole-3-carboxylate (30.00 mg, 14.93% yield) as a yellow solid.

### Step 4: Synthesis of 1-(1-(2-(4-fluorophenyl)-1-methylpiperidin-4-yl)ethyl)-2-methyl-1H-indole-3-carboxylic acid

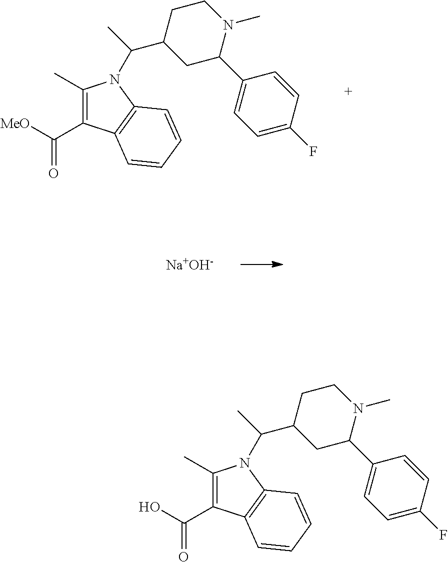 [SMILES: "Na+OH-" ; COC(=O)c1c(C)n(C(C)C2CCN(C)C(c3ccc(F)cc3)C2)c2ccccc12 ; Cc1c(C(=O)O)c2ccccc2n1C(C)C1CCN(C)C(c2ccc(F)cc2)C1]

To a solution of methyl 1-(1-(2-(4-fluorophenyl)-1-methylpiperidin-4-yl)ethyl)-2-methyl-1H-indole-3-carboxylate (30.00 mg, 0.07 mmol) in methanol (1 mL), water (1 mL) and tetrahydrofuran (1 mL) was added sodium hydroxide (29.38 mg, 0.73 mmol) and the solution was stirred at 70° C. for 12 hr. The solution was cooled to room temperature and the volatiles were removed. The resulting residue was dissolved in water and the resulting solution was acidified by addition of hydrogen chloride (2M) to pH=6. The aqueous solution was lyophilized to afford 1-(1-(2-(4-fluorophenyl)-1-methylpiperidin-4-yl)ethyl)-2-methyl-1H-indole-3-carboxylic acid (30 mg, crude) as a white solid.

### Step 5: Synthesis of 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide

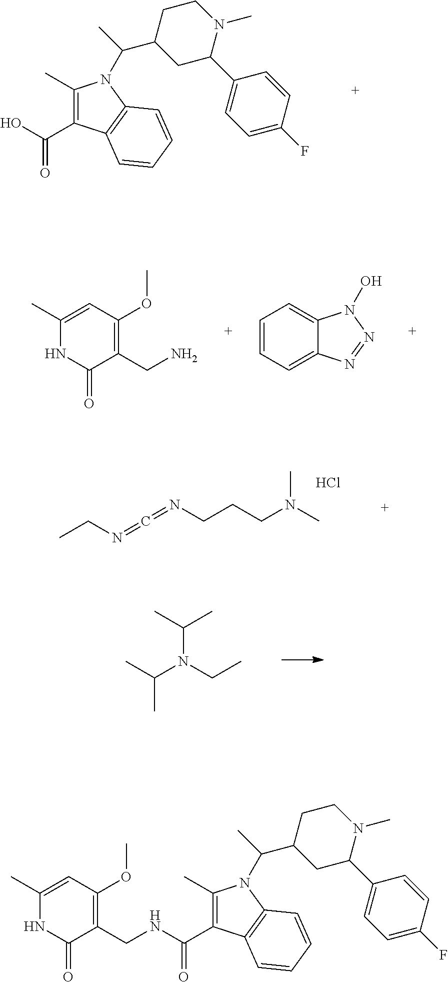

To a solution of 1-(1-(2-(4-fluorophenyl)-1-methylpiperidin-4-yl)ethyl)-2-methyl-1H-indole-3-carboxylic acid (30.00 mg, 76.05 umol) in dichloromethane (2 mL) was added 1H-benzo[d][1,2,3]triazol-1-ol (20.55 mg, 152.10 umol), N1-((ethylimino)methylene)-N3,N3-dimethylpropane-1,3-diamine hydrochloride (29.16 mg, 152.10 umol), N-ethyl-N-isopropylpropan-2-amine (29.49 mg, 228.15 umol) and 3-(aminomethyl)-4-methoxy-6-methylpyridin-2(1H)-one (25.58 mg, 152.10 umol). The reaction mixture was stirred at room temperature for 18 hr. The mixture was diluted with water and extracted with dichloromethane. The organic layer was concentrated and the crude product was purified by preparative-HPLC (Instrument: gilson GX281 Column: Grace C18 150*25 mm*5 um Mobile phase A: water with 0.1% hydrogen chloride solution Mobile phase B: acetonitrile column temperature: 40° C. Gradient 11-41% B22 min) to afford 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide (3.20 mg, 7.73% yield) as a brown solid. LCMS m/z 545 [M+H]⁺.

¹H NMR (400 MHz, Methanol-d₄) δ 7.78-7.76 (d, J=7.34 Hz, 1H), 7.71-7.69 (d, J=7.58 Hz, 1H), 7.65 (br. s., 2H), 7.31-7.27 (t, J=8.44 Hz, 2H), 7.23-7.15 (m, 2H), 6.83 (br. s., 1H), 4.45-4.37 (m, 1H), 4.32-4.29 (d, J=11.74 Hz, 1H), 4.10 (s, 3H), 3.49-3.46 (d, J=11.98 Hz, 1H), 3.07-2.98 (m, 1H), 2.90 (s, 3H), 2.70 (s, 3H), 2.52-2.51 (d, J=3.91 Hz, 5H), 2.44-2.41 (d, J=12.96 Hz, 1H), 2.15-2.03 (m, 2H), 1.66-1.64 (d, J=6.60 Hz, 3H), 1.30 (br. s., 1H), 1.16-1.13 (d, J=14.67 Hz, 1H).

### Example 14: Synthesis of 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide Isomer A (34) and Isomer B (35)

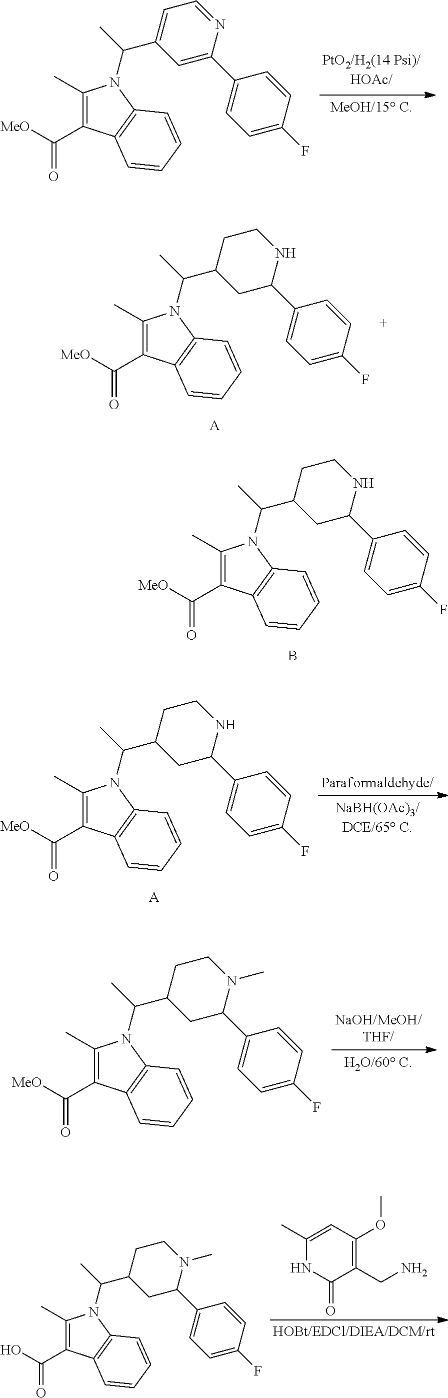

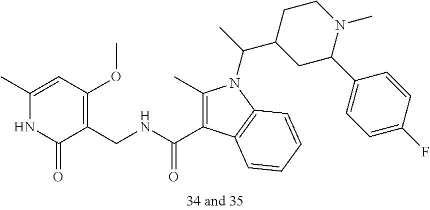 [SMILES: COc1cc(C)[nH]c(=O)c1CNC(=O)c1c(C)n(C(C)C2CCN(C)C(c3ccc(F)cc3)C2)c2ccccc12]

### Step 1: Synthesis of methyl 1-(1-(2-(4-fluorophenyl)piperidin-4-yl)ethyl)-2-methyl-1H-indole-3-carboxylate

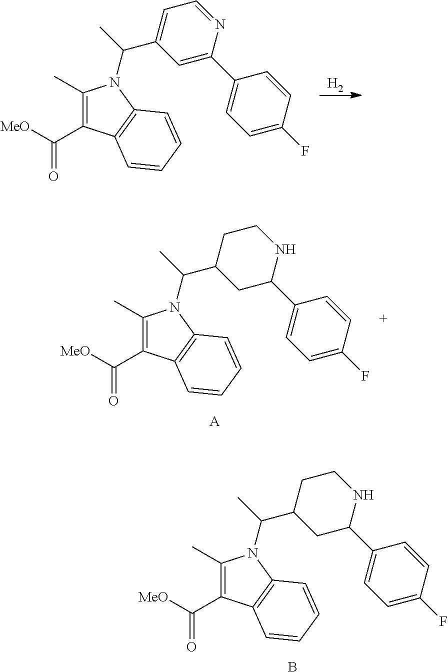

To a solution of methyl 1-(1-(2-(4-fluorophenyl)pyridin-4-yl)ethyl)-2-methyl-1H-indole-3-carboxylate (1.5 g, 3.87 mmol) in methanol (24 mL) and acetic acid (3 mL) was added platinum(IV) oxide (1.35 g, 6 mmol) and the reaction was stirred under an atmosphere of H₂ (1 atm) at room temperature for 22 hr. The solution was filtered and the filtrate was concentrated. The crude product was purified by preparative-HPLC (Instrument: gilson GX281 Column: Grace C18 150*25 mm*5 um Mobile phase A: water with 0.1% hydrogen chloride solution Mobile phase B: acetonitrile column temperature: 40° C. Gradient 30-60% B 22 min) to afford two isomers. The two isomers were processed through the subsequent steps separately.

Isomer A: methyl 1-(1-(2-(4-fluorophenyl)piperidin-4-yl)ethyl)-2-methyl-1H-indole-3-carboxylate (150 mg, 10% yield)

Isomer B: methyl 1-(1-(2-(4-fluorophenyl)piperidin-4-yl)ethyl)-2-methyl-1H-indole-3-carboxylate (450 mg, 29.5% yield) as yellow oil.

### Step 2: Synthesis of methyl 1-(1-(2-(4-fluorophenyl)-1-methylpiperidin-4-yl)ethyl)-2-methyl-1H-indole-3-carboxylate

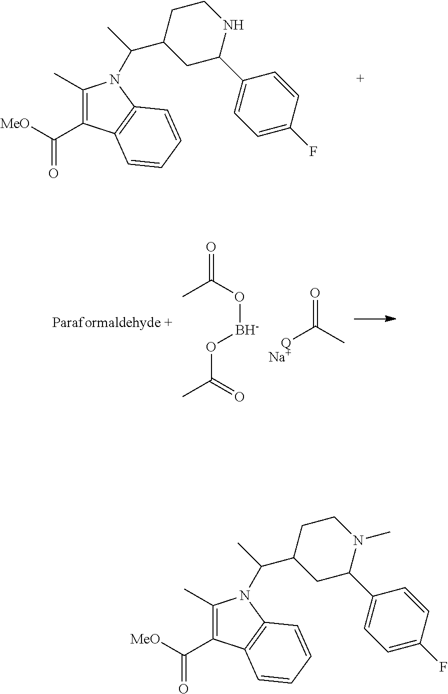

To a solution of methyl 1-(1-(2-(4-fluorophenyl)piperidin-4-yl)ethyl)-2-methyl-1H-indole-3-carboxylate (Isomer A: 150 mg, 380.25 umol) in 1,2-dichloroethane (3 mL) was added paraformaldehyde (68.51 mg, 760.50 umol). The reaction mixture was stirred at 70° C. for 30 min before cooling the reaction to room temperature and addition of sodium triacetoxyborohydride (241.77 mg, 1.14 mmol). The reaction was then heated to 70° C. and stirred for 4 hours. The mixture was cooled to room temperature, filtered, and the filtrate was diluted with water and extracted with ethyl acetate. The organic layer was concentrated to afford crude product methyl 1-(1-(2-(4-fluorophenyl)-1-methylpiperidin-4-yl)ethyl)-2-methyl-1H-indole-3-carboxylate (150 mg, 96.57% yield) as a white solid.

### Step 3: Synthesis of 1-(1-(2-(4-fluorophenyl)-1-methylpiperidin-4-yl)ethyl)-2-methyl-1H-indole-3-carboxylic Acid

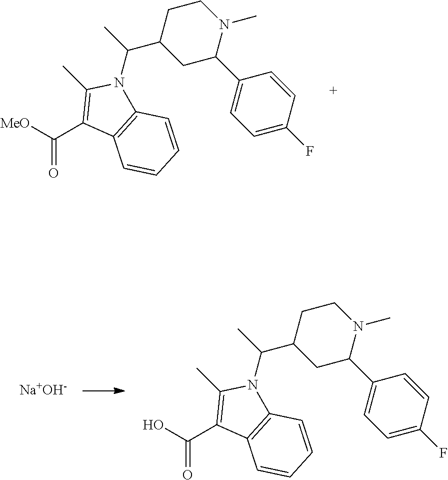 [SMILES: "Na+OH-" ; COC(=O)c1c(C)n(C(C)C2CCN(C)C(c3ccc(F)cc3)C2)c2ccccc12 ; Cc1c(C(=O)O)c2ccccc2n1C(C)C1CCN(C)C(c2ccc(F)cc2)C1]

To a solution of methyl 1-(1-(2-(4-fluorophenyl)-1-methylpiperidin-4-yl)ethyl)-2-methyl-1H-indole-3-carboxylate (150 mg, 367.19 umol) in methanol (1 mL), water (1 mL), and tetrahydrofuran (1 mL) was added sodium hydroxide (146 mg, 3.67 mmol) at 70° C. The solution was stirred for 28 hours. The solution was cooled to room temperature and concentrated. Water was added to the residue and the resulting solution was acidified by addition of hydrogen chloride (2M) to pH=6. The aqueous solution was extracted with ethyl acetate and the organic layer was concentrated to afford crude 1-(1-(2-(4-fluorophenyl)-1-methylpiperidin-4-yl)ethyl)-2-methyl-1H-indole-3-carboxylic acid (150.00 mg, 99% yield) as a white solid.

### Step 4: Synthesis of 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide

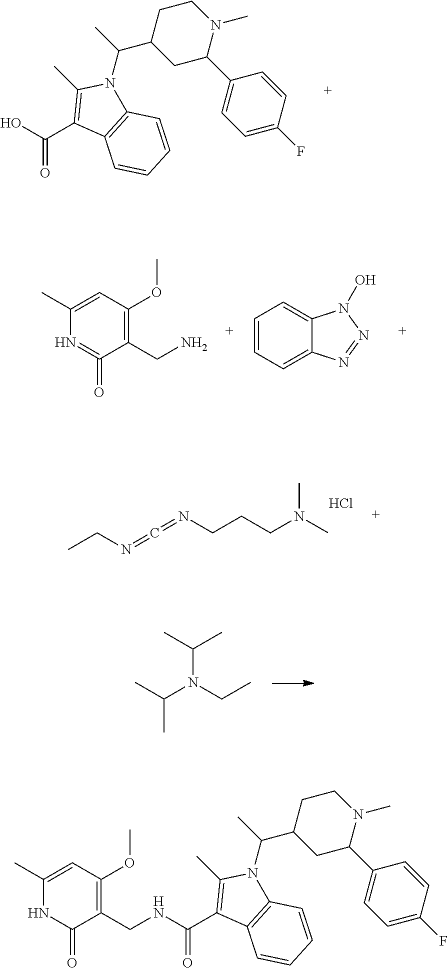

To a solution of 1-(1-(2-(4-fluorophenyl)-1-methylpiperidin-4-yl)ethyl)-2-methyl-1H-indole-3-carboxylic acid (150 mg, 380.25 umol) in dichloromethane (3 mL) were added 1H-benzo[d][1,2,3]triazol-1-ol (102.76 mg, 760.49 umol), N1-((ethylimino)methylene)-N3,N3-dimethylpropane-1,3-diamine hydrochloride (145.79 mg, 760.49 umol), N-ethyl-N-isopropylpropan-2-amine (147.43 mg, 1.14 mmol) and 3-(aminomethyl)-4-methoxy-6-methyl-1H-pyridin-2-one (127.91 mg, 625.01 umol). The reaction mixture was stirred at room temperature for 18 h. The mixture was diluted with water and extracted with dichloromethane. The organic layer was purified by preparative-HPLC (Instrument: gilson GX281 Column: Grace C18 150*25 mm*5 um Mobile phase A: water with 0.1% hydrogen chloride solution Mobile phase B: acetonitrile column temperature: 40 C Gradient 12-42% B 22 min) to afford 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide Isomer A (5.00 mg, 2.41% yield) as a green solid. LCMS m/z 545 [M+H]⁺. ¹H NMR (400 MHz, Methanol-d₄) δ 7.74-7.65 (m, 2H), 7.41 (br. s., 2H), 7.24-7.14 (m, 2H), 7.14-7.07 (m, 2H), 6.89 (s, 1H), 4.46-4.38 (m, 1H), 4.14-4.00 (m, 4H), 3.79-3.76 (d, J=11.91 Hz, 1H), 3.39-3.31 (m, 2H), 2.95-2.93 (d, J=10.8 Hz, 1H), 2.65 (s, 3H), 2.52 (s, 6H), 2.41-2.38 (d, J=13.7 Hz, 1H), 2.08-1.97 (d, J=13.0 Hz, 2H), 1.87-1.77 (m, 1H), 1.71-1.70 (d, J=6.8 Hz, 3H), 1.14-1.10 (d, J=13.5 Hz, 1H).

Isomer B of 1-(1-(2-(4-fluorophenyl)-1-methylpiperidin-4-yl)ethyl)-N-((4-methoxy-6-methyl-2-oxo-1,2-dihydropyridin-3-yl)methyl)-2-methyl-1H-indole-3-carboxamide was synthesized using isomer B in Step 1. LCMS m/z 545 [M+H]⁺; ¹H NMR (400 MHz, Methanol-d₄): δ7.77-7.65 (m, 2H), 7.62-7.60 (d, J=7.94 Hz, 1H), 7.45-7.37 (m, 2H), 7.15-7.08 (m, 3H), 6.29 (s, 1H), 4.54 (s, 2H), 4.28-4.19 (m, 1H), 3.95 (s, 3H), 3.12 (br. s., 1H), 2.92-2.89 (d, J=11.91 Hz, 1H), 2.63 (s, 3H), 2.56-2.53 (d, J=11.3 Hz, 1H), 2.33 (s, 3H), 2.10-2.07 (d, J=11.47 Hz, 2H), 2.00 (s, 3H), 1.65-1.48 (m, 4H), 1.31-1.29 (d, J=11.9 Hz, 1H), 0.97-0.94 (d, J=12.1 Hz, 1H).

### Example 15: Synthesis of N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer A (12) and Isomer B (14)

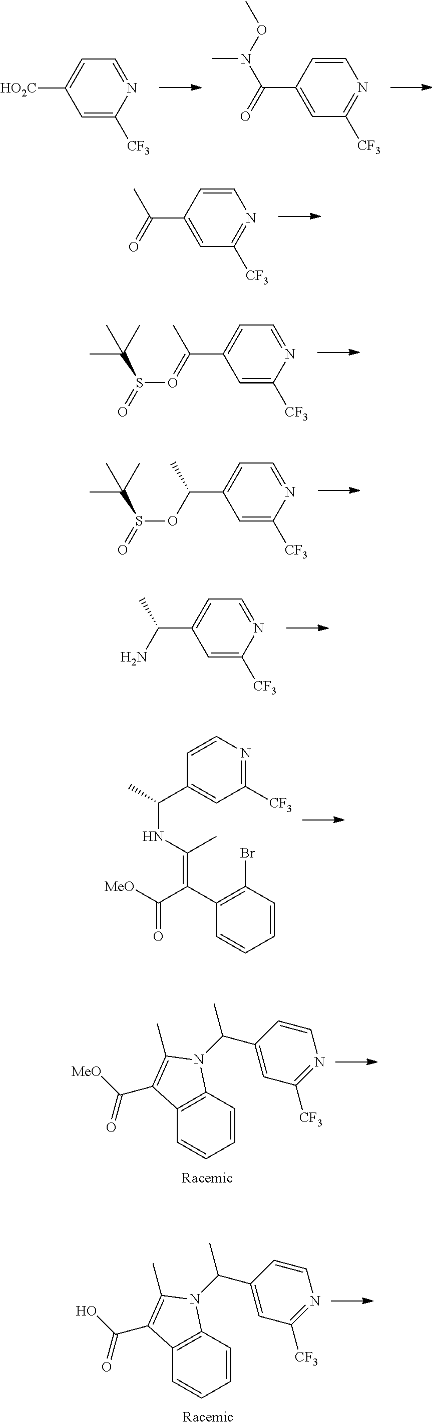

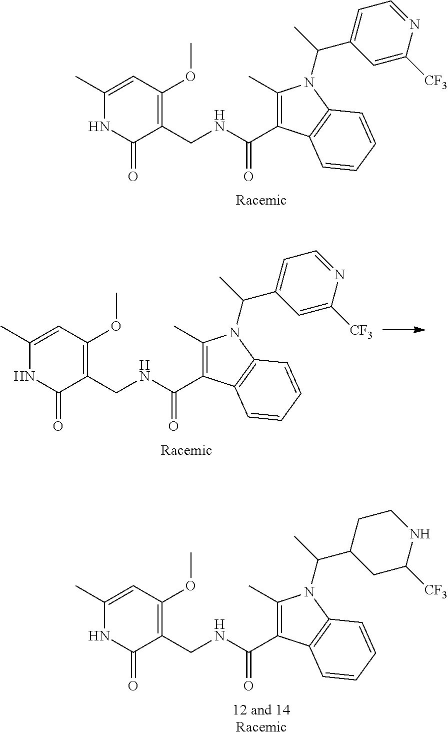 [SMILES: COc1cc(C)[nH]c(=O)c1CNC(=O)c1c(C)n(C(C)c2ccnc(C(F)(F)F)c2)c2ccccc12 ; COc1cc(C)[nH]c(=O)c1CNC(=O)c1c(C)n(C(C)c2ccnc(C(F)(F)F)c2)c2ccccc12 ; COc1cc(C)[nH]c(=O)c1CNC(=O)c1c(C)n(C(C)C2CCNC(C(F)(F)F)C2)c2ccccc12]

### Synthesis of N-methoxy-N-methyl-2-(trifluoromethyl)isonicotinamide

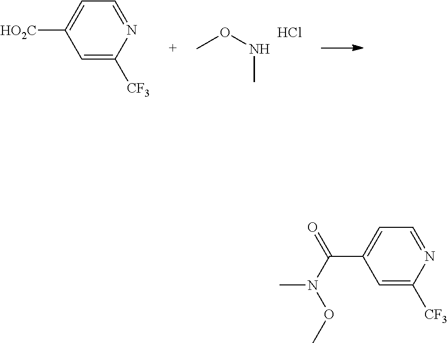

To a mixture of 2-(trifluoromethyl)isonicotinic acid (50.0 g, 261.6 mmol) in CH₂Cl₂ (500 mL) were added DIEA (118.4 g, 915.7 mmol), HATU (149.2 g, 392.4 mmol) and N,O-dimethylhydroxylamine hydrochloride (51.0 g, 523.3 mmol) at 0° C. The mixture was stirred at 25° C. for 15 h. The reaction mixture was cooled to 0° C. was quenched with water (200 mL) and resultant bi-phasic mixture was extracted with EtOAc (3×300 mL). The combined organic layers were dried over anhydrous Na₂SO₄ and concentrated in vacuo to give crude product. The crude product was purified by silica gel chromatography (petroleum ether/EtOAc=15:1 to 3:1) to give N-methoxy-N-methyl-2-(trifluoromethyl)isonicotinamide (60.0 g, 255.0 mmol, 98% yield) as a yellow oil. Four additional batches of N-methoxy-N-methyl-2-(trifluoromethyl)isonicotinamide were synthesized in parallel on similar scale using the procedure outlined above.

### Synthesis of 1-(2-(trifluoromethyl)pyridin-4-yl)ethanone

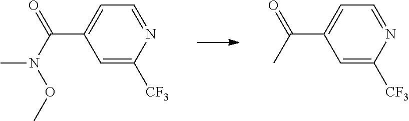 [SMILES: CON(C)C(=O)c1ccnc(C(F)(F)F)c1 ; CC(=O)c1ccnc(C(F)(F)F)c1]

To a mixture of N-methoxy-N-methyl-2-(trifluoromethyl)isonicotinamide (60.0 g, 256.2 mmol) in THF (500 mL) was added a solution of MeMgBr (256 mL, 3 M in diethyl ether) dropwise at −70° C. under N₂. The mixture was stirred at 0° C. for 2 h. The mixture was quenched with saturated aqueous NH₄Cl (200 mL) at 0° C. and extracted with EtOAc (3×200 mL). The combined organic layers were dried over anhydrous Na₂SO₄ and concentrated in vacuo to give 1-(2-(trifluoromethyl)pyridin-4-yl)ethanone (37.5 g), was directly used in the subsequent reaction without further purification. Four additional batches of 1-(2-(trifluoromethyl)pyridin-4-yl)ethanone were synthesized in parallel on similar scale using the procedure outlined above.

### Synthesis of (R)-2-methyl-N-(1-(2-(trifluoromethyl)pyridin-4-yl)ethylidene)propane-2-sulfinamide

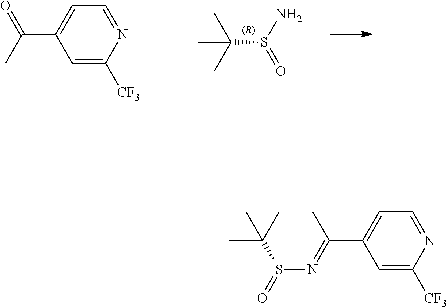

To a mixture of 1-(2-(trifluoromethyl)pyridin-4-yl)ethanone (50.0 g, 264.4 mmol) and (R)-2-methylpropane-2-sulfinamide (48.1 g, 396.6 mmol) in THF (250 mL) was added Ti(OEt)₄ (90.5 g, 396.6 mmol) in one portion. The mixture was stirred at 80° C. for 15 h. The mixture was quenched with water (200 mL) at 25° C. and extracted with EtOAc (3×200 mL). The combined organic layers were dried over anhydrous Na₂SO₄ and concentrated in vacuo. The crude product was purified by silica gel chromatography (petroleum ether/EtOAc=15:1 to 5:1) to give (R)-2-methyl-N-(1-(2-(trifluoromethyl)pyridin-4-yl)ethylidene)propane-2-sulfinamide (52.3 g, 179.0 mmol, 67.7% yield) as a yellow oil. Three additional batches of (R)-2-methyl-N-(1-(2-(trifluoromethyl)pyridin-4-yl)ethylidene)propane-2-sulfinamide were synthesized in parallel on similar scale using the procedure outlined above.

### Synthesis of (R)-2-methyl-N-(1-(2-(trifluoromethyl)pyridin-4-yl)ethyl)propane-2-sulfinamide

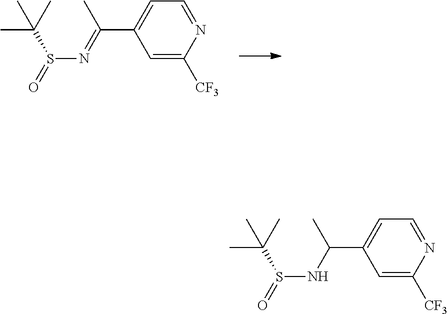 [SMILES: C/C(=N\[S@](=O)C(C)(C)C)c1ccnc(C(F)(F)F)c1 ; CC(N[S@](=O)C(C)(C)C)c1ccnc(C(F)(F)F)c1]

To a mixture of (R)-2-methyl-N-(1-(2-(trifluoromethyl)pyridin-4-yl)ethylidene)propane-2-sulfinamide (300 mg, 1.03 mmol) in THF (5 mL) and water (0.1 mL) was added NaBH₄ (117 mg, 3.09 mmol) at −78° C. The reaction mixture was stirred at room temperature for 3 h. To the reaction mixture was added water and the aqueous layer was extracted with EtOAc (3×10 mL). The combined organic layers were dried over Na₂SO₄ and concentrated to give the crude product (R)-2-methyl-N-(1-(2-(trifluoromethyl)pyridin-4-yl)ethyl)propane-2-sulfinamide (300 mg, dr ˜5:1) as a yellow oil. LCMS (M+H)⁺ m/z: calcd. 295.1, found 295.1.

### Synthesis of 1-(2-(trifluoromethyl)pyridin-4-yl)ethan-1-amine

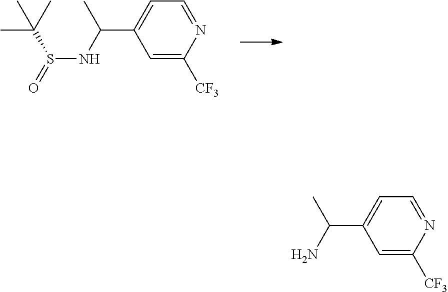 [SMILES: CC(N[S@](=O)C(C)(C)C)c1ccnc(C(F)(F)F)c1 ; CC(N)c1ccnc(C(F)(F)F)c1]

To a solution of (R)-2-methyl-N-(1-(2-(trifluoromethyl)pyridin-4-yl)ethyl)propane-2-sulfinamide (300 mg, 1.02 mmol) in MeOH (3 mL) was added a solution of HCl (1 mL, 4N in MeOH). The reaction mixture was stirred at room temperature for 2 h. The resulting reaction mixture was adjusted to pH-9 with Et₃N and concentrated to give crude product 1-(2-(trifluoromethyl)pyridin-4-yl)ethan-1-amine (180 mg, 93.2% yield) as a yellow solid.

### Synthesis of methyl 2-(2-bromophenyl)-3-((1-(2-(trifluoromethyl)pyridin-4-yl)ethyl)amino)but-2-enoate

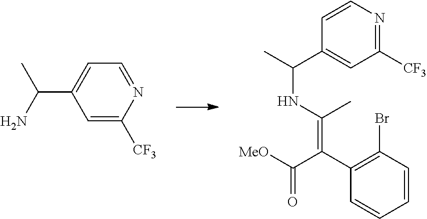 [SMILES: CC(N)c1ccnc(C(F)(F)F)c1 ; COC(=O)/C(=C(/C)NC(C)c1ccnc(C(F)(F)F)c1)c1ccccc1Br]

To a solution of 1-(2-(trifluoromethyl)pyridin-4-yl)ethan-1-amine (180 mg, 0.95 mmol) and methyl 2-(2-bromophenyl)-3-oxobutanoate (256 mg, 0.95 mmol) in EtOH (5 mL) was added AcOH (170 mg, 2.84 mmol). The reaction mixture was stirred overnight at 120° C. The reaction was completed and concentrated. The resulting oil was extracted with EtOAc (3×10 mL), and the crude product was purified by TLC (petroleum ether/EtOAc=5:1) to afford methyl 2-(2-bromophenyl)-3-((1-(2-(trifluoromethyl)pyridin-4-yl)ethyl)amino)but-2-enoate (300 mg, 71.5% yield) as a colorless oil.

### Synthesis of Methyl 2-methyl-1-(1-(2-(trifluoromethyl)pyridin-4-yl)ethyl)-1H-indole-3-carboxylate

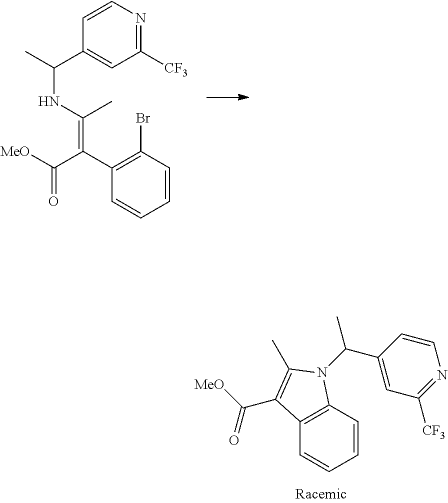 [SMILES: COC(=O)/C(=C(/C)NC(C)c1ccnc(C(F)(F)F)c1)c1ccccc1Br ; COC(=O)c1c(C)n(C(C)c2ccnc(C(F)(F)F)c2)c2ccccc12]

To a mixture methyl 2-(2-bromophenyl)-3-((1-(2-(trifluoromethyl)pyridin-4-yl)ethyl)amino)but-2-enoate (120 mg, 0.27 mmol) in 1,4dioxane (3 mL) were added NaOMe (44 mg, 0.81 mmol), dicyclohexyl(2′,6′-diisopropoxy-[1,1′-biphenyl]-2-yl)phosphine (6.3 mg, 0.014 mmol), and methanesulfonato(2-dicyclohexylphosphino-2′,6′-di-i-propoxy-1,1′-biphenyl)(2-amino-1-biphenyl-2-1)palladium (11.3 mg, 0.014 mmol). The reaction mixture was stirred at 120° C. for 2 h. The reaction was completed and the mixture was purified by TLC (petroleum ether/EtOAc=2:1) to give methyl 2-methyl-1-(1-(2-(trifluoromethyl)pyridin-4-yl)ethyl)-1H-indole-3-carboxylate (50 mg, 50.9% yield) as a white solid.

### Synthesis of 2-methyl-1-(1-(2-(trifluoromethyl)pyridin-4-yl)ethyl)-1H-indole-3-carboxylic Acid

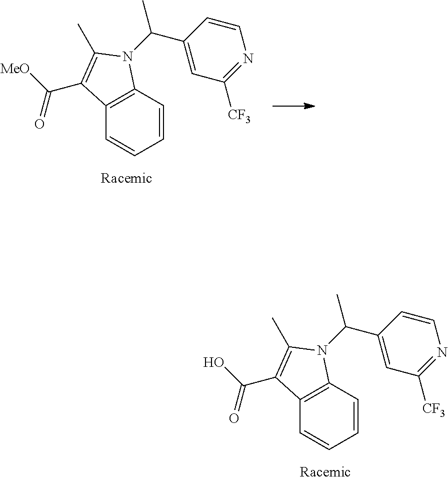 [SMILES: COC(=O)c1c(C)n(C(C)c2ccnc(C(F)(F)F)c2)c2ccccc12 ; Cc1c(C(=O)O)c2ccccc2n1C(C)c1ccnc(C(F)(F)F)c1]

To a mixture of methyl 2-methyl-1-(1-(2-(trifluoromethyl)pyridin-4-yl)ethyl)-1H-indole-3-carboxylate (50 mg, 0.14 mmol) in MeOH (3 mL) and water (1 mL) was added NaOH (16.5 mg, 0.42 mmol). The reaction mixture was stirred overnight at reflux. The reaction mixture was concentrated, and the mixture was acidified to pH-6 with aqueous 2N HCl. The aqueous layer was extracted with EtOAc (3×10 mL). The organic layer was dried over Na₂SO₄ and concentrated to give the crude product 2-methyl-1-(1-(2-(trifluoromethyl)pyridin-4-yl)ethyl)-1H-indole-3-carboxylic acid (30 mg, 62.5% yield) as a yellow solid.

### Synthesis of N-((4-methoxy-6-methyl-2-oxo-1,2-dihydropyridin-3-yl)methyl)-2-methyl-1-(1-(2-(trifluoromethyl)pyridin-4-yl)ethyl)-1H-indole-3-carboxamide

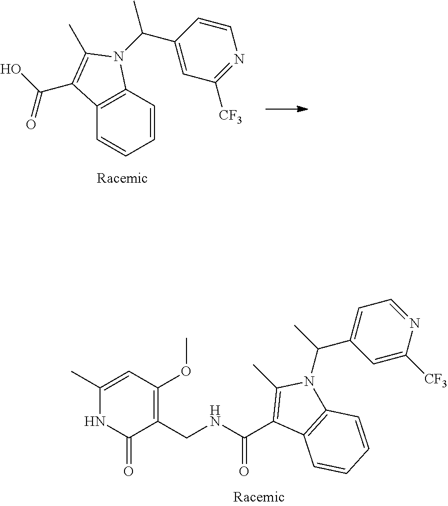 [SMILES: Cc1c(C(=O)O)c2ccccc2n1C(C)c1ccnc(C(F)(F)F)c1 ; COc1cc(C)[nH]c(=O)c1CNC(=O)c1c(C)n(C(C)c2ccnc(C(F)(F)F)c2)c2ccccc12]

To a solution of 2-methyl-1-(1-(2-(trifluoromethyl)pyridin-4-yl)ethyl)-1H-indole-3-carboxylic acid (30 mg, 0.08 mmol) and 3-(aminomethyl)-4-methoxy-6-methylpyridin-2(1H)-one hydrochloride (21 mg, 0.1 mmol) in CH₂Cl₂ (3 mL) were added 2-(3H-[1,2,3]triazolo[4,5-b]pyridin-3-yl)-1,1,3,3-tetramethylisouronium hexafluorophosphate(V) (49 mg, 0.12 mmol) and DIEA (33.3 mg, 0.25 mmol). The mixture was stirred overnight at room temperature. The reaction mixture was concentrated and purified by preparative HPLC (Mobile phase A: water with 0.05% ammonia solution; Mobile phase B: CH₃CN; column temperature: 28° C., Gradient: 35-65% B over 10 min) to give product N-((4-methoxy-6-methyl-2-oxo-1,2-dihydropyridin-3-yl)methyl)-2-methyl-1-(1-(2-(trifluoromethyl)pyridin-4-yl)ethyl)-1H-indole-3-carboxamide (10 mg, 23.3% yield) as a white solid. ¹H NMR (400 MHz, CDCl₃) δ 12.72 (s, 1H), 8.63-8.62 (m, 1H), 7.90-7.88 (m, 1H), 7.73-7.72 (m, 1H), 7.50 (s, 1H), 7.13-7.12 (d, J=4 Hz, 1H), 7.03 (m, 2H), 6.81-9.79 (m, 1H), 5.93 (s, 1H), 5.81 (m, 1H), 4.71 (m, 2H), 3.91 (s, 3H), 2.74 (s, 3H), 2.16 (s, 3H), 2.01-1.99 (d, J=8 Hz, 3H); LCMS (M+H)⁺ m/z: calcd. 499.5, found 499.2.

### Synthesis of N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide

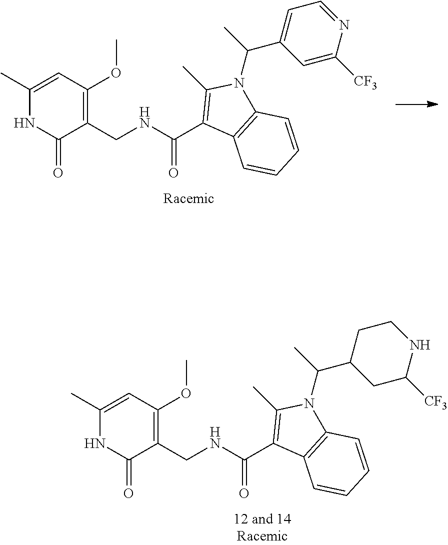 [SMILES: COc1cc(C)[nH]c(=O)c1CNC(=O)c1c(C)n(C(C)c2ccnc(C(F)(F)F)c2)c2ccccc12 ; COc1cc(C)[nH]c(=O)c1CNC(=O)c1c(C)n(C(C)C2CCNC(C(F)(F)F)C2)c2ccccc12]

To a solution of N-((4-methoxy-6-methyl-2-oxo-1,2-dihydropyridin-3-yl)methyl)-2-methyl-1-(1-(2-(trifluoromethyl)pyridin-4-yl)ethyl)-1H-indole-3-carboxamide (180 mg, 0.36 mmol) in MeOH (1 mL) was added platinum(IV) oxide (100 mg) and AcOH (3 mL). The reaction mixture was stirred under 50 psi of H₂ (g) at 30° C. for 12 h. The reaction mixture was filtered over a pad of Celite and the filtrate was concentrated. The crude mixture was purified by HPLC (Mobile phase A: water with 0.05% ammonia solution; Mobile phase B: acetonitrile; column temperature: 40° C., Gradient: 46-76% B 10 min) to afford two separated diastereomers of N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide each as a white solids.

Isomer A (12): (30 mg, 16.4% yield); ¹H NMR (400 MHz, CDCl₃) δ 12.55 (s, 1H), 7.88-7.86 (m, 1H), 7.55 (s, 1H), 7.40-7.38 (m, 1H), 7.10-6.99 (m, 2H), 5.90 (s, 1H), 4.72-4.63 (m, 2H), 4.15-4.11 (m, 1H), 3.89 (s, 3H), 3.32-3.29 (d, J=12 Hz, 1H), 2.81 (m, 5H), 2.39-2.36 (m, 1H), 2.14 (s, 3H), 2.01-1.98 (d, J=12 Hz, 1H), 1.57 (s, 3H), 1.27 (m, 2H), 0.97 (m, 2H); LCMS (M+H)⁺ calcd. 505.6; found 505.2.

Isomer B (14): (15 mg, 8.2% yield); ¹H NMR (400 MHz, Methanol-d₄) δ 7.62-7.60 (m, 1H), 7.52 (m, 1H), 7.04-6.97 (m, 2H), 6.18 (s, 1H), 4.43 (s, 2H), 4.19-4.11 (m, 1H), 3.85 (s, 3H), 2.84-2.81 (m, 1H), 2.51 (m, 1H and s, 3H), 2.41 (m, 1H), 2.38 (m, 1H), 2.22 (s, 3H), 1.54 (d, J=8 Hz, 1H), 1.25-1.22 (m, 3H), 0.90-0.76 (m, 3H); LCMS (M+H)⁺ calcd. 505.6; found 505.2.

### Example 16: Synthesis of N-((4,6-dimethyl-2-oxo-1,2-dihydropyridin-3-yl)methyl)-2-methyl-1-(1-(2-(trifluoromethyl)piperidin-4-yl)ethyl)-1H-indole-3-carboxamide Isomer A (16) and Isomer B (17)

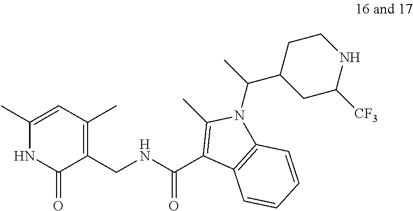 [SMILES: Cc1cc(C)c(CNC(=O)c2c(C)n(C(C)C3CCNC(C(F)(F)F)C3)c3ccccc23)c(=O)[nH]1]

The title compounds were synthesized using similar protocol described in Example 15 above starting from common the intermediate 2-methyl-1-(1-(2-(trifluoromethyl)pyridin-4-yl)ethyl)-1H-indole-3-carboxylic acid.

Isomer A: ¹H NMR (400 MHz, Methanol-d₄) δ 7.73-7.71 (m, 1H), 7.58-7.56 (m, 1H), 7.14-7.07 (m, 2H), 6.10 (s, 1H), 4.52 (s, 2H), 4.24-4.18 (m, 1H), 3.21-3.17 (m, 1H), 2.97 (br. s, 1H), 2.73-2.67 (m, 1H and s, 3H), 2.49-2.46 (m, 1H), 2.40 (s, 3H), 2.23 (s, 3H), 2.03-2.00 (m, 1H), 1.60 (d, J=6.8 Hz, 3H), 1.31-1.26 (m, 1H), 1.07-0.93 (m, 2H); LCMS (M+H)⁺ calcd. 489.6; found 489.1.

Isomer B: ¹H NMR (400 MHz, Methanol-d₄) δ 7.72-7.70 (m, 1H), 7.59-7.57 (m, 1H), 7.12-7.06 (m, 2H), 6.10 (s, 1H), 4.51 (s, 2H), 4.25-4.21 (m, 1H), 2.90-2.87 (m 1H), 2.71-2.61 (m, 1H and s, 3H), 2.59-2.48 (m, 1H), 2.40-2.34 (s, 3H and m, 1H), 2.23-2.17 (s, 3H and m, 1H), 1.62 (d, J=8 Hz, 3H), 1.29-1.23 (m, 1H), 0.99-0.95 (m, 1H), 0.93-0.79 (m, 1H); LCMS (M+H)⁺ calcd. 489.6; found 489.

### Example 17: Synthesis of N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide Isomer A (13)

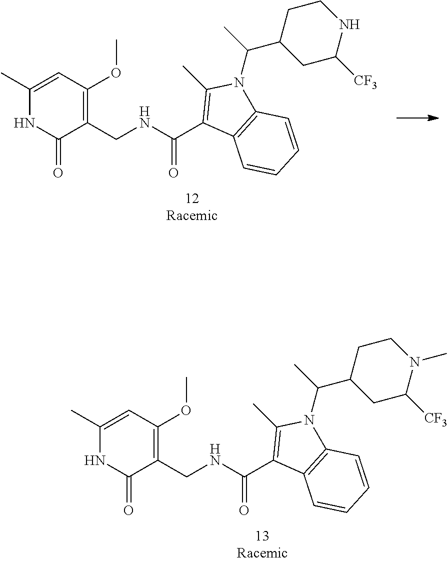

To a mixture N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer A, compound 12 (27 mg, 0.05 mmol) in THF (3 mL) were added a solution of 37% formaldehyde (13 mg, 0.4 mmol) and sodium triacetoxyhydroborate (113 mg, 0.500 mmol). The reaction mixture was stirred at 50° C. overnight. The reaction mixture was quenched by the addition of saturated aqueous K₂CO₃. The aqueous layer was extracted with EtOAc (3×30 mL). The organic layer was dried over Na₂SO₄ and concentrated. The crude product was purified by HPLC (Mobile phase A: water with 0.05% ammonia solution; Mobile phase B: acetonitrile; column temperature: 40° C., Gradient: 40-60% B over 10 min) to give N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide Isomer A (2.3 mg, 8.3% yield) as a white solid. ¹H NMR (400 MHz, Methanol-d₄) δ 7.72-7.70 (m, 1H), 7.57-7.55 (m, 1H), 7.14-7.07 (m, 2H), 6.26 (s, 1H), 4.52 (s, 2H), 4.25-4.20 (m, 1H), 3.93 (s, 3H), 3.03-3.00 (m, 1H), 2.59 (s, 3H), 2.48-2.41 (m, 3H), 2.35 (s, 3H), 2.31 (s, 3H), 2.02-1.98 (m, 1H), 1.60 (d, J=6.8 Hz, 3H), 1.49-1.43 (m, 1H), 1.13-1.03 (m, 2H); LCMS (M+H)⁺ calcd. 519.6; found 519.2.

### Example 18: Synthesis of N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide Isomer B (15)

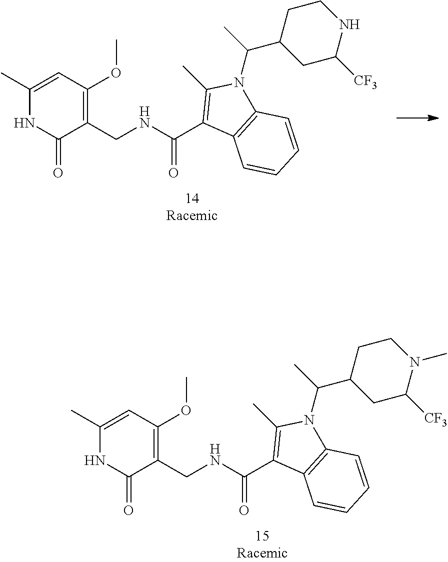

To a mixture N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer B, compound 14 (10 mg, 0.02 mmol) in THF (3 mL) were added a solution of 37% formaldehyde (4.8 mg, 0.06 mmol) and sodium triacetoxyhydroborate (42 mg, 0.2 mmol). The reaction mixture was stirred at 50° C. overnight. The reaction mixture was quenched by the addition of saturated aqueous K₂CO₃. The aqueous layer was extracted with EtOAc (3×20 mL). The organic layer was dried over Na₂SO₄ and concentrated. The crude product was purified by HPLC (Mobile phase A: water with 0.01 M ammonia solution; Mobile phase B: acetonitrile; column temperature: 40° C., Gradient: 38-68% B over 10 min) to give N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide Isomer B (2.1 mg, 20.6% yield) as a white solid. ¹H NMR (400 MHz, Methanol-d₄) δ 7.71-7.69 (m, 1H), 7.58-7.56 (m, 1H), 7.12-7.05 (m, 2H), 6.27 (s, 1H), 4.52 (s, 2H), 4.25-4.21 (m, 2H), 3.93 (s, 3H), 2.79-2.70 (m, 2H), 2.59 (s, 3H), 2.44 (br. s, 1H), 2.33 (s, 3H), 2.30 (s, 3H), 2.25-2.22 (m, 1H), 2.10-2.04 (m, 1H), 1.63 (d, J=8 Hz, 3H), 1.41-1.32 (m, 1H), 1.16-1.13 (m, 1H), 0.82-0.79 (m, 1H); LCMS (M+H)⁺ calcd. 519.6; found 519.2.

### Example 19

The following compounds were synthesized according to the reduction conditions outlined in Examples 17 and 18 above utilizing diastereomeric mixtures of N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide and the appropriate aldehyde.

| Cmp. No. | Structure/Chemical Name | Characterization |
|---|---|---|
| 9 and 10 | 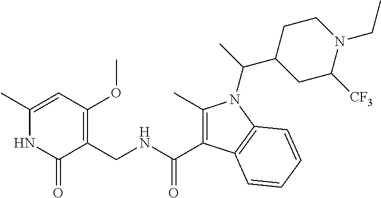 [SMILES: CCN1CCC(C(C)n2c(C)c(C(=O)NCc3c(OC)cc(C)[nH]c3=O)c3ccccc32)CC1C(F)(F)F] 1-(1-(1-ethyl-2- (trifluoromethyl)piperidin-4- yl)ethyl)-N-((4-methoxy-6- methyl-2-oxo-1,2-dihydropyridin- 3-yl)methyl)-2-methyl-1H-indole- 3-carboxamide Isomer A (9) and Isomer B (10) | Isomer A: ¹H NMR (400 MHz, Methanol-d₄) δ 7.72-7.70 (m, 1H), 7.59-7.57 (m, 1H), 7.14-7.05 (m, 2H), 6.29 (s, 1H), 4.52 (s, 2H), 4.23-4.18 (m, 1H), 3.95 (s, 3H), 3.10-3.07 (m, 1H), 2.87-2.80 (m, 2H), 2.70-2.63 (m, 1H), 2.58 (s, 3H), 2.53-2.47 (m, 2H), 2.30 (s, 3H), 2.02-1.99 (m, 1H), 1.60-1.59 (d, J = 8 Hz, 3H), 1.41-1.37 (m, 1H), 1.11- 1.01 (m, 5H); LCMS (M + H)⁺ calcd. 533.6; found 533.3. Isomer B: ¹H NMR (400 MHz, Methanol-d₄) δ 7.71-7.69 (m, 1H), 7.59-7.57 (m, 1H), 7.12-7.04 (m, 1H), 6.30 (s, 1H), 4.52 (s, 2H), 4.22-4.18 (m, 1H), 3.95 (s, 3H), 3.13 (m, 1H), 2.81- 2.64 (m, 3H), 2.57 (s, 3H), 2.31-2.25 (m, 1H), 2.22 (s, 3H), 2.18-2.15 (m, 2H), 1.62-1.61 (d, J = 7.2 Hz, 3H), 1.37- 1.28 (m, 3H), 1.07-1.04 (m, 1H), 1.00- |
| | | 0.96 (m, 3H), 0.80 (m, 1H); LCMS |
| | | (M + H)⁺ calcd. 533.6; found 533.3 |
| 38 and 39 | 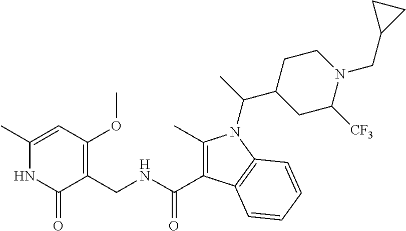 [SMILES: COc1cc(C)[nH]c(=O)c1CNC(=O)c1c(C)n(C(C)C2CCN(CC3CC3)C(C(F)(F)F)C2)c2ccccc12] 1-(1-(1-(cyclopropylmethyl)-2- (trifluoromethyl)piperidin-4- yl)ethyl)-N-((4-methoxy-6- methyl-2-oxo-1,2-dihydropyridin- 3-yl)methyl)-2-methyl-1H-indole- 3-carboxamide Isomer A (38) and Isomer B (39) | Isomer A: ¹H NMR (400 MHz, Methanol-d₄) δ 7.63-7.61 (m, 1H), 7.50-7.48 (m, 1H), 7.05-6.97 (m, 2H), 6.21 (s, 1H), 4.43 (s, 2H), 4.15-4.11 (m, 1H), 3.86 (s, 3H), 3.31-3.29 (m, 1H), 2.76 (m, 1H), 2.62-2.36 (m, 7H), 2.22 (s, 3H), 1.93 (m, 1H), 1.52-1.50 (d, J = 6.8 Hz, 3H), 1.36-1.31 (m, 1H), 1.19 (s, 1H), 1.02-1.00 (m, 1H), 0.81-0.78 (m, 1H), 0.44-0.42 (m, 2H) 0.09-0.02 (m, 2H); LCMS (M + H)⁺ calcd. calcd 559.7; found 559.3. Isomer B: ¹H NMR (400 MHz, Methanol-d₄) δ 7.68-7.66 (m, 1H), 7.56-7.54 (m, 1H), 7.10-7.01 (m, 2H), 6.26 (s, 1H), 4.49 (s, 2H), 4.20-4.15 (m, 1H), 3.92 (s, 3H), 3.12 (m, 1H), 3.04- 3.01 (m, 1H), 2.55 (s, 3H), 2.48-2.46 (m, 3H), 2.27 (s, 3H), 2.21-2.14 (m, 2H), 1.59-1.1.58 (d, J = 6.8 Hz, 3H), 1.38-1.32 (m, 1H), 1.09-1.03 (m, 1H), 0.78-0.74 (m, 2H), 0.44-0.42 (m, 2H), |
| | | 0.07-0.02 (m, 2H); LCMS (M + H)⁺ |
| | | calcd. calcd 559.7; found 559.3. |

### Example 20: Separation of enantiomers of N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer A, compound 12 to afford Enantiomer 1a (23) and Enantiomer 1b (22)

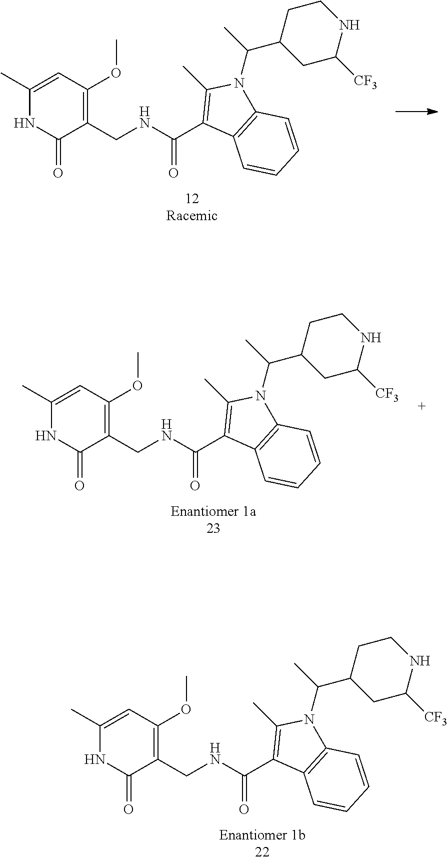 [SMILES: COc1cc(C)[nH]c(=O)c1CNC(=O)c1c(C)n(C(C)C2CCNC(C(F)(F)F)C2)c2ccccc12 ; COc1cc(C)[nH]c(=O)c1CNC(=O)c1c(C)n(C(C)C2CCNC(C(F)(F)F)C2)c2ccccc12 ; COc1cc(C)[nH]c(=O)c1CNC(=O)c1c(C)n(C(C)C2CCNC(C(F)(F)F)C2)c2ccccc12]

N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer A, compound 12 (1.10 g, 2.18 mmol) was purified by chiral SFC (Chiralpak AD-H) to give product Enantiomer 1a, compound 23 (497 mg, 45.4% yield) and Enantiomer 1b, compound 22 (477 mg, 43.6% yield) as a white solid.

Enantiomer 1a: ¹H NMR (400 MHz, Methanol-d₄) δ 7.72-7.70 (m, 1H), 7.58-7.56 (m, 1H), 7.14-7.07 (m, 2H), 6.26 (s, 1H), 4.52 (s, 2H), 4.24-4.19 (m, 1H), 3.93 (s, 3H), 3.21-3.18 (m, 1H), 2.97 (brs, 1H), 2.74-2.68 (m, 2H), 2.59 (s, 3H), 2.50-2.48 (m, 1H), 2.31 (s, 3H), 2.04-2.02 (m, 1H), 1.61 (d, J=8 Hz, 3H), 1.32-1.27 (m, 1H), 1.08-0.91 (m, 2H); LCMS (M+H)⁺ calcd. 505.6; found 505.1.

Enantiomer 1b: ¹H NMR (400 MHz, Methanol-d₄) δ 7.72-7.70 (m, 1H), 7.58-7.56 (m, 1H), 7.14-7.07 (m, 2H), 6.26 (s, 1H), 4.52 (s, 2H), 4.27-4.22 (m, 1H), 3.95 (s, 3H), 3.21-3.18 (m, 1H), 2.93-2.90 (m, 1H), 2.97 (brs, 1H), 2.7368 (m, 2H), 2.59 (s, 3H), 2.50-2.48 (m, 1H), 2.31 (s, 3H), 2.04-2.02 (m, 1H), 1.62 (d, J=8 Hz, 3H), 1.32-1.23 (m, 1H), 1.08-0.94 (m, 2H); LCMS (M+H)⁺ calcd. 505.6; found 505.1.

### Example 21: Separation of enantiomers of N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer A, compound 14 to afford Enantiomer 2a (18) and Enantiomer 2b (20)

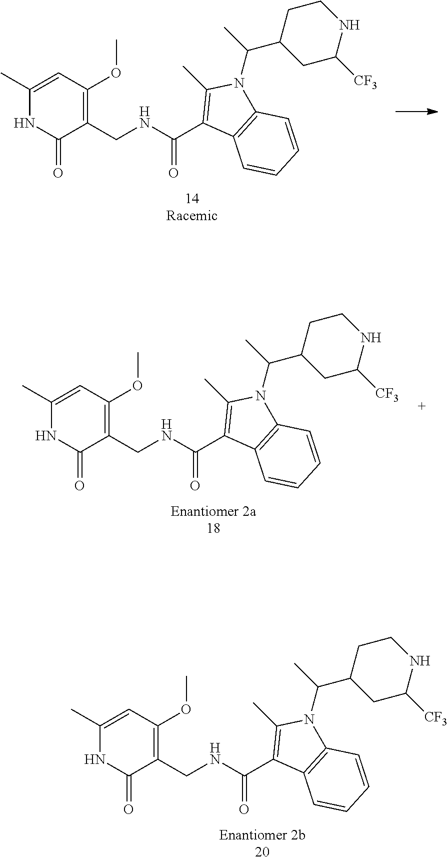 [SMILES: COc1cc(C)[nH]c(=O)c1CNC(=O)c1c(C)n(C(C)C2CCNC(C(F)(F)F)C2)c2ccccc12 ; COc1cc(C)[nH]c(=O)c1CNC(=O)c1c(C)n(C(C)C2CCNC(C(F)(F)F)C2)c2ccccc12 ; COc1cc(C)[nH]c(=O)c1CNC(=O)c1c(C)n(C(C)C2CCNC(C(F)(F)F)C2)c2ccccc12]

N-((4-methoxy-6-methyl-2-oxo-1,2-dihydropyridin-3-yl)methyl)-2-methyl-1-(1-(2-(trifluoromethyl)piperidin-4-yl)ethyl)-1H-indole-3-carboxamide Isomer A, compound 14 (780 mg, 1.55 mmol) was purified by chiral SFC separation to give product Enantiomer 2a, compound 18 (260 mg, 33.3% yield) and Enantiomer 2a, compound 20 (270 mg, 34.6% yield) as a white solid.

Enantiomer 2a: ¹H NMR (400 MHz, Methanol-d₄) δ 7.73-7.71 (m, 1H), 7.60-7.59 (m, 1H), 7.14-7.07 (m, 2H), 6.28 (s, 1H), 4.53 (s, 2H), 4.27-4.22 (m, 1H), 3.95 (s, 3H), 2.91 (d, J=12 Hz, 1H), 2.64-2.61 (m, 1H and s, 3H), 2.53-2.50 (m, 1H), 2.43-2.39 (m, 1H), 2.33 (s, 3H), 2.23-2.19 (m, 1H), 1.64 (d, J=8 Hz, 3H), 1.31-1.25 (m, 1H), 1.03-0.99 (m, 1H), 0.84-0.81 (m, 1H); LCMS (M+H)⁺ calcd. 505.6; found 505.1.

Enantiomer 2b: ¹H NMR (400 MHz, Methanol-d₄) δ 7.73-7.71 (m, 1H), 7.60-7.59 (m, 1H), 7.14-7.07 (m, 2H), 6.28 (s, 1H), 4.53 (s, 2H), 4.27-4.22 (m, 1H), 3.95 (s, 3H), 2.91 (d, J=12 Hz, 1H), 2.64-2.61 (m, 1H and s, 3H), 2.53-2.50 (m, 1H), 2.43-2.39 (m, 1H), 2.33 (s, 3H), 2.23-2.19 (m, 1H), 1.64 (d, J=8 Hz, 3H), 1.31-1.25 (m, 1H), 1.02-0.98 (m, 1H), 0.84-0.81 (m, 1H); LCMS (M+H)⁺ calcd. 505.6; found 505.1.

### Example 22: Separation of enantiomers of N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide (24 and 21)

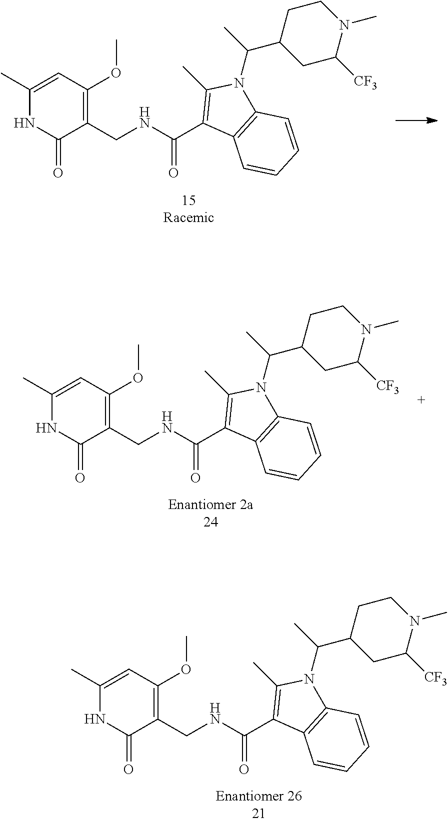

N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide Isomer B, compound 15 (370 mg, 0.71 mmol) was separated by chiral SFC (Column: Chiralpak AD 250 mm*50 mm, 10 um; Mobile phase: Supercritical CO₂/Base-MeOH=60/40; Flow rate: 200 ml/min; Wavelength: 220 nm) to give product Enantiomer 2a, compound 24 (150 mg, 40.5% yield) and Enantiomer 2b, compound 21 (160 mg, 43.2% yield) as a white solid.

Enantiomer 2a: Chiral SFC retention time: 9.4 min (Column: AD-H, mobile phase A: supercritical CO₂ mobile phase B: gradient 5 to 40% iPrOH with 0.05% diethylamine). ¹H NMR (400 MHz, Methanol-d₄) δ 7.69 (m, 1H), 7.58-7.56 (m, 1H), 7.12-7.05 (m, 2H), 6.26 (s, 1H), 4.51 (s, 2H), 4.25-4.19 (m, 1H), 3.93 (s, 3H), 2.80-2.70 (m, 1H and m, 1H), 2.59 (s, 3H), 2.44-2.39 (m, 1H), 2.34 (m, 3H), 2.31 (s, 3H), 2.25-2.22 (m, 1H), 2.10-2.04 (m, 1H), 1.62 (d, J=4 Hz, 3H), 1.38-1.34 (m, 1H), 1.18-1.15 (m, 1H), 0.82-0.80 (m, 1H); LCMS (M+H)⁺ calcd. 519.6; found 519.3.

Enantiomer 2b: Chiral SFC retention time: 10.5 min (Column: AD-H, mobile phase A: supercritical CO₂ mobile phase B: gradient 5 to 40% iPrOH with 0.05% diethylamine). ¹H NMR (400 MHz, Methanol-d₄) δ 7.71-7.69 (m, 1H), 7.58-7.56 (d, J=8 Hz, 1H), 7.12-7.05 (m, 2H), 6.26 (s, 1H), 4.51 (s, 2H), 4.25-4.19 (m, 1H), 3.93 (s, 3H), 2.81-2.70 (m, 1H and m, 1H), 2.60 (s, 3H), 2.48-2.44 (m, 1H), 2.33 (s, 3H), 2.31 (s, 3H), 2.25-2.22 (m, 1H), 2.07-2.04 (m, 1H), 1.62 (d, J=4 Hz, 3H), 1.39-1.34 (m, 1H), 1.12-1.09 (m, 1H), 0.82-0.80 (m, 1H); LCMS (M+H)⁺ calcd. 519.6; found 519.3.

### Example 23: Synthesis and separation of enantiomers of N-((4,6-dimethyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide (26 and 25)

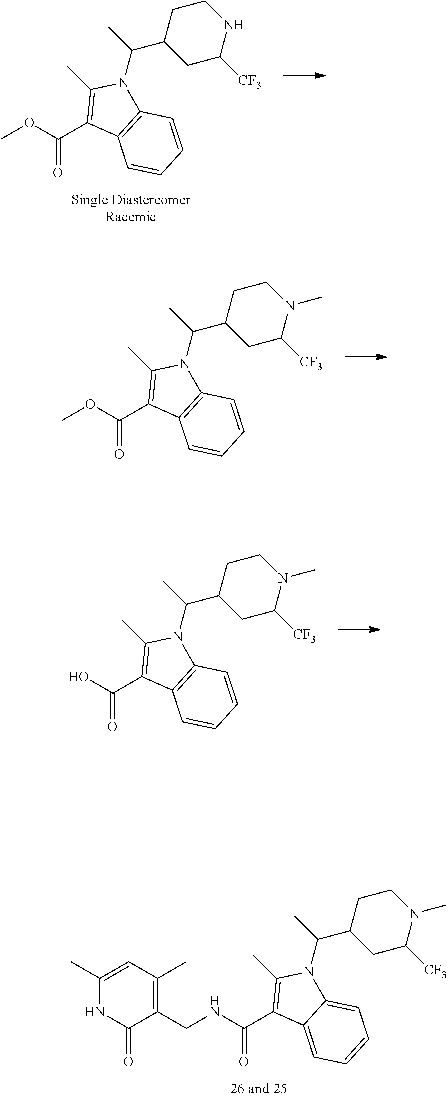

### Step 1: Synthesis of methyl 2-methyl-1-(1-(2-(trifluoromethyl)piperidin-4-yl)ethyl)-1H-indole-3-carboxylate

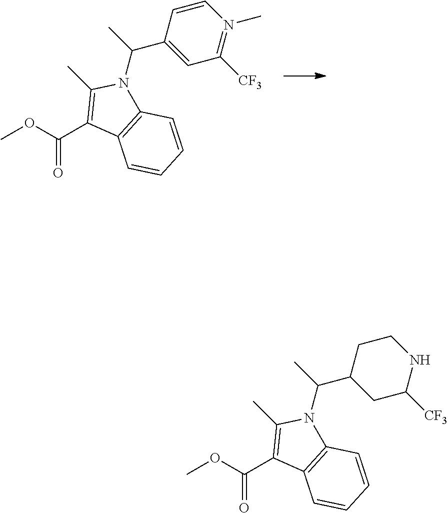

Methyl 2-methyl-1-(1-(2-(trifluoromethyl)piperidin-4-yl)ethyl)-1H-indole-3-carboxylate was synthesized using the same protocol described for methyl 2-methyl-1-(1-(2-(trifluoromethyl)piperidin-4-yl)ethyl)-1H-indole-3-carboxylate (see below).

### Synthesis of methyl 2-methyl-1-(1-(1-methyl-2-(trifluoromethyl)piperidin-4-yl)ethyl)-1H-indole-3-carboxylate

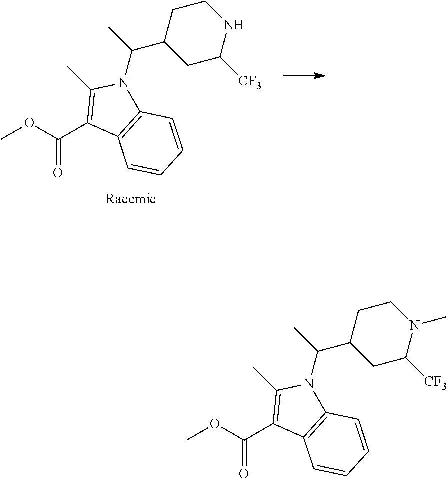

To a solution of methyl 2-methyl-1-(1-(2-(trifluoromethyl)piperidin-4-yl)ethyl)-1H-indole-3-carboxylate (300.0 mg, 0.814 mmol) in 1,2-dichloroethane (2 mL) was added sodium triacetoxyhydroborate (690.4 mg, 3.26 mmol) and paraformaldehyde (293.4 mg, 3.26 mmol) and acetic acid (0.05 mL) at ambient temperature. The mixture was stirred at 70° C. for 3 h. After cooling to room temperature, the mixture was quenched by the addition of MeOH.

The resulting mixture was filtered and the filtrate was concentrated in vacuo. The residue was purified by silica gel chromatography (petroleum ether:EtOAc=5:1) to afford methyl 2-methyl-1-(1-(1-methyl-2-(trifluoromethyl)-4-piperidyl)ethyl)indole-3-carboxylate (280.0 mg, 0.732 mmol, 89.9% yield) as a light yellow oil.

### Synthesis of 2-methyl-1-(1-(1-methyl-2-(trifluoromethyl)piperidin-4-yl)ethyl)-1H-indole-3-carboxylic Acid

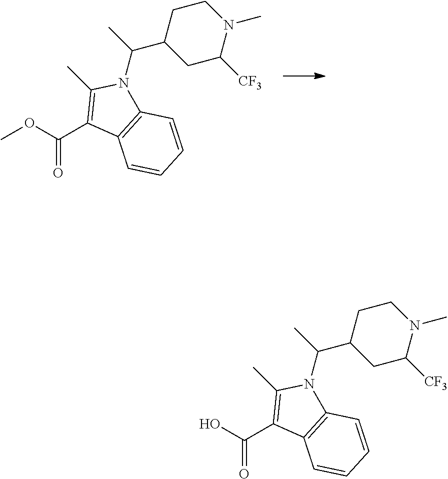

To a solution of methyl 2-methyl-1-(1-(1-methyl-2-(trifluoromethyl)piperidin-4-yl)ethyl)-1H-indole-3-carboxylate (280.0 mg, 0.732 mmol) in MeOH (2.0 mL) was added a solution of NaOH (0.977 mL, 3 M) at ambient temperature. The mixture was stirred at 70° C. for 8 h. The mixture was acidified to pH 5-6 using aqueous 1M HCl at 0° C. The reaction mixture was concentrated in vacuo to afford the residue. To the residue was added CH₂Cl₂ (3 mL) and the resultant mixture was filtered. The filtrate was concentrated in vacuo to afford 2-methyl-1-(1-(1-methyl-2-(trifluoromethyl)piperidin-4-yl)ethyl)-1H-indole-3-carboxylic acid (240.0 mg) as a yellow oil.

### Synthesis separation of enantiomers of N-((4,6-dimethyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide

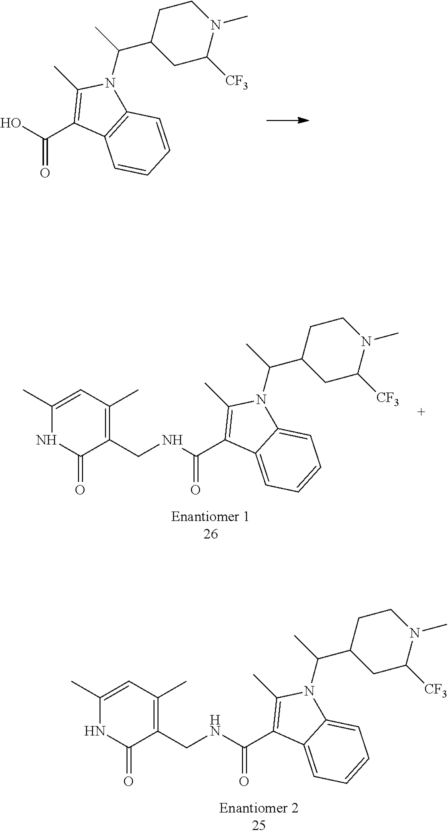

To a solution of 2-methyl-1-(1-(1-methyl-2-(trifluoromethyl)piperidin-4-yl)ethyl)-1H-indole-3-carboxylic acid (200.0 mg, 0.543 mmol) in CH₂Cl₂ (2 mL) was added COMU (465.0 mg, 1.09 mmol) and DIEA (350.8 mg, 2.71 mmol) at ambient temperature. The resulting solution was stirred at 20° C. for 10 min. Then 3-(aminomethyl)-4,6-dimethyl-1H-pyridin-2-one hydrochloride (153.6 mg, 0.814 mmol) was added and allowed to stir for another 3 h. The reaction mixture was purified by prep-HPLC (HPLC separation conditions: Column: Gemini 150*25 mm*5 um, Mobile phase A: water with 0.05% ammonia solution, Mobile phase B: acetonitrile, Column temperature: 30° C., Gradient: 31˜51% B 10 min, Flow rate: 25 mL/min) and then subjected to SFC (AD (250 mm*30 mm, 10 um), condition: 50% IPA+ammonia) to afford Enantiomer 1 of N-((4,6-dimethyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide, compound 26 (12.0 mg, 23.9 μmol, 4.4% yield) as a white solid and Enantiomer 2 of N-((4,6-dimethyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide, compound 25 (4.50 mg, 8.95 μma 1.65% yield) as a light red solid.

Enantiomer 1: ¹H NMR (400 MHz, DMSO-d₆) δ.7.88-7.68 (m, 2H), 7.65-7.55 (m, 1H), 7.12-7.00 (m, 2H), 5.86 (s, 1H), 4.37-4.17 (m, 3H), 2.85-2.75 (m, 1H), 2.70-2.62 (m, 2H), 2.58 (s, 2H), 2.44-2.31 (m, 2H), 2.28-2.19 (m, 5H), 2.15-2.04 (m, 4H), 2.00-1.9. (m, 1H), 1.60-1.47 (m, 3H), 1.42-1.29 (m, 1H), 1.16-1.0 (m, 2H), 0.66-0.56 (m, 1H); LCMS (M+H)⁺ m/z: calcd. 503.6, found 503.1.

Enantiomer 2: ¹H NMR (400 MHz, DMSO-d₆): δ.7.80-7.67 (m, 2H), 7.62-7.56 (m, 1H), 7.09-6.99 (m, 2H), 5.85 (s, 1H), 4.33-4.18 (m, 3H), 2.84-2.74 (m, 1H), 2.69-2.60 (m, 2H), 2.56 (s, 2H), 2.37-2.30 (m, 1H), 2.26-2.18 (m, 5H), 2.13-2.02 (m, 4H), 1.99-1.89 (m, 1H), 1.58-1.44 (m, 4H), 1.39-1.28 (m, 1H), 1.12-0.99 (m, 2H), 0.64-0.55 (m, 1H); LCMS (M+H)⁺ m/z: calcd. 503.6, found 503.1.

### Example 24: Synthesis of Single Enantiomer of N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide (21)

The synthesis below affords enantiomer 2b of example 22, compound 21, although absolute stereochemistry is not defined.

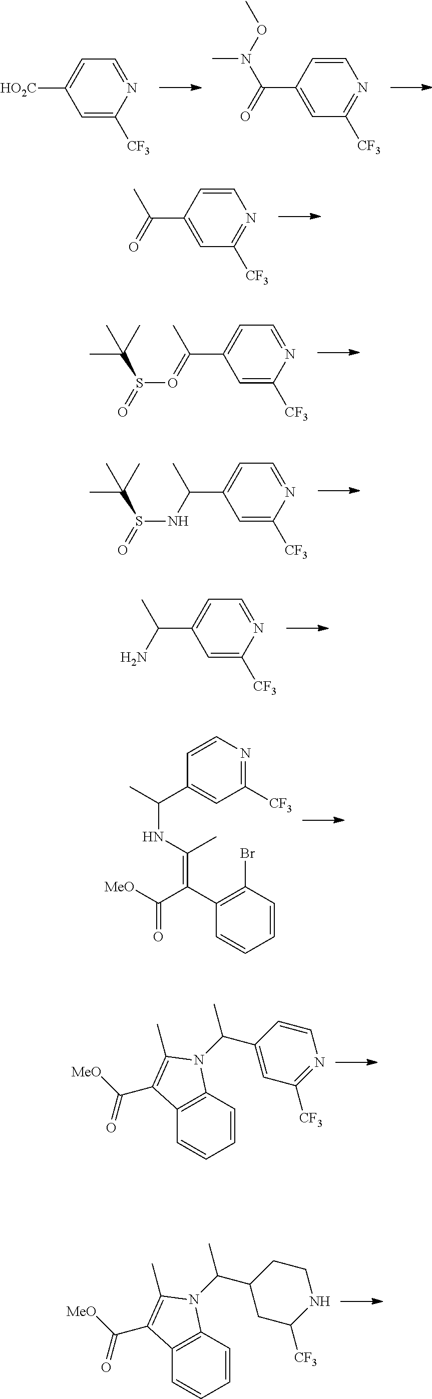

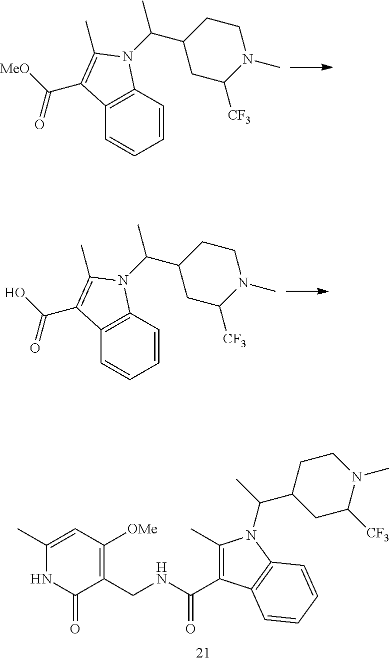

### Step 1: Synthesis of (R)-2-methyl-N-(1-(2-(trifluoromethyl)pyridin-4-yl)ethyl)propane-2-sulfinamide

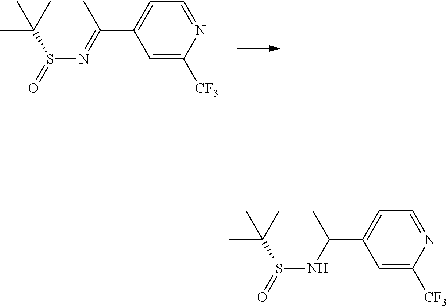 [SMILES: C/C(=N\[S@](=O)C(C)(C)C)c1ccnc(C(F)(F)F)c1 ; CC(N[S@](=O)C(C)(C)C)c1ccnc(C(F)(F)F)c1]

To a solution of (R)-2-methyl-N-(1-(2-(trifluoromethyl)pyridin-4-yl)ethylidene)propane-2-sulfinamide (10.0 g, 34.2 mmol) in THF (150 mL) was added in a dropwise manner a solution of DIBAL-H (75 mL, 1 M in toluene) at −70° C. under N₂ (g). The mixture was stirred at −70° C. for 2 h. The mixture was quenched with MeOH at −70° C. and a solution of NaOH (150 mL, 2M) was added at 0° C. The mixture was extracted with EtOAc (3×200 mL). The combined organic layers were dried over anhydrous Na₂SO₄ and concentrated in vacuo. The crude product was purified by silica gel chromatography (petroleum ether/EtOAc=10:1 to 1:1) to give (R)-2-methyl-N-(1-(2-(trifluoromethyl)pyridin-4-yl)ethyl)propane-2-sulfinamide (7.50 g, 25.5 mmol, 75.9% yield, >98% de) as a yellow oil. Sixteen additional batches of (R)-2-methyl-N-(1-(2-(trifluoromethyl)pyridin-4-yl)ethyl)propane-2-sulfinamide were synthesized in parallel on similar scale using the procedure outlined above.

### Step 2: Synthesis of 1-(2-(trifluoromethyl)pyridin-4-yl)ethan-1-amine

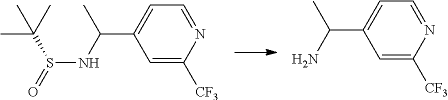 [SMILES: CC(N[S@](=O)C(C)(C)C)c1ccnc(C(F)(F)F)c1 ; CC(N)c1ccnc(C(F)(F)F)c1]

To a solution of (R)-2-methyl-N-(1-(2-(trifluoromethyl)pyridin-4-yl)ethyl)propane-2-sulfinamide (10.0 g, 34.0 mmol) in MeOH (20 mL) was added a solution of HCl (10.0 mL, 40.0 mmol, 4M in MeOH) portion wise at 0° C. The mixture was stirred at 25° C. for 2 h. The mixture was concentrated in vacuo and water (25 mL) was subsequently added. The mixture was extracted with EtOAc (2×30 mL). The aqueous layer was cooled to 0° C. and basified to pH ˜9 with 1 M NaOH solution. The resulting basic aqueous solution extracted with CH₂Cl₂ (3×30 mL). The combined organic layers were dried over anhydrous Na₂SO₄ and concentrated in vacuo to give 1-(2-(trifluoromethyl)pyridin-4-yl)ethanamine (5.83 g), which was used directly in the subsequent step without further purification. Twelve additional batches of 1-(2-(trifluoromethyl)pyridin-4-yl)ethanamine were synthesized in parallel on similar scale using the procedure outlined above.

### Step 3: Synthesis of methyl 2-(2-bromophenyl)-3-((1-(2-(trifluoromethyl)pyridin-4-yl)ethyl)amino)but-2-enoate

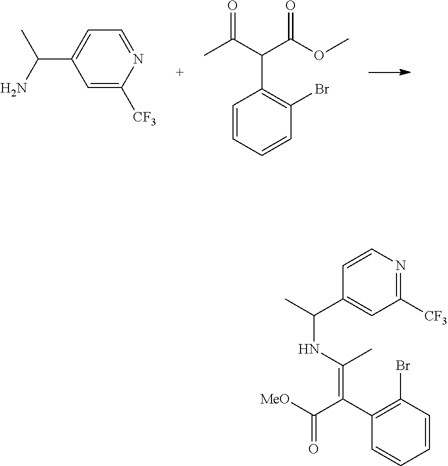 [SMILES: CC(N)c1ccnc(C(F)(F)F)c1 ; COC(=O)C(C(C)=O)c1ccccc1Br ; COC(=O)/C(=C(/C)NC(C)c1ccnc(C(F)(F)F)c1)c1ccccc1Br]

To a mixture of 1-(2-(trifluoromethyl)pyridin-4-yl)ethanamine (12.0 g, 63.1 mmol) and methyl 2-(2-bromophenyl)-3-oxobutanoate (15.4 g, 56.8 mmol) in EtOH (50 mL) was added AcOH (5.68 g, 94.65 mmol) in one portion. The mixture was stirred at 80° C. for 15 h. The mixture was concentrated in vacuo to give crude product. The crude product was purified by silica gel chromatography (petroleum ether/EtOAc=20:1 to 10:1) to give methyl 2-(2-bromophenyl)-3-((1-(2-(trifluoromethyl)pyridin-4-yl)ethyl)amino)but-2-enoate (10.83 g, 48.9 mmol, 79.7% yield) as a yellow oil. Six additional batches of methyl 2-(2-bromophenyl)-3-((1-(2-(trifluoromethyl)pyridin-4-yl)ethyl)amino)but-2-enoate were synthesized in parallel on similar scale using the procedure outlined above.

### Step 4: Synthesis of methyl 2-methyl-1-(1-(2-(trifluoromethyl)pyridin-4-yl)ethyl)-1H-indole-3-carboxylate

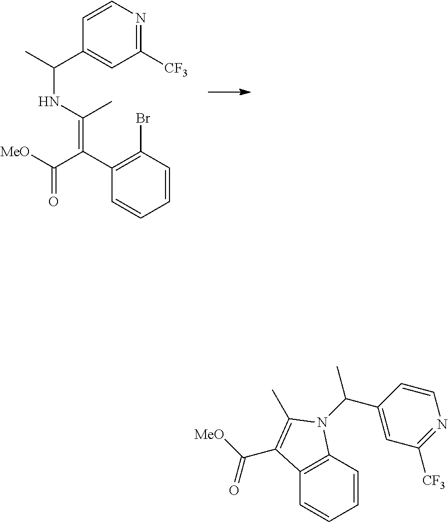 [SMILES: COC(=O)/C(=C(/C)NC(C)c1ccnc(C(F)(F)F)c1)c1ccccc1Br ; COC(=O)c1c(C)n(C(C)c2ccnc(C(F)(F)F)c2)c2ccccc12]

To a mixture of methyl 2-(2-bromophenyl)-3-((1-(2-(trifluoromethyl)pyridin-4-yl)ethyl)amino)but-2-enoate (7.00 g, 15.79 mmol) in 1,4-dioxane (150 mL) were added Pd₂(dba)₃ (1.45 g, 1.58 mmol), AcONa (3.89 g, 47.37 mmol) and tri-tert-butylphosphonium tetrafluoroborate (595.63 mg, 2.05 mmol) in one portion under N₂ (g). The mixture was stirred at 110° C. under N₂ (g) for 15 h. The mixture was filtered and the filtrate was concentrated under vacuum to give crude product. The crude product was purified by silica gel chromatography (petroleum ether/EtOAc=30:1 to 10:1) to give methyl 2-methyl-1-(1-(2-(trifluoromethyl)pyridin-4-yl)ethyl)-1H-indole-3-carboxylate (5.00 g, 13.8 mmol, 84.7% yield, 99% ee) as a yellow oil. Eighteen additional batches of methyl 2-methyl-1-(1-(2-(trifluoromethyl)pyridin-4-yl)ethyl)-1H-indole-3-carboxylate were synthesized in parallel on similar scale using the procedure outlined above. LCMS (M+H)⁺ m/z: calcd. 363.1: found 363.0.

### Step 5: Synthesis of methyl 2-methyl-1-(1-(2-(trifluoromethyl)piperidin-4-yl)ethyl)-1H-indole-3-carboxylate

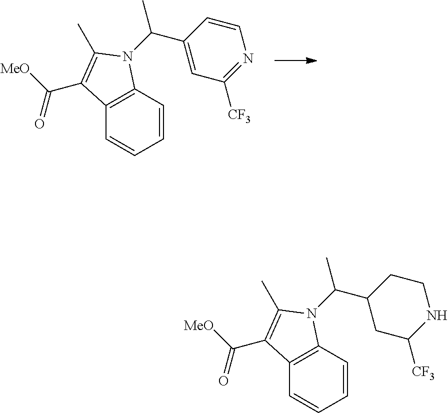 [SMILES: COC(=O)c1c(C)n(C(C)c2ccnc(C(F)(F)F)c2)c2ccccc12 ; COC(=O)c1c(C)n(C(C)C2CCNC(C(F)(F)F)C2)c2ccccc12]

To a mixture of methyl 2-methyl-1-(1-(2-(trifluoromethyl)pyridin-4-yl)ethyl)-1H-indole-3-carboxylate (5.00 g, 13.80 mmol) in MeOH (50 mL) were added PtO₂ (1.88 g, 8.28 mmol,) and HCl (12 M, 3.45 mL) in one portion at 25° C. The mixture was stirred at 25° C. for 15 h under H₂ (g) (15 psi). The mixture was filtered and the filtrate was concentrated under vacuum to give crude product as a mixture of diastereomers. The diastereomeric mixtures were purified by silica gel chromatography (petroleum ether/EtOAc=15:1 to 5:1) to give the desired diastereomer of methyl 2-methyl-1-(1-(2-(trifluoromethyl)piperidin-4-yl)ethyl)-1H-indole-3-carboxylate (2.22 g, 6.03 mmol, 43.7% yield, 91% ee) as a colorless oil. Eighteen additional batches of methyl 2-methyl-1-(1-(2-(trifluoromethyl)piperidin-4-yl)ethyl)-1H-indole-3-carboxylate were synthesized in parallel on similar scale using the procedure outlined above. LCMS (M+H)⁺ m/z: calcd. 369.1: found 369.1.

### Step 6: Synthesis of methyl 2-methyl-1-(1-(1-methyl-2-(trifluoromethyl)piperidin-4-yl)ethyl)-1H-indole-3-carboxylate

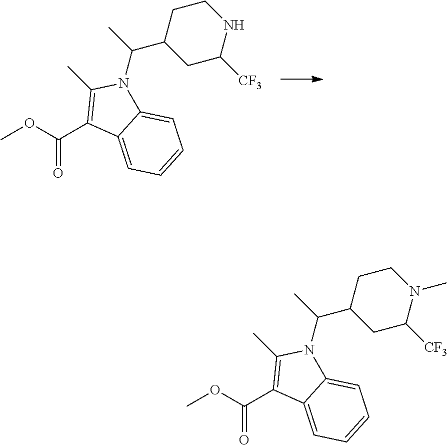

To a mixture of methyl 2-methyl-1-(1-(2-(trifluoromethyl)piperidin-4-yl)ethyl)-1H-indole-3-carboxylate (6.00 g, 16.29 mmol) in 1,2-dichloroethane (50.00 mL) was added paraformaldehyde (4.40 g, 48.87 mmol) in one portion at 25° C. The mixture was stirred at 75° C. for 2 h. The mixture was cooled to 25° C. followed by the portion wise addition of NaBH(OAc)₃ (10.36 g, 48.87 mmol). The mixture was subsequently heated to 75° C. for 15 h. The mixture was quenched with water (30 mL) and extracted with EtOAc (3×40 mL). The combined organic layers were dried over anhydrous Na₂SO₄ and concentrated in vacuo. The crude product was purified by silica gel chromatography (petroleum ether/EtOAc=20:1 to 5:1) and then recrystallized (MeOH/EtOAc=1:15) to give methyl 2-methyl-1-(-1-(1-methyl-2-(trifluoromethyl)piperidin-4-yl)ethyl)-1H-indole-3-carboxylate (4.00 g, 10.46 mmol, 67.4% yield, 99% ee) as a white solid. Seven additional batches of methyl 2-methyl-1-(1-(1-methyl-2-(trifluoromethyl)piperidin-4-yl)ethyl)-1H-indole-3-carboxylate were synthesized in parallel on similar scale using the procedure outlined above. LCMS (M+H⁺) m/z: calcd. 383.3: found 383.1.

### Step 7: Synthesis of 2-methyl-1-(1-(1-methyl-2-(trifluoromethyl)piperidin-4-yl)ethyl)-1H-indole-3-carboxylic Acid

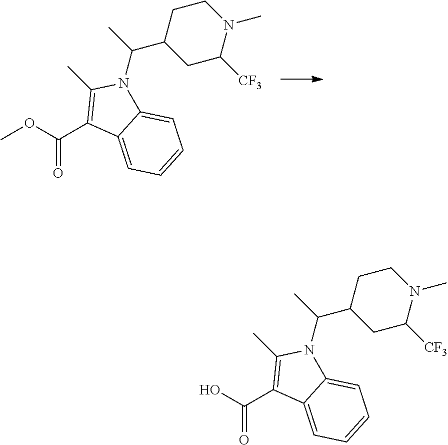

To a mixture of methyl 2-methyl-1-(1-(1-methyl-2-(trifluoromethyl)piperidin-4-yl)ethyl)-1H-indole-3-carboxylate (2.50 g, 6.54 mmol) in MeOH (50.00 mL) were added NaOH (1.31 g, 32.69 mmol) and H₂O (10.00 mL) in one portion at 25° C. The mixture was stirred at 85° C. for 30 h. The mixture was concentrated in vacuo. Water (20 mL) was added to the mixture and the mixture was acidified to pH 4 with aqueous 4N HCl solution at 0° C. The mixture was extracted with EtOAc (3×30 mL). The combined organic layers were dried over anhydrous Na₂SO₄ and concentrated under vacuum to give 2-methyl-1-(1-(1-methyl-2-(trifluoromethyl)piperidin-4-yl)ethyl)-1H-indole-3-carboxylic acid (2.27 g), which was used directly in the subsequent reaction without further purification. Eleven additional batches of 2-methyl-1-(1-(1-methyl-2-(trifluoromethyl)piperidin-4-yl)ethyl)-1H-indole-3-carboxylic acid were synthesized in parallel on similar scale using the procedure outlined above.

### Step 8: Synthesis of N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide

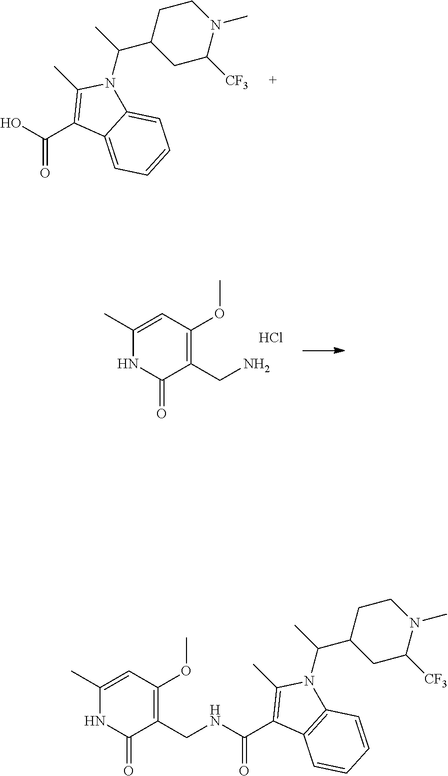

To a solution of 2-methyl-1-(1-(1-methyl-2-(trifluoromethyl)piperidin-4-yl)ethyl)-1H-indole-3-carboxylic acid (3.40 g, 9.23 mmol) in CH₂Cl₂ (100.00 mL) was sequentially added COMU (5.93 g, 13.84 mmol) and DIEA (4.17 g, 32.30 mmol). The mixture was stirred at 15° C. for 2 h, followed by cooling to 0° C. To the cooled solution was added 3-(aminomethyl)-4-methoxy-6-methylpyridin-2(1H)-one hydrochloride (2.83 g, 13.85 mmol) in one portion. The reaction mixture was stirred at 15° C. for 15 h. The mixture was quenched with water (100 mL) and extracted with CH₂Cl₂ (3×100 mL). The combined organic layers were dried over anhydrous Na₂SO₄ and concentrated under vacuum. The crude product was purified by silica gel chromatography (EtOAc/MeOH=50:1 to 20:1) to give N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide (3.44 g, 6.64 mmol, 68.5% yield) as an off-white solid. Seven additional batches of N-((4-methoxy-6-methyl-2-oxo-1,2-dihydropyridin-3-yl)methyl)-2-methyl-1-(1-(1-methyl-2-(trifluoromethyl)piperidin-4-yl)ethyl)-1H-indole-3-carboxamide (24.1 g total, 98.0% purity, 99% ee) were synthesized in parallel on similar scale using the procedure outlined above. Chiral SFC retention time: 6.7 min (Column: AD-H, mobile phase A: supercritical CO₂ mobile phase B: gradient 5 to 40% EtOH with 0.05% diethylamine). ¹H NMR (400 MHz, Methanol-d₄) δ 7.71 (d, J=7.5 Hz, 1H), 7.58 (d, J=7.9 Hz, 1H), 7.13-7.06 (m, 2H), 6.28 (s, 1H), 4.53 (s, 2H), 4.27-4.19 (m, 1H), 3.95 (s, 3H), 2.81-2.77 (m, 1H), 2.74-2.68 (m, 1H), 2.60 (s, 3H), 2.45-2.41 (m, 1H), 2.35 (s, 3H), 2.31 (s, 3H), 2.26-2.23 (m, 1H), 2.07 (t, J=11.7 Hz, 1H), 1.64 (d, J=7.0 Hz, 3H), 1.38-1.35 (m, 1H), 1.19-1.08 (m, 1H), 0.82-0.79 (m, 1H); LCMS (M+H)⁺ m/z: calcd. 519.2: found 519.1.

### Preparation: Synthesis of intermediate 2-methyl-1-(1-(2-(trifluoromethyl)piperidin-4-yl)ethyl)-1H-indole-3-carboxylic Acid

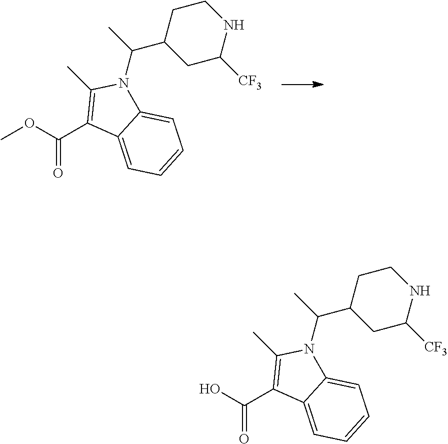 [SMILES: COC(=O)c1c(C)n(C(C)C2CCNC(C(F)(F)F)C2)c2ccccc12 ; Cc1c(C(=O)O)c2ccccc2n1C(C)C1CCNC(C(F)(F)F)C1]

To a stirred solution of methyl 2-methyl-1-(1-(2-(trifluoromethyl)piperidin-4-yl)ethyl)-1H-indole-3-carboxylate (200.0 mg, 0.543 mmol) in methanol (3 mL) and water (2 mL) was added NaOH (217.2 mg, 5.43 mmol). The mixture solution was stirred for 15 h at 85° C. The reaction solution was concentrated under vacuum, and the residue was adjusted to pH-7 by addition of aqueous 4N HCl. The solids was filtered out, and washed by petroleum ether (2×10 mL). The cake was dried to give 2-methyl-1-(1-(2-(trifluoromethyl)piperidin-4-yl)ethyl)-1H-indole-3-carboxylic acid (185.00 mg, crude) as off white solid. LCMS (M+H)⁺ calcd. 355.2; found 354.9.

### Example 25

The following compounds were synthesized using standard peptide coupling reagents, such as COMU or HATU, in the presence of appropriate pyridone ammonium chloride salt and common intermediate 2-methyl-1-(1-(2-(trifluoromethyl)piperidin-4-yl)ethyl)-1H-indole-3-carboxylic acid outlined directly above.

| Cmp. No. | Structure/Chemical Name | Characterization |
|---|---|---|
| 5 | 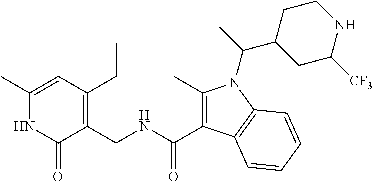 [SMILES: CCc1cc(C)[nH]c(=O)c1CNC(=O)c1c(C)n(C(C)C2CCNC(C(F)(F)F)C2)c2ccccc12] N-((4-ethyl-6-methyl-2-oxo-1,2- dihydropyridin-3-yl)methyl)-2- methyl-1-(1-(2- (trifluoromethyl)piperidin-4- yl)ethyl)-1H-indole-3- carboxamide | ¹H NMR (400 MHz, DMSO-d₆) δ 7.74 (d, J = 7.0 Hz, 1H), 7.62 (d, J = 7.5 Hz, 1H), 7.19-7.05 (m, 2H), 6.17 (s, 1H), 4.61-4.51 (m, 2H), 4.33-4.18 (m, 1H), 3.00-2.88 (m, 1H), 2.87- 2.71 (m, 3H), 2.70-2.59 (m, 3H), 2.53 (d, J = 11.0 Hz, 1H), 2.47-2.34 (m, 1H), 2.29 (s, 3H), 2.23 (d, J = 13.6 Hz, 1H), 1.66 (d, J = 7.0 Hz, 3H), 1.35- 1.20 (m, 4H), 1.08-0.92 (m, 1H), 0.85 (d, J = 12.0 Hz, 1H); LCMS (M + H)⁺ calcd. 503.3; found 503.2 |
| 6 | 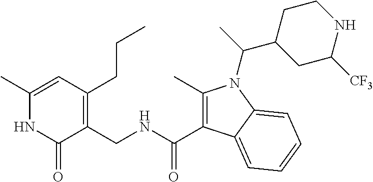 [SMILES: CCCc1cc(C)[nH]c(=O)c1CNC(=O)c1c(C)n(C(C)C2CCNC(C(F)(F)F)C2)c2ccccc12] 2-methyl-N-((6-methyl-2-oxo-4- propyl-1,2-dihydropyridin-3- yl)methyl)-1-(1-(2- (trifluoromethyl)piperidin-4- yl)ethyl)-1H-indole-3- carboxamide | ¹H NMR (400 MHz, DMSO-d₆) δ 11.56 (s, 1 H), 7.64 (d, J = 7.09 Hz, 1 H), 7.58- 7.48 (m, 2 H), 7.04-6.92 (m, 2 H), 5.83 (s, 1 H), 4.26 (s, 2 H), 4.17-4.07(m, 1 H), 3.16 (s, 1 H), 2.70 (d, J = 11.5 Hz, 1 H), 2.60 (s, 1 H), 2.42 (s, 5 H), 2.39-2.26 (m, 2 H), 2.19-2.10 (m, 1 H), 2.04 (s, 3 H), 1.95 (d, J = 11.49 Hz, 1 H), 1.46 (d, J = 6.85 Hz, 5 H), 1.16-1.05 (m, 1 H), 0.84 (t, J = 7.21 Hz, 3 H), 0.52 (d, J = 11.74 Hz, 1 H); LCMS (M + H)⁺ m/z: calcd. 517.27: found 517.0 |
| 7 | 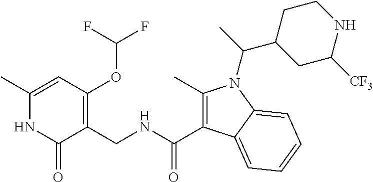 [SMILES: Cc1cc(OC(F)F)c(CNC(=O)c2c(C)n(C(C)C3CCNC(C(F)(F)F)C3)c3ccccc23)c(=O)[nH]1] N-((4-(difluoromethoxy)-6- methyl-2-oxo-1,2-dihydropyridin- 3-yl)methyl)-2-methyl-1-(1-(2- (trifluoromethyl)piperidin-4- yl)ethyl)-1H-indole-3- carboxamide | ¹H NMR (400 MHz, Methanol-d₄) δ 7.76 (d, J = 6.8 Hz, 1H), 7.60 (d, J = 7.6 Hz, 1H), 7.25-6.88 (m, 3H), 6.22 (s, 1H), 4.55 (s, 2H), 4.29-4.20 (m, 1H), 2.91 (d, J = 12.5 Hz, 1H), 2.74 (s, 3H), 2.52 (d, J = 11.0 Hz, 1H), 2.44-2.36 (m, 1H), 2.32 (s, 3H), 2.22 (d, J = 11.2 Hz, 1H), 1.65 (d, J = 6.8 Hz, 3H), 1.33-1.22 (m, 1H), 1.08- 0.94 (m, 2H), 0.84 (d, J = 13.0 Hz, 1H); LCMS (M + H)⁺ m/z: calcd 541.22; found 541.1 |

### Example 26

The following additional compounds in Table 1 below were prepared using the methods described herein and the appropriate starting materials and reagents.

TABLE 1

| Compound # | Structure |
|---|---|
| 44 | 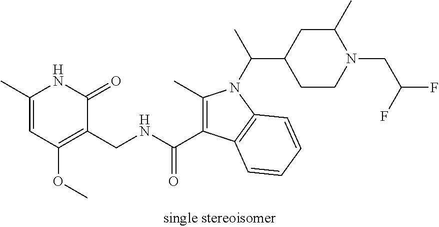 [SMILES: COc1cc(C)[nH]c(=O)c1CNC(=O)c1c(C)n(C(C)C2CCN(CC(F)F)C(C)C2)c2ccccc12] |
| 45 | 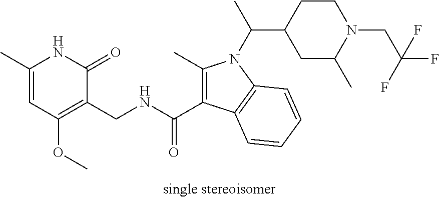 [SMILES: COc1cc(C)[nH]c(=O)c1CNC(=O)c1c(C)n(C(C)C2CCN(CC(F)(F)F)C(C)C2)c2ccccc12] |
| 46 | 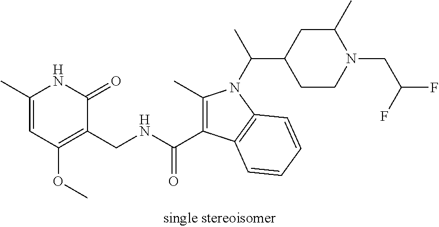 [SMILES: COc1cc(C)[nH]c(=O)c1CNC(=O)c1c(C)n(C(C)C2CCN(CC(F)F)C(C)C2)c2ccccc12] |
| 47 | 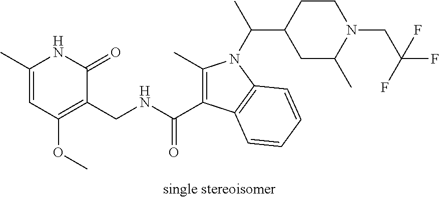 [SMILES: COc1cc(C)[nH]c(=O)c1CNC(=O)c1c(C)n(C(C)C2CCN(CC(F)(F)F)C(C)C2)c2ccccc12] |
| 48 | 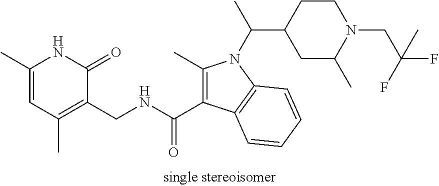 [SMILES: Cc1cc(C)c(CNC(=O)c2c(C)n(C(C)C3CCN(CC(C)(F)F)C(C)C3)c3ccccc23)c(=O)[nH]1] |
| 49 | 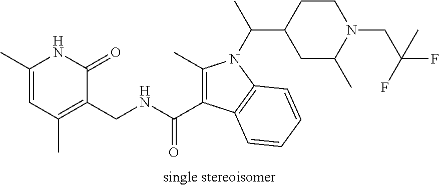 [SMILES: Cc1cc(C)c(CNC(=O)c2c(C)n(C(C)C3CCN(CC(C)(F)F)C(C)C3)c3ccccc23)c(=O)[nH]1] |
| 50 | 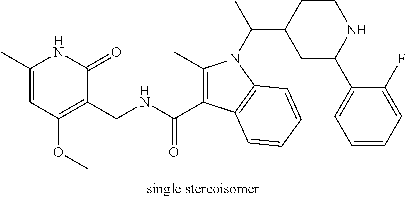 [SMILES: COc1cc(C)[nH]c(=O)c1CNC(=O)c1c(C)n(C(C)C2CCNC(c3ccccc3F)C2)c2ccccc12] |
| 51 | 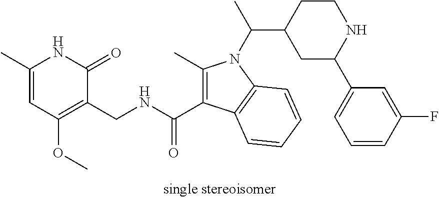 [SMILES: COc1cc(C)[nH]c(=O)c1CNC(=O)c1c(C)n(C(C)C2CCNC(c3cccc(F)c3)C2)c2ccccc12] |
| 52 | 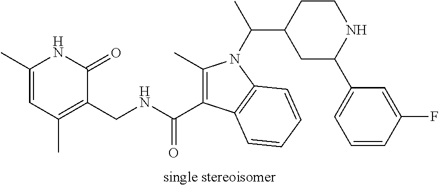 [SMILES: Cc1cc(C)c(CNC(=O)c2c(C)n(C(C)C3CCNC(c4cccc(F)c4)C3)c3ccccc23)c(=O)[nH]1] |
| 53 | 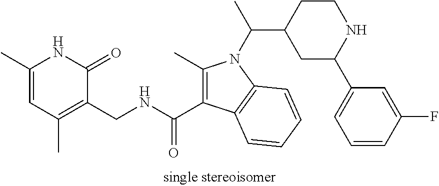 [SMILES: Cc1cc(C)c(CNC(=O)c2c(C)n(C(C)C3CCNC(c4cccc(F)c4)C3)c3ccccc23)c(=O)[nH]1] |
| 54 | 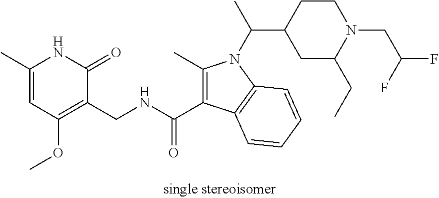 [SMILES: CCC1CC(C(C)n2c(C)c(C(=O)NCc3c(OC)cc(C)[nH]c3=O)c3ccccc32)CCN1CC(F)F] |
| 55 | 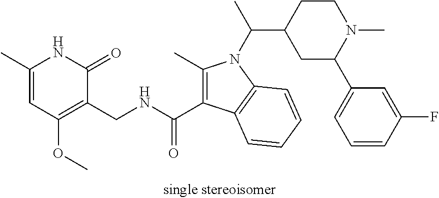 [SMILES: COc1cc(C)[nH]c(=O)c1CNC(=O)c1c(C)n(C(C)C2CCN(C)C(c3cccc(F)c3)C2)c2ccccc12] |
| 56 | 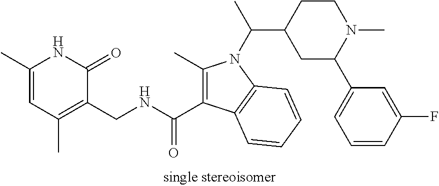 [SMILES: Cc1cc(C)c(CNC(=O)c2c(C)n(C(C)C3CCN(C)C(c4cccc(F)c4)C3)c3ccccc23)c(=O)[nH]1] |
| 57 | 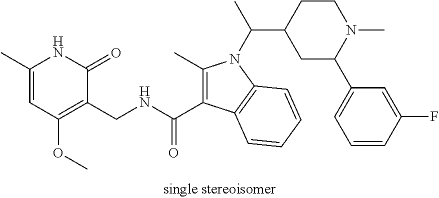 [SMILES: COc1cc(C)[nH]c(=O)c1CNC(=O)c1c(C)n(C(C)C2CCN(C)C(c3cccc(F)c3)C2)c2ccccc12] |
| 58 | 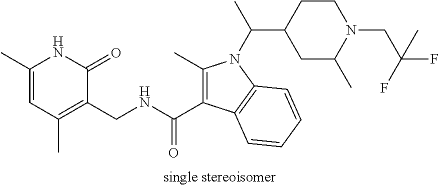 [SMILES: Cc1cc(C)c(CNC(=O)c2c(C)n(C(C)C3CCN(CC(C)(F)F)C(C)C3)c3ccccc23)c(=O)[nH]1] |
| 59 | 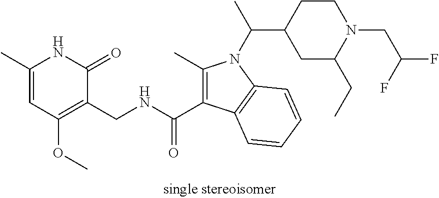 [SMILES: CCC1CC(C(C)n2c(C)c(C(=O)NCc3c(OC)cc(C)[nH]c3=O)c3ccccc32)CCN1CC(F)F] |
| 60 | 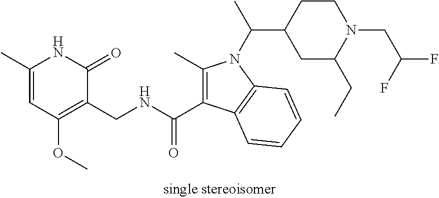 [SMILES: CCC1CC(C(C)n2c(C)c(C(=O)NCc3c(OC)cc(C)[nH]c3=O)c3ccccc32)CCN1CC(F)F] |
| 61 | 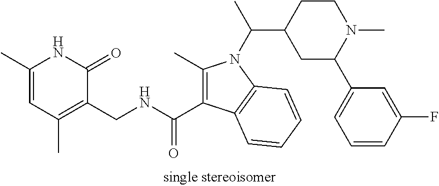 [SMILES: Cc1cc(C)c(CNC(=O)c2c(C)n(C(C)C3CCN(C)C(c4cccc(F)c4)C3)c3ccccc23)c(=O)[nH]1] |
| 62 | 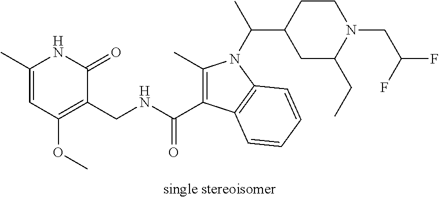 [SMILES: CCC1CC(C(C)n2c(C)c(C(=O)NCc3c(OC)cc(C)[nH]c3=O)c3ccccc32)CCN1CC(F)F] |
| 63 | 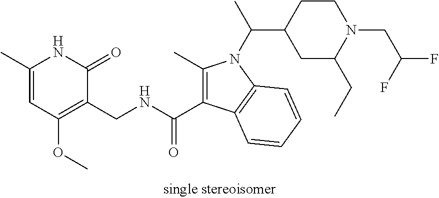 [SMILES: CCC1CC(C(C)n2c(C)c(C(=O)NCc3c(OC)cc(C)[nH]c3=O)c3ccccc32)CCN1CC(F)F] |
| 64 | 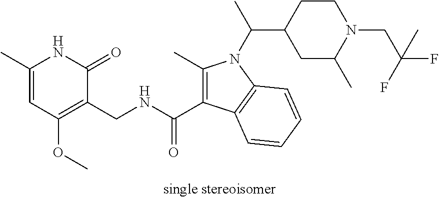 [SMILES: COc1cc(C)[nH]c(=O)c1CNC(=O)c1c(C)n(C(C)C2CCN(CC(C)(F)F)C(C)C2)c2ccccc12] |
| 65 | 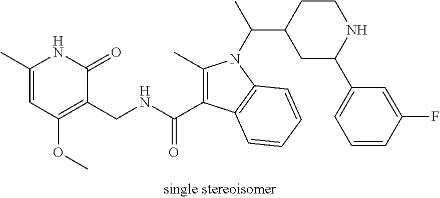 [SMILES: COc1cc(C)[nH]c(=O)c1CNC(=O)c1c(C)n(C(C)C2CCNC(c3cccc(F)c3)C2)c2ccccc12] |
| 66 | 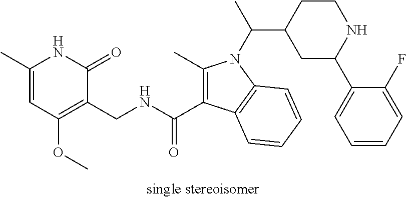 [SMILES: COc1cc(C)[nH]c(=O)c1CNC(=O)c1c(C)n(C(C)C2CCNC(c3ccccc3F)C2)c2ccccc12] |
| 67 | 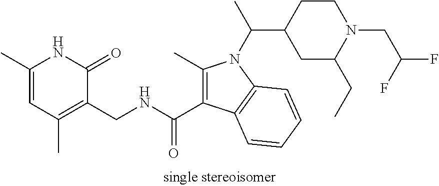 [SMILES: CCC1CC(C(C)n2c(C)c(C(=O)NCc3c(C)cc(C)[nH]c3=O)c3ccccc32)CCN1CC(F)F] |
| 68 | 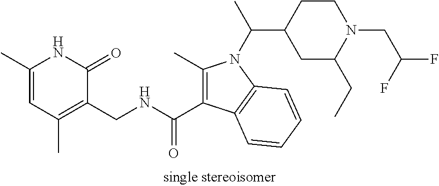 [SMILES: CCC1CC(C(C)n2c(C)c(C(=O)NCc3c(C)cc(C)[nH]c3=O)c3ccccc32)CCN1CC(F)F] |
| 69 | 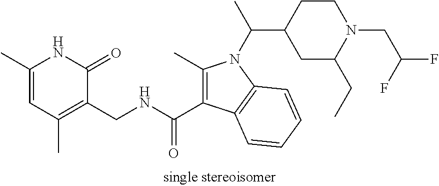 [SMILES: CCC1CC(C(C)n2c(C)c(C(=O)NCc3c(C)cc(C)[nH]c3=O)c3ccccc32)CCN1CC(F)F] |
| 70 | 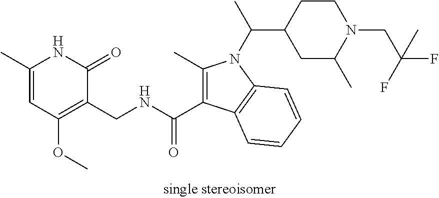 [SMILES: COc1cc(C)[nH]c(=O)c1CNC(=O)c1c(C)n(C(C)C2CCN(CC(C)(F)F)C(C)C2)c2ccccc12] |
| 71 | 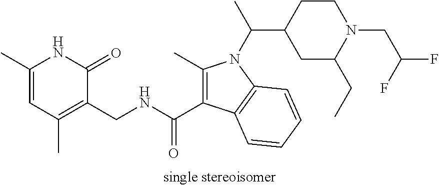 [SMILES: CCC1CC(C(C)n2c(C)c(C(=O)NCc3c(C)cc(C)[nH]c3=O)c3ccccc32)CCN1CC(F)F] |
| 72 | 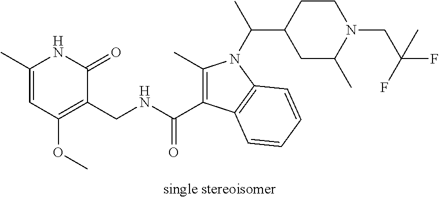 [SMILES: COc1cc(C)[nH]c(=O)c1CNC(=O)c1c(C)n(C(C)C2CCN(CC(C)(F)F)C(C)C2)c2ccccc12] |
| 73 | 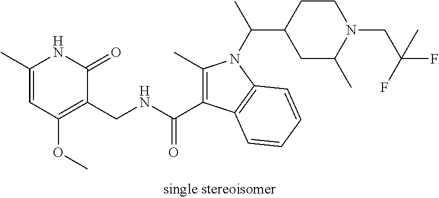 [SMILES: COc1cc(C)[nH]c(=O)c1CNC(=O)c1c(C)n(C(C)C2CCN(CC(C)(F)F)C(C)C2)c2ccccc12] |
| 74 | 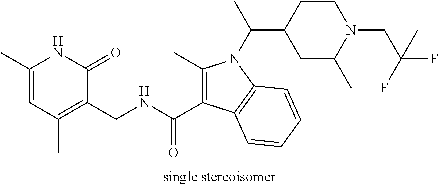 [SMILES: Cc1cc(C)c(CNC(=O)c2c(C)n(C(C)C3CCN(CC(C)(F)F)C(C)C3)c3ccccc23)c(=O)[nH]1] |
| 75 | 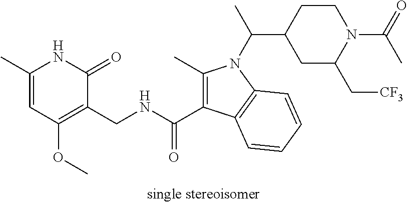 [SMILES: COc1cc(C)[nH]c(=O)c1CNC(=O)c1c(C)n(C(C)C2CCN(C(C)=O)C(CC(F)(F)F)C2)c2ccccc12] |
| 76 | 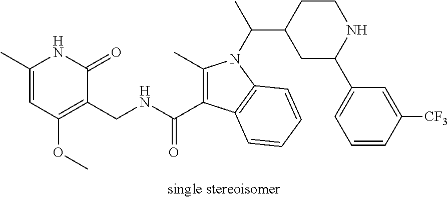 [SMILES: COc1cc(C)[nH]c(=O)c1CNC(=O)c1c(C)n(C(C)C2CCNC(c3cccc(C(F)(F)F)c3)C2)c2ccccc12] |
| 77 | 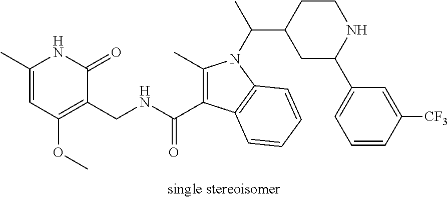 [SMILES: COc1cc(C)[nH]c(=O)c1CNC(=O)c1c(C)n(C(C)C2CCNC(c3cccc(C(F)(F)F)c3)C2)c2ccccc12] |
| 78 | 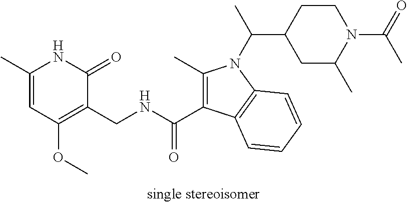 [SMILES: COc1cc(C)[nH]c(=O)c1CNC(=O)c1c(C)n(C(C)C2CCN(C(C)=O)C(C)C2)c2ccccc12] |
| 79 | 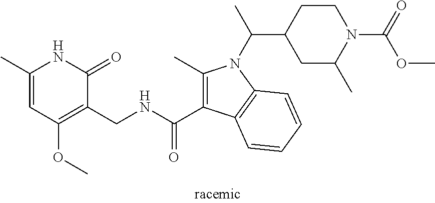 |
| 80 | 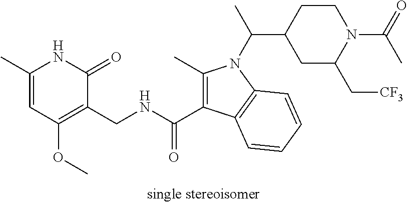 [SMILES: COc1cc(C)[nH]c(=O)c1CNC(=O)c1c(C)n(C(C)C2CCN(C(C)=O)C(CC(F)(F)F)C2)c2ccccc12] |
| 81 | 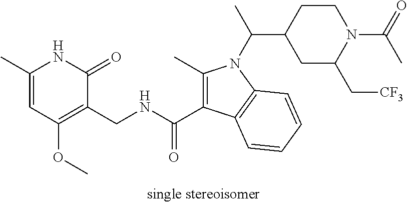 [SMILES: COc1cc(C)[nH]c(=O)c1CNC(=O)c1c(C)n(C(C)C2CCN(C(C)=O)C(CC(F)(F)F)C2)c2ccccc12] |
| 82 | 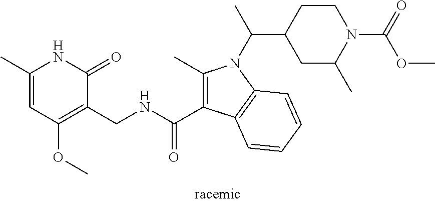 |
| 83 | 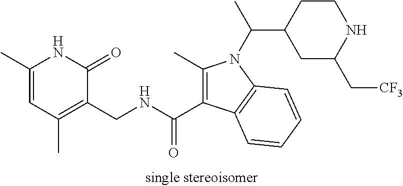 [SMILES: Cc1cc(C)c(CNC(=O)c2c(C)n(C(C)C3CCNC(CC(F)(F)F)C3)c3ccccc23)c(=O)[nH]1] |
| 84 | 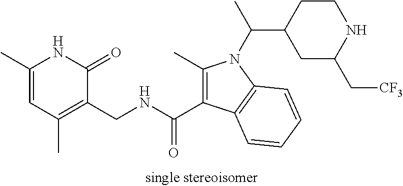 [SMILES: Cc1cc(C)c(CNC(=O)c2c(C)n(C(C)C3CCNC(CC(F)(F)F)C3)c3ccccc23)c(=O)[nH]1] |
| 85 | 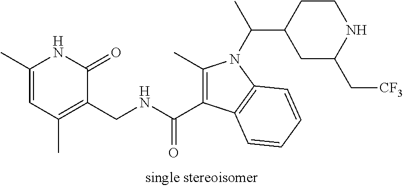 [SMILES: Cc1cc(C)c(CNC(=O)c2c(C)n(C(C)C3CCNC(CC(F)(F)F)C3)c3ccccc23)c(=O)[nH]1] |
| 86 | 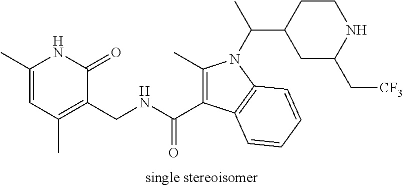 [SMILES: Cc1cc(C)c(CNC(=O)c2c(C)n(C(C)C3CCNC(CC(F)(F)F)C3)c3ccccc23)c(=O)[nH]1] |
| 87 | 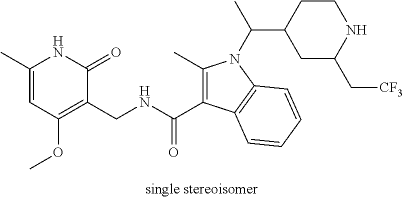 [SMILES: COc1cc(C)[nH]c(=O)c1CNC(=O)c1c(C)n(C(C)C2CCNC(CC(F)(F)F)C2)c2ccccc12] |
| 88 | 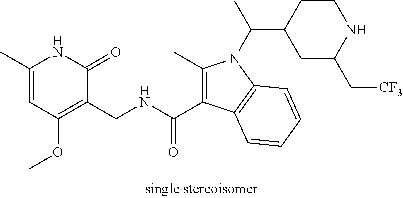 [SMILES: COc1cc(C)[nH]c(=O)c1CNC(=O)c1c(C)n(C(C)C2CCNC(CC(F)(F)F)C2)c2ccccc12] |
| 89 | 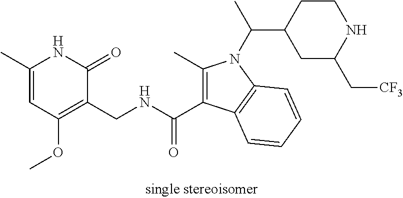 [SMILES: COc1cc(C)[nH]c(=O)c1CNC(=O)c1c(C)n(C(C)C2CCNC(CC(F)(F)F)C2)c2ccccc12] |
| 90 | 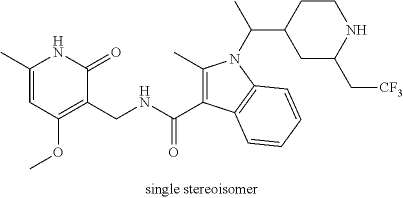 [SMILES: COc1cc(C)[nH]c(=O)c1CNC(=O)c1c(C)n(C(C)C2CCNC(CC(F)(F)F)C2)c2ccccc12] |
| 91 | 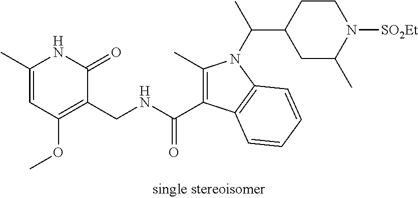 |
| 92 | 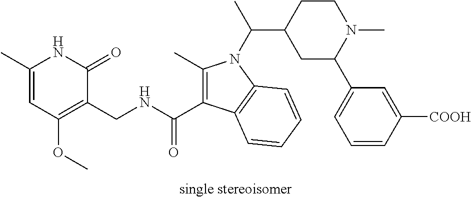 [SMILES: COc1cc(C)[nH]c(=O)c1CNC(=O)c1c(C)n(C(C)C2CCN(C)C(c3cccc(C(=O)O)c3)C2)c2ccccc12] |
| 93 | 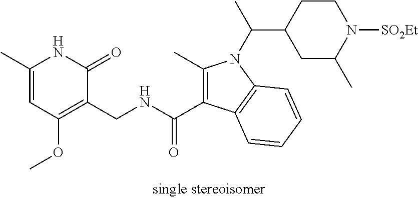 |
| 94 | 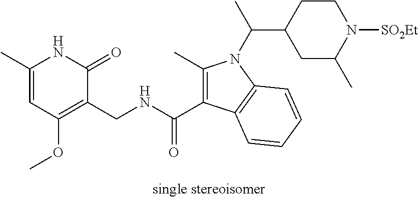 |
| 95 | 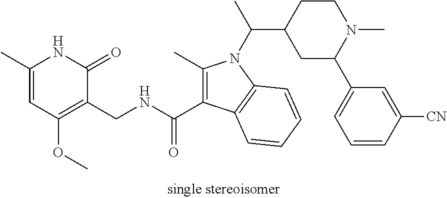 [SMILES: COc1cc(C)[nH]c(=O)c1CNC(=O)c1c(C)n(C(C)C2CCN(C)C(c3cccc(C#N)c3)C2)c2ccccc12] |
| 96 | 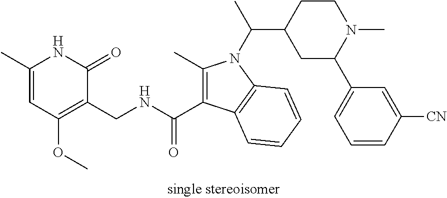 [SMILES: COc1cc(C)[nH]c(=O)c1CNC(=O)c1c(C)n(C(C)C2CCN(C)C(c3cccc(C#N)c3)C2)c2ccccc12] |
| 97 | 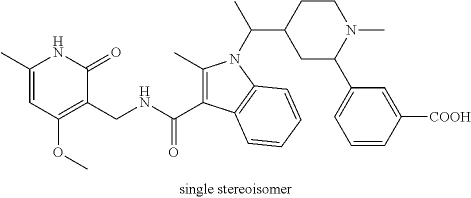 [SMILES: COc1cc(C)[nH]c(=O)c1CNC(=O)c1c(C)n(C(C)C2CCN(C)C(c3cccc(C(=O)O)c3)C2)c2ccccc12] |

### Example 26: EZH2 Assays

IC₅₀ Measurements for Inhibitors Using EZH2

EZH2 Nuc BSA Brij 1.5 (IC50):

Assays were carried out by mixing rPRC2 together with biotinylated oligonucleosome substrates in the presence of the radio-labeled enzyme co-factor, S-adenosyl-L-methionine (³H SAM) (Perkin Elmer) and monitoring the enzymatically mediated transfer of tritiated methyl groups from ³H SAM to histone lysine residues. The amount of resulting tritiated methyl histone product was measured by first capturing the biotinylated oligonucleosomes in streptavidin (SAV) coated FlashPlates (Perkin Elmer), followed by a wash step to remove un-reacted ³H SAM, and then counting on a TopCount NXT 384 well plate scintillation counter (Perkin Elmer). The final assay conditions for EZH2 were as follows: 50 mM Tris Buffer pH 8.5, 1 mM DTT, 69 μM Brij-35 detergent, 5.0 mM MgCl₂, 0.1 mg/mL BSA, 0.2 μM ³H SAM, 0.2 μM biotinylated oligonucleosomes, 3.6 μM H3K27me3 peptide and 2 nM EZH2.

Compound IC₅₀ Measurements were Obtained as Follows:

Compounds were first dissolved in 100% DMSO as 10 mM stock solutions. Ten point dose response curves were generated by dispensing varying amounts of the 10 mM compound solution in 10 wells of the 384 well plate (Echo; Labcyte), pure DMSO was then used to backfill the wells to insure all wells have the same amount of DMSO. A 12.5 μL volume of the HMT enzyme, H3K27me3 peptide and oligonucleosome substrate in assay buffer was added to each well of the assay plate using a Multidrop Combi (ThermoFisher). Compounds were pre-incubated with the enzyme for 20 min, followed by initiation of the methyltransferase reaction by addition of 12.5 μL of ³H SAM in assay buffer (final volume=25 μL). The final concentrations of compounds ranged from a top default concentration of 80 μM down to 0.16 μM in ten 2-fold dilution steps. Reactions were carried out for 60 minutes and quenched with 20 μL per well of 1.96 mM SAH, 50 mM Tris pH 8.5, 200 mM EDTA. Stopped reactions were transferred to SAV coated Flashplates (Perkin Elmer), incubated for 120 min, washed with a plate washer, and then read on the TopCount NXT (1.0 min/well) to measure the amount of methyl histone product formed during the reaction. The amount of methyl histone product was compared with the amount of product formed in the 0% and 100% inhibition control wells allowing the calculation of % Inhibition in the presence of the individual compounds at various concentrations. IC₅₀'s were computed using a 4 parameter fit non-linear curve fitting software package (XLFIT, part of the database package, ActivityBase (IDBS)) where the four parameters were IC₅₀, Hill slope, pre-transitional baseline (0% INH), and post-transitional baseline (100% INH); with the latter two parameters being fixed to zero and 100%, respectively, by default.

EZH2 Pep BSA Brij 4.0 LowE (IC50):

Compound potencies were assessed through incorporation of ³H-SAM into a biotinylated H3 peptide. Specifically, PRC2 containing wt EZH2 (20 pM), (pentameric complexes prepared in-house) was pre-incubated with ³H-SAM (0.9 μM), 2 μM H3K27me3 activating peptide (H₂N-RKQLATKAAR(Kme3)SAPATGGVKKP-amide) and compounds (as 10 point duplicate dose response titrations in DMSO, final assay 0.8% DMSO (v/v)) for 120 min in 50 mM Tris (pH 8.5), 1 mM DTT, 0.07 mM Brij-35, 0.1% BSA, and 0.8% DMSO in a total volume of 12.5 μl. Reaction was initiated with biotinylated H3 substrate peptide (H₂N-RKQLATKAAR(Kme1)SAPATGGVKKP-NTPEGBiot) as a 2 μM stock in 12.5 μl of buffer and allowed to react at room temperature for 5 h. Quenching was accomplished by addition of 20 μl of STOP solution (50 mM Tris (pH 8.5), 200 mM EDTA, 2 mM SAH). 35 μl of the quenched solution was transferred to Streptavidin Flashplates, incubated overnight, washed, and read in a TopCount Reader (Perkin Elmer). IC₅₀s were calculated relative to fully active and no enzyme control wells using non-linear least square four parameter fits.

Table 2 shows the activity of selected compounds of this invention in the EZH2 activity inhibition assays. IC₅₀ values are reported as follows: “A” indicates an IC₅₀ value of less than 100 nM; “B” indicates an IC₅₀ value of 100 nM to 1 μM; “C” indicates an IC₅₀ value of greater than 1 μM and less than 10 μM for each enzyme; “D” indicates an IC₅₀ value of greater than 10 μM for each enzyme; “*(X μM)” indicates that no inhibition was observed at the highest concentration (i.e., X μM) of compound tested; and “ND” is not determined.

TABLE 2
IC₅₀ values for selected compounds.

| | EZH2 | EZH2 pep BSA |
|---|---|---|
| | nuc BSA Brij 1.5 | Brij 4.0 LowE |
| Cmp | (IC50) | (IC50) |
| 1 | A | ND |
| 2 | ND | ND |
| 3 | A | ND |
| 4 | A | ND |
| 5 | ND | A |
| 6 | ND | A |
| 7 | ND | A |
| 8 | A | A |
| 9 | A | ND |
| 10 | A | ND |
| 11 | A | ND |
| 12 | A | ND |
| 13 | A | ND |
| 14 | A | A |
| 15 | A | A |
| 16 | A | ND |
| 17 | A | A |
| 18 | A | A |
| 19 | A | ND |
| 20 | A | ND |
| 21 | A | A |
| 22 | A | ND |
| 23 | B | ND |
| 24 | A | A |
| 25 | ND | A |
| 26 | ND | A |
| 27 | ND | B |
| 28 | ND | A |
| 29 | ND | A |
| 30 | ND | A |
| 31 | A | A |
| 32 | A | ND |
| 33 | A | A |
| 34 | ND | B |
| 35 | ND | A |
| 36 | A | ND |
| 37 | A | ND |
| 38 | A | ND |
| 39 | A | ND |
| 40 | A | A |
| 41 | A | A |
| 42 | A | ND |
| 43 | A | A |
| 44 | ND | A |
| 45 | ND | A |
| 46 | ND | A |
| 47 | ND | B |
| 48 | ND | B |
| 49 | ND | B |
| 50 | ND | A |
| 51 | ND | A |
| 52 | ND | A |
| 53 | ND | A |
| 54 | ND | A |
| 55 | ND | A |
| 56 | ND | A |
| 57 | ND | A |
| 58 | ND | A |
| 59 | ND | A |
| 60 | ND | A |
| 61 | ND | A |
| 62 | ND | A |
| 63 | ND | A |
| 64 | ND | A |
| 65 | ND | A |
| 66 | ND | A |
| 67 | ND | A |
| 68 | ND | A |
| 69 | ND | A |
| 70 | ND | A |
| 71 | ND | A |
| 72 | ND | B |
| 73 | ND | A |
| 74 | ND | A |
| 75 | ND | A |
| 76 | ND | A |
| 77 | ND | A |
| 78 | ND | A |
| 79 | ND | A |
| 80 | ND | A |
| 81 | ND | A |
| 82 | ND | B |
| 83 | ND | A |
| 84 | ND | A |
| 85 | ND | A |
| 86 | ND | A |
| 87 | ND | B |
| 88 | ND | A |
| 89 | ND | A |
| 90 | ND | A |
| 91 | ND | A |
| 92 | ND | A |
| 93 | ND | B |
| 94 | ND | B |
| 95 | ND | A |
| 96 | ND | A |
| 97 | ND | A |

EC₅₀ Measurements for Inhibitors in HeLa Cell Assays

H3K27me3 Alpha Hela Assay (AlphaLISA). Ten different doses of each test compound (in a series of 3-fold dilutions) were plated in duplicate 384-well tissue culture treated plates (Catalog #781080; Greiner Bio One; Monroe, N.C.). Hela cells grown in culture were trypsinized and counted using a Countess® cell counter (Catalog # C10281; Life Technologies, Grand Island, N.Y.). Cell were diluted to 67,000 cells per mL in 10% DMEM (Catalog #10569-010 Life Technologies, Grand Island, N.Y.) and 15 μL (1,000 cells) were plated into each well using the Biotek MicroFlo™ Select Dispenser (BioTek Instruments, Inc. Vermont, USA),) of the 384-well plate. Plates were incubated at 37° C./5% CO₂ for 72 hrs. One of the duplicate plates was processed for HeLa assay and the other for viability.

To the plate processed for AlphaLISA was added 5 μL per well Cell-Histone Lysis buffer (1×) (Catalog # AL009F1 Perkin Elmer; Waltham, Mass.) and the plate was incubated at RT for 30 minutes on a plate shaker with low speed (Model#4625-Q Thermo Scientific; Waltham, Mass.). Then, 10 μL per well Histone Extraction buffer (catalog # AL009F2; Perkin Elmer; Waltham, Mass.) was added and the plate further incubated at RT for 20 min on plate shaker with low speed. To each well was then added 10 μL per well of a 5× mix of anti-K27me3 acceptor beads plus Biotinylated anti-Histone H3 (C-ter) Antibody (diluted to 3 nM final) (Catalog #AL118 Perkin Elmer; Waltham, Mass.). Dilution of the acceptor beads and then anti-Histone H3 was with 1× Histone Detection buffer (Catalog # AL009F3 Perkin Elmer; Waltham, Mass.) which was produced diluted from the 10× stock provided. The plate was sealed with an aluminum plate sealer and incubated at 23° C. for 60 min. We then added 10 μL 5× solution of Streptavidin Donor beads (Catalog #6760002 Perkin Elmer; Waltham, Mass.) (20 μg/mL final in 1× Histone Detection Buffer), sealed the plate with Aluminum plate sealer and incubated at 23° C. for 30 min. The plates were then read using an EnVision-Alpha Reader (model #2104 Perkin Elmer; Waltham, Mass.).

Cell viability was assayed by adding 15 μL of Cell Titer Glo ((Catalog #G7571 Promega Madison, Wis.) to each well with cells with media. The plates were incubated foat RT for 15-20 minutes on a plate shaker at low speed. The plates were then read using an EnVision-Alpha Reader (model #2104 Perkin Elmer; Waltham, Mass.).

Data from both assays was analyzed using Assay Assistant (Constellation Pharmaceuticals In-house product) and Activity Base (IDBS Ltd, Surrey, UK) template. Data files were imported to Assay Assistant and assay conditions were specified. A unique Analysis ID was created and the data files exported to Activity Base. An analysis template was created on Activity Base to measure dose-dependent inhibition of H3K27me3 mark and cell viability respectively. Readout of DMSO wells were used to normalize the data. Resulting curves were fitted using Activity base software Model 205 (IDBS Ltd, Surrey, UK). The data was checked for quality, validated and integrated in excel format using SARview (IDBS Ltd, Surrey, UK).

Table 3 shows the activity of selected compounds of this invention in the HeLa cell assays described above. EC₅₀ values are reported as follows: “A” indicates an EC₅₀ value of less than 400 nM; “B” indicates an EC₅₀ value of 400 nM to 2 μM; “C” indicates an EC₅₀ value of greater than 2 μM; and “ND” is not determined.

TABLE 3
EC₅₀ values for selected compounds.

| | EZH2 Cell |
|---|---|
| | H3K27me3 |
| | Alpha HeLa |
| Cmp | (EC50 K27me3) |
| 1 | C |
| 4 | C |
| 5 | A |
| 6 | A |
| 7 | A |
| 8 | C |
| 9 | C |
| 10 | A |
| 11 | C |
| 13 | B |
| 14 | A |
| 15 | A |
| 17 | A |
| 18 | A |
| 19 | C |
| 20 | B |
| 21 | A |
| 22 | B |
| 23 | C |
| 24 | A |
| 25 | C |
| 26 | A |
| 27 | C |
| 28 | C |
| 29 | C |
| 30 | C |
| 31 | B |
| 32 | C |
| 33 | B |
| 34 | C |
| 35 | A |
| 36 | C |
| 37 | B |
| 38 | C |
| 39 | C |
| 40 | A |
| 41 | A |
| 42 | B |
| 43 | A |
| 44 | C |
| 45 | A |
| 46 | A |
| 47 | C |
| 66 | B |
| 68 | C |
| 69 | C |
| 70 | B |
| 71 | A |
| 72 | C |
| 73 | A |
| 84 | B |
| 88 | C |
| 89 | B |
| 90 | B |
| 91 | C |
| 92 | B |
| 95 | B |
| 96 | B |
| 97 | C |

### Example 26: In Vitro Metabolism

The in vitro metabolism of compound 21 was evaluated by disappearance of compound in mouse, rat, dog and human liver microsomes at 10 μM compound concentration. The amount of compound 21 remaining after incubation with cryopreserved mouse, rat, dog and human liver microsomes (0.7 mg protein/mL) was determined by LC/MS/MS at different time points (t (min)=0, 5, 10, 20, 30, 60) and fit to the equations for first order kinetics (below) to afford the intrinsic clearance (μL/min/mg protein).

Cₜ=C₀*e^{-kt}

Cₜ=(½)*C₀

t_{1/2}=ln 2/k=

0.693/k

CL=V_{d}*k

Vd=1.429 mL/mg

Table 4 shows the activity of compound 21 in liver microsomes. Data is expressed in μL/min/mg protein.

TABLE 4

| | |
|---|---|
| Mouse | 55 |
| Rat | 35 |
| Dog | 35 |
| Human | 39 |

### Example 27: Plasma-Tissue Distribution

1 h after dosing a group of mice (3 mice/group) via oral gavage at 100 mg/kg of compound 21, blood is collected from the submandibular or saphenous vein (˜30 μL) and tissue is collected from the biceps femoris (both sides). After tissue preparation and homogenization, plasma and tissue samples are assayed for parent drug using LC/MS/MS methodologies with internal standard. The glucuronide metabolites (parent mass +176) are also monitored via LC/MS/MS and compared to the internal standard for quantitation.

Table 5 shows the tissue distribution and glucoronide formation with compound 21 recorded 1 hour after dosing in healthy male BALB/C mice.

TABLE 5

| | | |
|---|---|---|
| Distribution Study | Conc. in Plasma (ng/mL) | 8373 |
| | Conc. in Muscle (ng/g) | 7240 |
| | Ratio Muscle/Plasma | 0.86 |
| Glucoronide-P | Peak Area Ratio in Plasma | 5.09 |
| | Peak Area Ratio in Muscle | 0.80 |

Table 6 shows the mean plasma and mean tumor concentration from a 7-day study of healthy female severe combined immunodeficiency (SCID) mice with treatment of compound 21.

TABLE 6

| Dose | Mean Plasma | Mean Tumor |
|---|---|---|
| (P.O. BID) | Conc (ng/mL) | Conc (ng/g) |
| 100 mg/kg | 280 | 451 |
| 200 mg/kg | 1597 | 693 |

1. A compound of the formula (I): 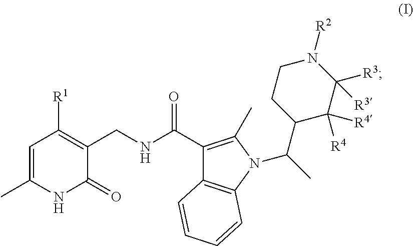
or a pharmaceutically acceptable salt thereof, wherein
R¹ is (C₁-C₄)alkyl, (C₁-C₃)alkoxy, or OCHF₂;
R² is hydrogen, methyl, ethyl, thiazolyl, —SO₂(C₁-C₃)alkyl, —COCH₃, —COOCH₃, —C(CH₃)₂NH(C₁-C₃)alkyl, —C(CH₃)₂COOH, —CH₂R⁵, —C(O)OR⁶, —C(O)R⁷, —CHR⁸R⁹, —CH₂(CO)R¹⁰, isoxazolyl substituted with (C₁-C₃)alkyl, or oxetanyl optionally substituted with hydroxycarbonyl(C₁-C₃)alkyl or hydroxy(C₁-C₃)alkyl;
R³, R³′, R⁴ and R^{4′} are each independently hydrogen, CF₃, CH₃, CH₂CH₃, CH₂F, CHF₂, CH₂CF₃, ═O, CH₂OH, or phenyl optionally substituted with (C₁-C₃)alkyl, halo(C₁-C₃)alkyl, halo, cyano, or COOH, provided that at least one of R³, R³′, R⁴ and R^{4′} is not hydrogen; or
R³ and R⁴ or R^{3′} and R^{4′} are taken together form a phenyl or cyclohexyl ring;
R⁵ is cyclopropyl, CHF₂, —C(CH₃)₂COOCH₃, —C(CH₃)₂COOH, —CF₂CH₂pyrrolidinyl, -phenyl(CO)phenyl, —CH₂OCF₃, —C(CH₃)₂CH₂NH₂, CF₃, —CH₂NHCH₂CF₃, pyridinyl substituted with (C₂-C₄)alkyl, or isoxazolyl substituted with one or more (C₁-C₃)alkyl;
R⁶ is di(C₁-C₃)alkylamino(C₁-C₃)alkyl, pyrrolidinyl substituted with (C₁-C₃)alkyl, cyclopropyl substituted with (C₁-C₃)alkyl, azetidinyl substituted with (C₁-C₃)alkyl, or piperidinyl substituted with (C₁-C₃)alkyl;
R⁷ is —CH₂-cyclopentyl, —CH₂-cyclopropyl, —CH₂-tetrahydropyranyl, —CH₂NHCH₂CF₃, pyridazinyl, —CH₂CF₃, piperidinyl substituted with (C₁-C₃)alkoxy, —CH₂-azetidinyl substituted with one or more fluoro, pyrazinyl substituted with (C₁-C₃)alkyl, imidazolyl substituted with (C₁-C₃)alkyl, pyridinyl substituted with (C₁-C₃)alkyl, or pyrrolidinyl substituted with (C₁-C₃)alkoxy or hydroxyl;
R⁸ is CF₃, CH₃, or —NOCH₃;
R⁹ is pyridinyl optionally substituted with one or two (C₁-C₃)alkyl, phenyl substituted with cyano or one or more halo, or pyrazolyl substituted with one or two groups selected from (C₁-C₃)alkyl and halo; and
R¹⁰ is NH(C₁-C₃)alkyl, (C₂-C₄)alkyl, cyclopenyl, cyclobutyl optionally substituted with CF₃, or cyclopropyl optionally substituted with (C₁-C₃)alkyl, halo, or CF₃.

2. The compound of claim 1, wherein the compound is of the formula (II): 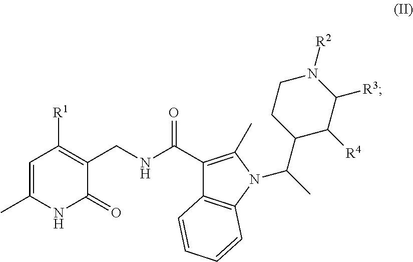
or a pharmaceutically acceptable salt thereof.

3. The compound of claim 1 or 2, wherein the compound is of the Formula (III): 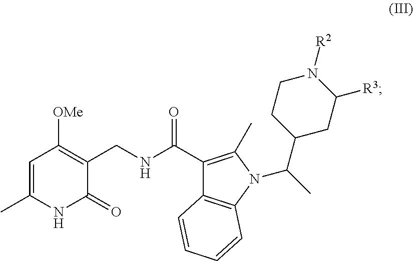
or a pharmaceutically acceptable salt of either of the foregoing.

4. The compound of claim 3, wherein R³ is CF₃, CH₂CH₃, CH₃, or halophenyl.

5. The compound of claim 4, wherein R² is hydrogen, methyl, ethyl, thiazolyl, —C(CH₃)₂NH(C₁-C₃)alkyl, —C(CH₃)₂COOH, —CH₂R⁵, —C(O)OR⁶, —C(O)R⁷, —CHR⁸R⁹, —CH₂(CO)R¹⁰, or isoxazolyl substituted with (C₁-C₃)alkyl.

6. The compound of claim 5, wherein R² is hydrogen, methyl, ethyl, —CH₂R⁵, —C(O)OR⁶, —C(O)R⁷, —CHR⁸R⁹, or —CH₂(CO)R¹⁰.

7. The compound of claim 6, wherein R² is hydrogen, methyl, ethyl, —CH₂R⁵, or —C(O)R⁷.

8. The compound of claim 7, wherein R⁵ is cyclopropyl, CHF₂, -phenyl(CO)phenyl, —CH₂OCF₃, CF₃, pyridinyl substituted with (C₂-C₄)alkyl, or isoxazolyl substituted with one or more (C₁-C₃)alkyl.

9. The compound of claim 8, wherein R⁵ is CHF₂, CF₃, or pyridinyl substituted with (C₂-C₄)alkyl.

10. The compound of claim 9, wherein R⁵ is CHF₂ or CF₃.

11. The compound of claim 10, wherein R⁶ is cyclopropyl substituted with (C₁-C₃)alkyl or piperidinyl substituted with (C₁-C₃)alkyl.

12. The compound of claim 11, wherein R⁷ is —CH₂-cyclopentyl, —CH₂-cyclopropyl, —CH₂NHCH₂CF₃, —CH₂CF₃, pyrazinyl substituted with (C₁-C₃)alkyl, imidazolyl substituted with (C₁-C₃)alkyl, or pyrrolidinyl substituted with (C₁-C₃)alkoxy.

13. The compound of claim 12, wherein R⁸ is CF₃ or CH₃.

14. The compound of claim 13, wherein R⁹ is pyridinyl optionally substituted with one or two (C₁-C₃)alkyl; or phenyl substituted with cyano or one or more halo.

15. The compound of claim 14, wherein R¹⁰ is NH(C₁-C₃)alkyl, (C₂-C₄)alkyl, cyclopenyl, cyclobutyl optionally substituted with CF₃, or cyclopropyl optionally substituted with (C₁-C₃)alkyl, halo, or CF₃.

16. The compound of claim 1, wherein the compound is of Formula (IV): 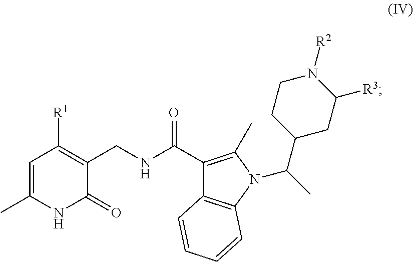
or a pharmaceutically acceptable salt thereof, wherein
R¹ is (C₁-C₄)alkyl or (C₁-C₃)alkoxy;
R² is hydrogen, methyl, ethyl, oxetanyl, —CH₂cyclopropyl, —CH₂CHF₂, CH₂CF₃, —COCH₃, or —SO₂Et; and
R³ is methyl, ethyl, CF₃, ═O, —CH₂CH₂F, —CH₂OH, —CHF₂, —CH₂CF₃, halophenyl, or trifluoromethylphenyl.

17. The compound of claim 16, wherein R¹ is methyl or methoxy.

18. The compound of claim 17, wherein R² is hydrogen, methyl, or ethyl; and R³ is CF₃ or halophenyl.

19. (canceled)

20. The compound of claim 1, wherein R³ is CF₃.

21. The compound of claim 1, wherein the compound is selected from 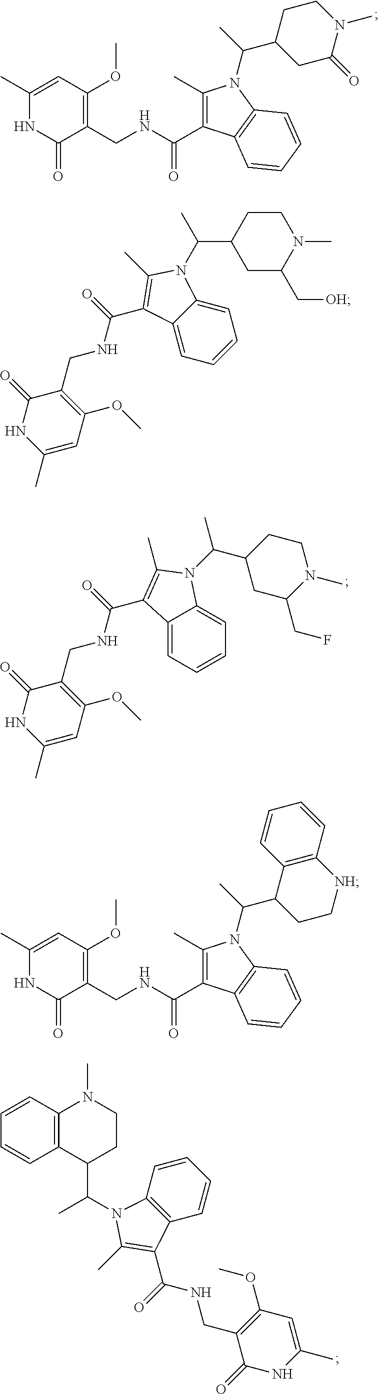 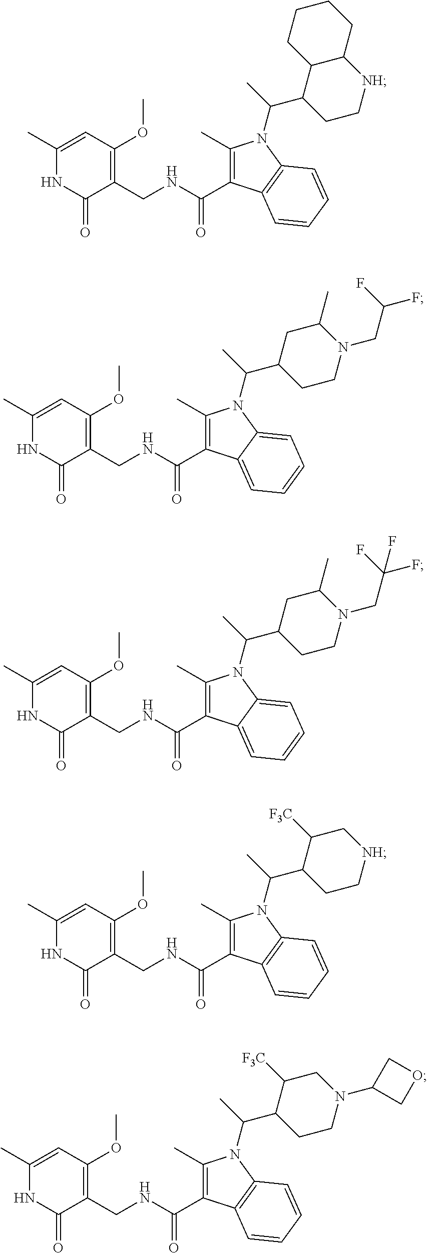 [SMILES: COc1cc(C)[nH]c(=O)c1CNC(=O)c1c(C)n(C(C)C2CCNC3CCCCC32)c2ccccc12 ; COc1cc(C)[nH]c(=O)c1CNC(=O)c1c(C)n(C(C)C2CCN(CC(F)F)C(C)C2)c2ccccc12 ; COc1cc(C)[nH]c(=O)c1CNC(=O)c1c(C)n(C(C)C2CCN(CC(F)(F)F)C(C)C2)c2ccccc12 ; COc1cc(C)[nH]c(=O)c1CNC(=O)c1c(C)n(C(C)C2CCNCC2C(F)(F)F)c2ccccc12 ; COc1cc(C)[nH]c(=O)c1CNC(=O)c1c(C)n(C(C)C2CCN(C3COC3)CC2C(F)(F)F)c2ccccc12] 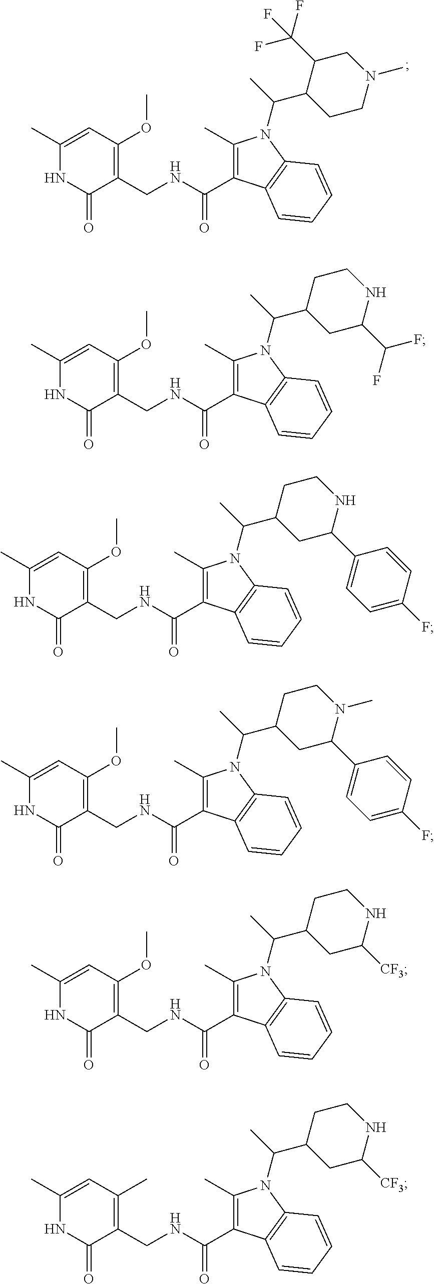 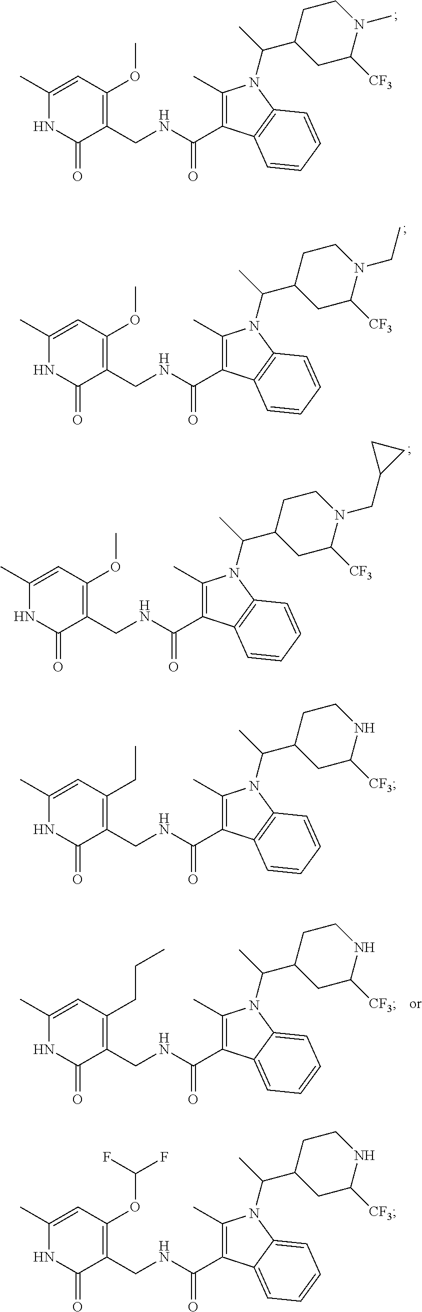
or a pharmaceutically acceptable salt thereof.

22. The compound of claim 1, wherein the compound is selected from 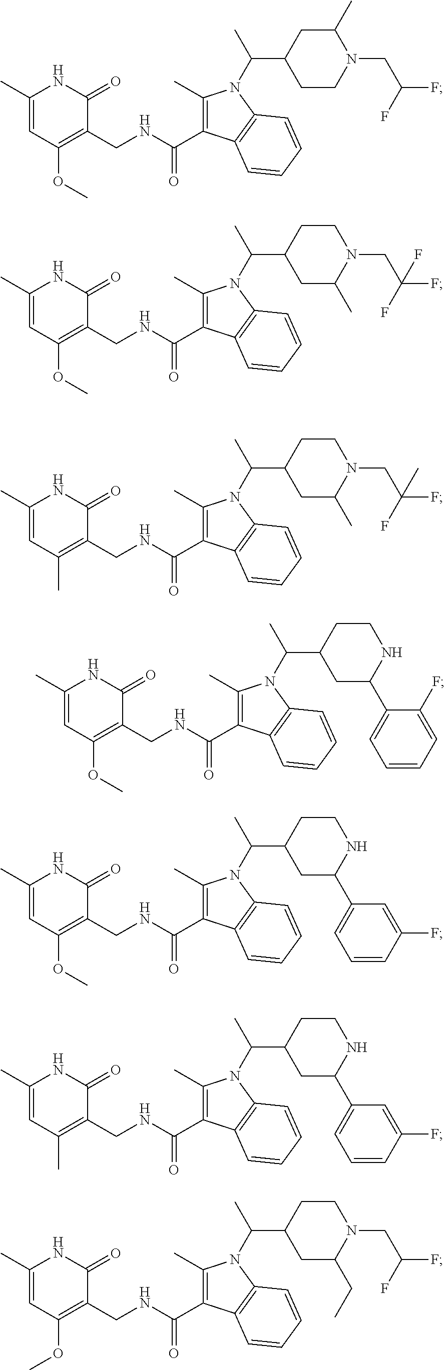 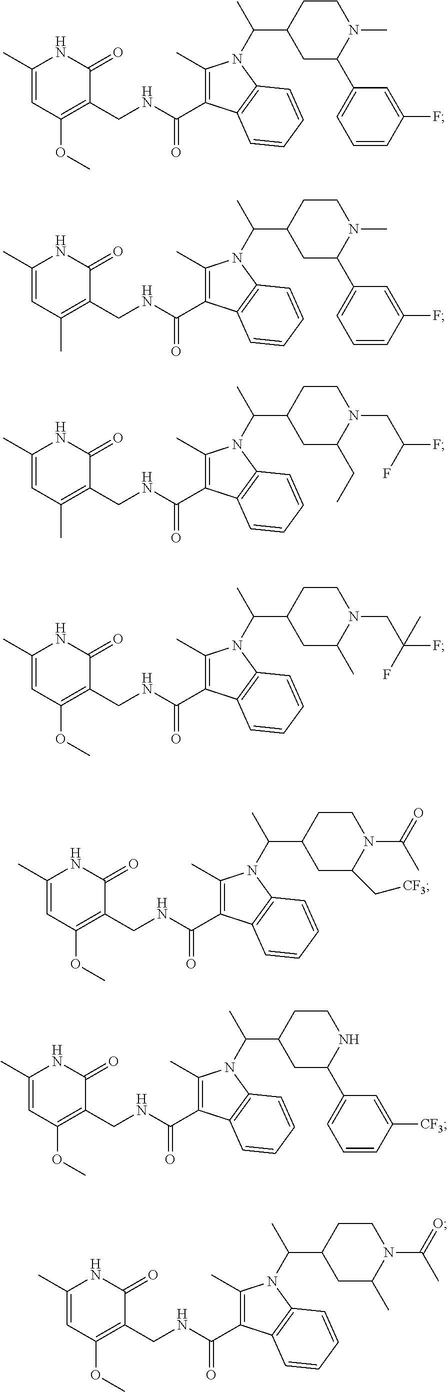 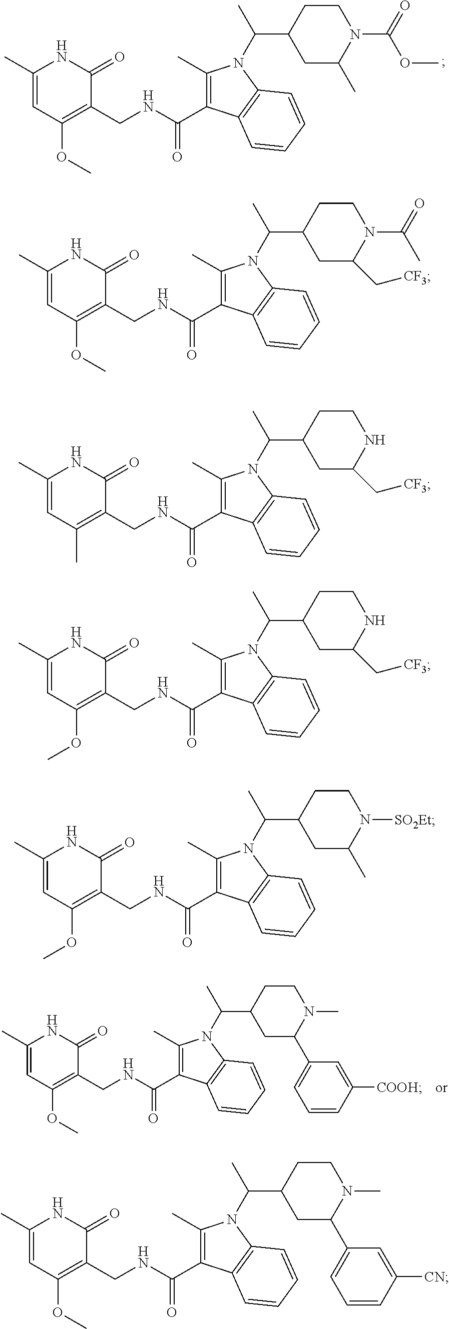
or a pharmaceutically acceptable salt thereof.

23. A composition comprising a compound of claim 1, or a pharmaceutically acceptable salt thereof; and a pharmaceutically acceptable carrier.

24. A method of treating cancer in a patient comprising the step of administering to the patient in need thereof a compound according to claim 1, or a pharmaceutically acceptable salt thereof.

25-31. (canceled)
